(19) United States
(12) Patent Application Publication
(10) Pub. No.: US 2022/0177550 A1
(43) Pub. Date: Jun. 9, 2022

(54) CHIMERIC PROTEINS AND CHIMERIC PROTEIN COMPLEXES DIRECTED TO FMS-LIKE TYROSINE KINASE 3 (FLT3)

(71) Applicants: Orionis Biosciences, Inc., Waltham, MA (US); Orionis Biosciences BV, Zwijnaarde (BE)

(72) Inventors: Nikolai KLEY, Waltham, MA (US); Erik DEPLA, Zwijnaarde (BE); Lennart ZABEAU, Zwijnaarde (BE); Jan TAVERNIER, Zwijnaarde (BE)

(21) Appl. No.: 17/598,570

(22) PCT Filed: Mar. 27, 2020
(86) PCT No.: PCT/US20/25421

§ 371 Date: Sep. 27, 2021

Related U.S. Application Data
(60) Provisional application No. 62/825,580, filed on Mar. 28, 2019.

(51) Int. Cl. C07K 16/00; A61P 37/04; C07K 14/545; C07K 14/56; C12N 9/12

(52) U.S. Cl. CPC C07K 16/00; A61P 37/04; C07K 14/545; A61K 38/00; C12N 9/12; C12Y 207/10001; C07K 14/56

(57) ABSTRACT

Chimeric proteins or chimeric protein complexes, such as an Fc-based chimeric protein complexes directed to FMS-like tyrosine kinase 3 (FLT3), optionally composed of a FMS-like tyrosine kinase 3L (FLT3L) domain and human cytokines, which find use in, e.g., cancer treatments, are described.

### CROSS-REFERENCE TO RELATED APPLICATIONS

This application claims the benefit of and priority to U.S. Provisional Patent Application No. 62/825,580 filed Mar. 28, 2019, the content of which is hereby incorporated by reference in its entirety.

### FIELD

FMS-like tyrosine kinase 3 ligand (FLT3L) or portions thereof linked to or complexed with a signaling agent, for instance, without limitation, human IFNα2, IFNα1, IFNβ, and IL-1β, which finds use in, e.g. cancer treatments, is described.

### SEQUENCE LISTING

The instant application contains a Sequence Listing that has been submitted in ASCII format via EFS-Web and is hereby incorporated by reference in its entirety. Said ASCII copy, created on Mar. 27, 2020, is named ORN-062PC_B_ST25.txt and is 159,744 bytes in size.

### BACKGROUND

FMS-like tyrosine kinase 3 (FLT3) is expressed on the surface of hematopoietic progenitor cells and some differentiated immune cells. Signaling of FLT3 is important for the normal development of hematopoietic stem cells and progenitor cells. The FLT3 gene is one of the most frequently mutated genes in acute myeloid leukemia (AML). Further, FMS-like tyrosine kinase 3 ligand (FLT3L) agents find use in priming and stimulating the immune system, e.g., altering the number of dendritic cells. FLT3 is thus a marker for certain immune cells as well as a functionally important molecule that regulates immune cells and mediates immune-stimulatory functions of its ligand, FLT3L.

Cytokines are naturally occurring substances capable of modulating cellular growth and differentiation. Cytokines play important roles in a variety of physiological processes including, for example, metabolism, respiration, sleep, excretion, healing, movement, reproduction, mood, stress, tissue function, immune function, sensory perception, and growth and development.

Clinically, cytokines would seem to be applicable to the treatment of a variety of diseases and disorders including, for example, cancers. However, the administration of these soluble agents is not without risks. The therapeutic use of cytokines is often associated with systemic toxicity and deleterious side effects, thus limiting the dose levels that these agents can be used at in various treatment regimens. A possible solution to these problems with cytokine agents, is a chimeric protein or chimeric protein complex comprising a signaling agent (e.g., cytokine), connected to or in complex with a targeting element, in which the signaling agent is wild type or modified (e.g. by mutation) to cause an attenuation of the signaling agent's activity (e.g., substantially reducing its ability to interact with/engage its receptor) in a manner such that its effector function can be induced, restored and/or recovered upon binding of the targeting element to its target (e.g., antigen on target cell).

Combining in an agent several properties, including FLT3-targeting, including with FLT3L and its intrinsic effector function at its cognate receptor FLT3, and additional effector functions, such as cytokine effector functions, including inducible cytokine effector functions, would be of considerable interest in the creation of novel, multifunctional protein biologics that would function as immune system modulators for the treatment of cancer and other diseases. However, such chimeric proteins or chimeric protein complexes incorporating FLT3-targeting moieties, such as FLT3L, and additional other signaling effectors, including cytokines and cytokines such as interferons, interleukins and tumor necrosis factor family members, are amenable to therapeutic use only if certain conditions are met. These include, for example:
a) important functional characteristics of such agents, including retention of FLT3-binding in conjunction with delivery of a functional signaling agent signal, e.g., cytokine signal, including an inducible cytokine signal at a FLT3-positive target cell to avoid cytokine cross-reactivities towards undesired target cells, and associated systemic side effects and toxicities and
b) important biochemical, biophysical, pharmacological and pharmaceutical characteristics, such as the ability to be produced in sufficiently homogenous form (ideally single product species) and at a large scale, an in vivo half-life that ensures adequate time of exposure to the drug to elicit a therapeutically beneficial effect, a proper size to avoid rapid clearance or limited tissue penetrance and bio-distribution, and other properties that ensure adequate solubility, stability and storage without significant loss of function.

Importantly, all or substantially most of the above properties should be achieved without a loss of the targeting selectivity and delivery of intrinsic or conditional effector functions to desired targets, including induction and/or restoration of conditional effector function for activity-attenuated cytokines at a therapeutic target(s).

There is a need in the art where such desirable properties of the biologic can be achieved while maintaining the tolerability and therapeutic index of the biologic. Further, there is a need for effector function-encoding biologics that are amenable to production for use as a therapy to the treatment or prevention of disease.

### SUMMARY OF THE INVENTION

Accordingly, in some aspects, the present invention relates to chimeric protein or chimeric protein complex, such as an Fc-based chimeric protein complex comprising one or more targeting moieties which specifically bind to an antigen or receptor of interest, such as FMS-like tyrosine kinase 3 (FLT3). In various embodiments, the targeting moiety functionally modulates the antigen or receptor of interest. In some embodiments, the targeting moiety binds but does not functionally modulate the antigen or receptor of interest. In some embodiments, the targeting moiety comprises FLT3L or the extracellular domain of FLT3L, or respective portions thereof.

In some embodiments the FLT3L-ECD, or variants thereof, is present in two copies on the same polypeptide (a single chain dimeric FLT3L construct). In some embodiments the FLT3L-ECD, or variants thereof, is present in a single copy on each of two different polypeptides that can otherwise dimerize, such as in a Fc-based chimeric protein complex. In some embodiments the FLT3L is a FLT3L-ECD, or a portion or variant thereof, that comprises a mutation that reduces intermolecular FLT3L-ECD homodimerization (i.e., dimerization of FLT3L domains on separate FLT3L-ECD containing molecules that would not otherwise readily dimerize by other mechanisms), and/or favoring intramolecular FLT3L-ECD dimerization (i.e., dimerization of two copies of FLT3L-ECDs contained within the same, single polypeptide, i.e., a single chain dimeric FLT3L construct), or dimerization of single FLT3-ECDs, or variants thereof, on two different polypeptides that can otherwise dimerize within a chimeric protein complex, such as an Fc-based chimeric protein complex. In some embodiments, the mutation in the FLT3L-ECD, or variant thereof, is L27D (with reference to any one of SEQ ID NOs: 2-4 or L24D with regard to SEQ ID NO: 5). In some embodiments, mutation L27D (with reference to any one of SEQ ID NOs: 2-4 or L24D with regard to SEQ ID NO: 5), or functionally similar mutations in the FLT3-ECD, or variants thereof, favor the formation of more homogenous forms of chimeric proteins and chimeric protein complexes, avoiding formation of higher molecular weight aggregates that would be substantially detrimental to scale up production of the FLT3-targeted construct and in vivo safety of such constructs (e.g., risk of immunoreactivity, loss of activity etc.).

In embodiments, the invention provides a FLT3L domain which is a single chain dimer of the formula A-B-C, wherein:
A is an amino acid sequence having at least 90% identity, or having at least 95% identity, or having at least 97% identity, or having at least 98% identity, or having at least 99% identity with any one of SEQ ID NOs: 2-5,
B is a flexible linker which is substantially comprised of glycine and serine residues, optionally wherein the flexible linker comprises (Gly₄Ser)ₙ, where n is from about 1 to about 8, optionally wherein the flexible linker comprises one or more of SEQ ID NO: 10-SEQ ID NO: 17, and
C is an amino acid sequence having at least 90% identity, or having at least 95% identity, or having at least 97% identity, or having at least 98% identity, or having at least 99% identity with any one of SEQ ID NOs: 2-5.

The chimeric protein or chimeric protein complex, such as an Fc-based chimeric protein complex in accordance with embodiments of the present invention also comprises a signaling agent or a modified form thereof, the signaling agent as described herein, for instance, without limitation, human IFNα2, IFNα1, IFNβ, and IL-1β. In embodiments, the chimeric protein or chimeric protein complex, such as an Fc-based chimeric protein complex also comprises one or more linkers.

In some embodiments, the signaling agent can be a wild type signaling agent as described herein, for instance, without limitation, human IFNα2, IFNα1, IFNβ, and IL-1β. In some embodiments, incorporation of a wild type signaling agent into a chimeric protein and/or chimeric protein complex causes attenuation of its activity “attenuation by fusion”). In other embodiments, the signaling agent can be modified to comprise one or more mutations. In some embodiments, mutations in the signaling agent attenuate is activity compared to wild type signaling agent (“attenuation by mutation”). In some embodiments, mutations in the signaling agent may improve its pharmaceutical properties compared to wild type signaling agent. The one or more mutations introduced into the signaling agent can confer various improved properties upon the chimeric proteins and chimeric protein complexes compared to chimeric proteins and chimeric protein complexes with an unmodified (e.g., wild type) signaling agent, including a Fc-based chimeric protein complex compared to an Fc-based chimeric protein complex with an unmodified (e.g., wild type) signaling agent. For example, the signaling agent can be a mutant human signaling agent as described herein, for instance, without limitation, human IFNα2, IFNα1, IFNβ, and IL-1β, having one or more mutations that confer improved safety and/or pharmaceutical properties as compared to the wild type signaling agent as described herein, for instance, without limitation, human IFNα2, IFNα1, IFNβ, and IL-1β. In various embodiments, the one or more mutations can confer improved safety as compared to a wild type signaling agent, reduced affinity for the signaling agent's receptor, or reduced bioactivity for the signaling agent's receptor. In some embodiments, the one or more mutations allow for attenuation of the signaling agent's activity; for example, agonistic or antagonistic activity of the signaling agent may be attenuated. In some embodiments, one or more mutations of the modified signaling agent convert the signaling agent's activity from agonistic to antagonistic. In various embodiments, the mutation(s) confer reduced affinity or activity that is restorable by attachment to one or more targeting moiety or upon inclusion in a chimeric protein complex, such as an Fc-based chimeric protein complex, as disclosed herein. Further, in various embodiments, the mutation(s) confer substantially reduced or ablated affinity or activity that is not substantially restorable by attachment to a targeting moiety or upon inclusion in an Fc-based chimeric protein complex as disclosed herein.

In various embodiments, a targeting moiety is directed against an immune cell or a tumor cell or a component of the disease microenvironment, such that it directly or indirectly recruits immune cells to tumor cells or to the tumor microenvironment, or other disease microenvironment. Non-limiting examples of an immune cell include a dendritic cell, a T cell, a B cell, a macrophage, a neutrophil, mast cell, myeloid derived suppressor cell, or a NK cell. In some embodiments, a targeting moiety is directed to a hematopoietic stem cell (HSC), early progenitor cell, immature thymocyte, or steady state dendritic cell (DC). In embodiments, the targeting is to a dendritic cell, such as a conventional dendritic cell (cDC) or plasmacytoid dendritic cells (pDC). In embodiments, the targeting is to a cDC, optionally being cDC-1, migratory DCs, cDC-2, and Flt3+ DCs. In some embodiments, the targeting moiety may increase a number of dendritic cells. In some embodiments, a targeting moiety of the present chimeric protein and chimeric protein complex, including a Fc-based chimeric protein complex, enhances tumor antigen presentation, optionally by dendritic cells. In some embodiments a targeting moiety directs distribution of a chimeric protein or chimeric protein complex to a disease tissue or disease microenvironment.

In some embodiments, the chimeric protein and chimeric protein complex comprises a single chain dimeric FLT3 ligand. In some embodiments the chimeric protein and chimeric protein complex comprises a single chain dimeric FLT3L that consists of a tandem repeat of a FLT3L monomer (i.e., a single chain dimeric FLT3L), or a portion or variant thereof, such as the FLT3L-ECD (extracellular domain), and in which the individual monomers (e.g., FLT3L-ECDs) are connected by a linker. In some embodiments, the chimeric protein and chimeric protein complex comprises a single chain dimeric FLT3L-ECD, or portion or variant thereof, in which the two FLT3L-ECD monomers, connected via a linker, are further connect to one or more polypeptides via one or more linkers, such polypeptides comprising a signaling agent or a scaffold protein or a scaffold protein and a signaling agent, or, in each case, a modified signaling agent. In some embodiments a targeting moiety in a chimeric protein or chimeric protein complex that incorporates a single chain dimeric FLT3L-ECD, or portion or variant thereof, directs distribution of the chimeric protein or chimeric protein complex to a disease tissue or disease microenvironment.

In some embodiments, a targeting moiety targets a cellular antigen. In some embodiments a targeting moiety targets a non-cellular structure.

In some embodiments, a targeting moiety comprises the extracellular domain (ECD) of FLT3L, or portion or variant thereof, such as an ECD of FLT3L that comprises a mutation that reduces intermolecular FLT3L-ECD homodimerization (i.e., dimerization of FLT3L ECD domains present on separate FLT3L-ECD containing molecules and that would not readily dimerize otherwise by other mechanisms), and favoring intramolecular FLT3L-ECD dimerization (i.e., dimerization of two copies of FLT3L-ECDs contained within the same, single polypeptide, i.e., a single chain dimeric FLT3L construct), or dimerization of single FLT3-ECDs on separate polypeptides that can otherwise dimerize within a specific chimeric protein complex, such as Fc chains in an Fc-based chimeric protein complex. In some embodiments, the mutation in the FLT3L-ECD is L27D (with reference to any one of SEQ ID NOs: 2-4 or L24D with regard to SEQ ID NO: 5)). In some embodiments the mutation(s) with similar functional effect on FLT3L-ECD homodimerization, is in residues 25-30 and/or 63-68 of FLT3L (with reference to SEQ ID NO: 2).

In various embodiments, the present chimeric proteins or chimeric protein complexes, such as an Fc-based chimeric protein complexes find use in a patient having various diseases or disorders such as one or more of cancer, infections, immune disorders, autoimmune and/or neurodegenerative disease, cardiovascular diseases, wound, ischemia-related diseases, metabolic diseases, and/or many other diseases and disorders. The present invention encompasses various methods of treating or preventing diseases and disorders, for instance, various type of cancer and an autoimmune and/or neurodegenerative disease. In some embodiments, the cancer is acute myeloid leukemia (AML).

### BRIEF DESCRIPTION OF DRAWINGS

FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, 19A-F, 21A-F, 22A-F, 24A-H, and 25A-L show various non-limiting illustrative schematics of the Fc-based chimeric protein complexes of the present invention. In embodiments, each schematic is a composition of the present invention. Where applicable in the figures, “TM” refers to a “targeting moiety” as described herein, “SA” refers to a “signaling agent” as described herein, [symbol] is an optional “linker” as described herein, the two long parallel rectangles are human Fc domains, e.g. from IgG1, from IgG2, or from IgG4, as described herein and optionally with effector knock-out and/or stabilization mutations as also described herein, and the two long parallel rectangles with one having a protrusion and the other having an indentation are human Fc domains, e.g. from IgG1, from IgG2, or from IgG4 as described herein, with knob-in-hole and/or ionic pair (a/k/a charged pairs, ionic bond, or charged residue pair) mutations as described herein and optionally with effector knock-out and/or stabilization mutations as also described herein.

FIGS. 1A-F show illustrative homodimeric 2-chain complexes. These figures show illustrative configurations for the homodimeric 2-chain complexes.

FIGS. 2A-H show illustrative homodimeric 2-chain complexes with two targeting moieties (TM) (as described herein, more targeting moieties may be present in some embodiments). In embodiments, the position of TM1 and TM2 are interchangeable. In embodiments, the constructs shown in the box (i.e., FIGS. 2G and 2H) have signaling agent (SA) between TM1 and TM2 or between TM1 and Fc.

FIGS. 3A-H show illustrative homodimeric 2-chain complexes with two signaling agents (as described herein, more signaling agents may be present in some embodiments). In embodiments, the position of SA1 and SA2 are interchangeable. In embodiments, the constructs shown in the box (i.e., FIGS. 3G and 3H) have TM between SA1 and SA2 or TM at N- or C-terminus.

FIGS. 4A-D show illustrative heterodimeric 2-chain complexes with split TM and SA chains, namely the TM on the knob chain of the Fc and the SA on hole chain of the Fc.

FIGS. 5A-F show illustrative heterodimeric 2-chain complexes with split TM and SA chains, namely with both TMs on the knob chain of the Fc and with SA on hole chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 6A-J show illustrative heterodimeric 2-chain complexes with split TM and SA chains, namely with TM on the knob chain of the Fc and with a SA on the hole chain of the Fc, with two signaling agents (as described herein, more signaling agents may be present in some embodiments). In these orientations and/or configurations, one SA is on the knob chain and one SA is on the hole chain. In embodiments, the position of SA1 and SA2 are interchangeable.

FIGS. 7A-D show illustrative heterodimeric 2-chain complexes with split TM and SA chains, namely the SA on the knob chain of the Fc and the TM on hole chain of the Fc.

FIGS. 8A-F show illustrative heterodimeric 2-chain complexes with split TM and SA chains, namely with SA on the knob chain of the Fc and both TMs on hole chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 9A-J show illustrative heterodimeric 2-chain complexes with split TM and SA chains, namely with SA on the knob chain of the Fc and TM on hole chain of the Fc, with two signaling agents (as described herein, more signaling agents may be present in some embodiments). In these orientations and/or configurations, one SA is on the knob chain and one SA is on the hole chain. In embodiments, the position of SA1 and SA2 are interchangeable.

FIGS. 10A-F show illustrative heterodimeric 2-chain complexes with TM and SA on the same chain, namely the SA and TM both on the knob chain of the Fc.

FIGS. 11A-L show illustrative heterodimeric 2-chain complexes with a TM and a SA on the same chain, namely with SA and with TM both on the knob chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 12A-L show illustrative heterodimeric 2-chain complexes with a TM and a SA on the same chain, namely with SA and with TM both on the knob chain of the Fc, with two signaling agents (as described herein, more signaling agents may be present in some embodiments). In embodiments, the position of SA1 and SA2 are interchangeable.

FIGS. 13A-F show illustrative heterodimeric 2-chain complexes with TM and SA on the same chain, namely the SA and TM both on the hole chain of the Fc.

FIGS. 14A-L show illustrative heterodimeric 2-chain complexes with a TM and a SA on the same chain, namely with SA and with TM both on the hole chain of the Fc, with two targeting moieties (as described herein, more targeting moieties are present in some embodiments). In embodiments, the position of TM1 and TM2 are interchangeable. In embodiments, TM1 and TM2 can be identical.

FIGS. 15A-L show illustrative heterodimeric 2-chain complexes with a TM and a SA on the same chain, namely with SA and with TM both on the hole chain of the Fc, with two signaling agents (as described herein, more signaling agents may be present in some embodiments). In embodiments, the position of SA1 and SA2 are interchangeable.

FIGS. 16A-J show illustrative heterodimeric 2-chain complexes with two targeting moieties (as described herein, more targeting moieties may be present in some embodiments) and with SA on knob Fc and TM on each chain. In embodiments, TM1 and TM2 can be identical.

FIGS. 17A-J show illustrative heterodimeric 2-chain complexes with two targeting moieties (as described herein, more targeting moieties may be present in some embodiments) and with SA on hole Fc and TM on each chain. In embodiments, TM1 and TM2 can be identical.

FIGS. 18A-F show illustrative heterodimeric 2-chain complexes with two signaling agents (as described herein, more signaling agents may be present in some embodiments) and with split SA and TM chains: SA on knob and TM on hole Fc.

FIGS. 19A-F show illustrative heterodimeric 2-chain complexes with two signaling agents (as described herein, more signaling agents may be present in some embodiments) and with split SA and TM chains: TM on knob and SA on hole Fc.

FIG. 20 shows pSTAT1 upon FMS-like tyrosine kinase 3 ligand (FLT3L)-Fc-AFN in transient transfected Hek293T cells. FLT3 or MOCK (empty vector) transfected Hek293T cells were stimulated as indicated and stained for pSTAT1. Average % of pSTAT1 positive cells (±STDEV) of duplicate measurements are plotted.

FIGS. 21A-F show some embodiments of split dimeric FLT3-L (TM): formats with AFN (SA) on knob Fc. In some embodiments, the knob in to hole format may be replaced or supplemented with ion charge pair mutations.

FIGS. 22A-F show some embodiments of split dimeric FLT3-L (TM): formats with AFN (SA) on hole Fc. In some embodiments, the knob in to hole format may be replaced or supplemented with ion charge pair mutations.

FIGS. 23A-G show biological activity of FLT3L AFNs on the HL116 reporter. HL116 or HL116-hFLT3 cells were stimulated for 6 hours with serial dilution wild type IFNα2 or FLT3L AFNs. Average luciferase activities (±STDEV) are plotted.

FIGS. 24A-H show single chain dimeric FLT3-L (TM): AFN (SA) formats. The knob into hole may be replaced or supplemented with ion-charge pair mutations.

FIGS. 25A-L show single chain dimeric FLT3-L (TM): AFN (SA) formats. The knob into hole may be replaced or supplemented with ion-charge pair mutations.

FIGS. 26A-B show biological activity of single chain FLT3L AFNs on the HL116 reporter. HL116 or HL116-hFLT3 cells were stimulated for 6 hours with serial dilution wild type IFNα2 or FLT3L AFNs. Average luciferase activities (±STDEV) are plotted.

FIGS. 27A-C show size exclusion chromatography (SEC) profiles of purified split and single chain FLT3L AFNs.

FIG. 28A-B shows tumor growth curves in humanized mice after treatment with buffer or scFlt3-Fc (A) and buffer or scFlt3L-Fc-AFN (B). Average values (in mm³) of 3 to 5 animals per time point time (+SEM) are plotted.

FIG. 29 shows changes in body weight as of day 12 of humanized mice after treatment with buffer, scFlt3-Fc or scFlt3L-Fc-AFN. Average values (in mm³) of 3 to 4 animals per time point time (+SEM) are plotted. Animals that were euthanized before day 26 were excluded.

FIG. 30 shows size exclusion chromatography (SEC) profiles of the purified construct of SEQ ID NO: 73—a monomeric form of Flt3L linked to interferon via a flexible linker.

FIG. 31 shows tumor growth curves in humanized mice after treatment with buffer or Flt3L-IFNα1. Average values (in mm³) of 5 or 6 animals per time point time (+SEM) are plotted.

### DETAILED DESCRIPTION

In some aspects, an chimeric protein or chimeric protein complex, such as an Fc-based chimeric protein complex is provided that comprises a targeting moiety, the targeting moiety be able to specifically bind to an antigen or receptor of interest, the antigen or receptor of interest being FMS-like tyrosine kinase 3 (FLT3). The chimeric protein or chimeric protein complex, such as an Fc-based chimeric protein complex also comprises a wild type signaling agent or a modified form thereof, the signaling agent signaling being one of those described herein, for instance, without limitation, human IFNα2, IFNα1, IFNβ, and IL-1β, which, in various embodiments, can be wild type human or a mutant form.

The present technology is based, in part, on the discovery of signaling agents, that are optionally modified to have reduced affinity or activity for one or more of its receptor(s), and the discovery, engineering and integration of targeting moieties that recognize and bind to a specific target, including FLT3. In some embodiments, the chimeric protein and/or chimeric protein complexes comprise a FLT3L, such as the ECD (extracellular domain) of FLT3L, or portion or variant thereof. In some embodiments the FLT3L-ECD is present in two copies on the same polypeptide (a single chain dimeric FLT3L construct). In some embodiments the FLT3L-ECD is present in a single copy on each of two different polypeptides that can otherwise dimerize to form a chimeric protein complex, such as mediated by Fc-chains in a Fc-based chimeric protein complex. In some embodiments the FLT3L is a FLT3L-ECD, or a portion or variant thereof, that optionally contains a mutation that reduces intermolecular FLT3L-ECD homodimerization (i.e., dimerization of FLT3L domains on separate FLT3L-ECD containing molecules that would not otherwise readily dimerize by other mechanisms), and favoring intramolecular FLT3L-ECD dimerization (i.e., dimerization of two copies of FLT3L-ECDs contained within the same, single polypeptide, i.e., a single chain dimeric FLT3L construct), or dimerization of single FLT3-ECDs on two different polypeptides that can otherwise dimerize within a chimeric protein complex, such as an Fc-based chimeric protein complex. In some embodiments, the mutation in the FLT3L-ECD is L27D (with reference to any one of SEQ ID NOs: 2-4 or L24D with regard to SEQ ID NO: 5). In some embodiments, mutation L27D (with reference to any one of SEQ ID NOs: 2-4 or L24D with regard to SEQ ID NO: 5), or functionally similar mutations in the FLT3-ECD, favor the formation of more homogenous forms of chimeric proteins and chimeric protein complexes, avoiding formation of undesired and higher molecular weight complexes and/or aggregates that would be substantially detrimental to scale up production of the FLT3-targeted construct and in vivo safety of such constructs (e.g., risk of immunoreactivity, loss of activity etc.).

The discovery and use of single chain dimeric FLT3L (e.g., two copies of FLT3L-ECD in a single polypeptide), optionally a FLT3-ECD with a L27D mutation (with reference to any one of SEQ ID NOs: 2-4 or L24D with regard to SEQ ID NO: 5), or a ECD domain mutation with similar or equivalent functional effect, in chimeric proteins and chimeric protein complexes, without wishing to be bound by theory, allows for significant simplification of constructs with FLT3 targeting properties, including, but no limited, to a) retention of functionality, specificity and selectivity of the signaling agent activity, b) FLT3 activation or binding, c) generation of more homogenous preparations of chimeric proteins and chimeric protein complexes due to avoidance of undesired FLT3L-driven intermolecular dimerization of chimeric proteins, c) reduction in size of final product, d) simplification of product characteristics that impact purification and scale up production of such chimeric proteins and chimeric protein complexes, and e) reduced aggregation potential and, accordingly, increased safety properties.

Similar advantages as described for single chain dimeric FLT3L/FLT3L-ECD chimeric protein and chimeric protein complexes, were also found with chimeric protein complexes, such as Fc-based chimeric protein complexes, in which a single copy of a FLT3L-ECD, or portion thereof, is present in a single polypeptide that can otherwise dimerize with another polypeptide that also comprises a single copy of a FLT3-ECD, and for which, in each of the two pairing polypeptides, the optionally FLT3-ECD has a mutation that reduces undesired intermolecular ECD homodimerization, such a L27D (with reference to any one of SEQ ID NOs: 2-4 or L24D with regard to SEQ ID NO: 5), or a mutation(s) that has a functionally similar effect in reducing intermolecular ECD homodimerization. This is exemplified by Fc-based chimeric protein complexes in which each of the two pairing/dimerizing Fc-chains comprises a single copy of FLT3L-ECD, or portion thereof, incorporating such mutation, such as L27D (with reference to any one of SEQ ID NOs: 2-4 or L24D with regard to SEQ ID NO: 5). Complex formation through Fc-chain pairing promotes FLT3L-ECD-mutant (e.g. L27D with reference to any one of SEQ ID NOs: 2-4 or L24D with regard to SEQ ID NO: 5) dimerization and restoration of FLT3 binding activity for such “split FLT3L-ECD” construct, while avoiding high molecular weight and heterogeneous complex formation that is otherwise observed with FLT3L-ECD domains that are not mutated to reduce undesired intermolecular ECD dimerization between Fc-chains and Fc-chimeric complexes. Accordingly, engineering of such chimeric protein complexes allow for significant simplification of constructs with FLT3 targeting properties, including, but no limited, to a) retention of functionality, specificity and selectivity of the signaling agent activity, b) FLT3 activation or binding, c) generation of more homogenous preparations of chimeric protein complexes due to avoidance of undesired FLT3L-driven intermolecular dimerization of chimeric proteins or protein complexes, c) reduction in size of final product, d) simplification of product characteristics that impact purification and scale up production of such chimeric protein complexes, e) reduced aggregation potential and, accordingly, increased safety properties.

In some embodiments, one or more targeting moieties are incorporated in the chimeric proteins or chimeric protein complexes, including Fc-based chimeric protein complexes. In some embodiments, one or more signaling agents and one or more targeting moieties are conjugated to a chimeric protein, or conjugated to Fc domains in context of a Fc-based chimeric protein complex. Such Fc-based chimeric protein complexes, surprisingly, have dramatically extended half-lives in vivo, as compared to other chimeric proteins and/or targeted signaling agent chimeric proteins, especially in the Fc-heterodimer configuration, as described herein, and are particularly amendable to production and purification with standardized methods. Accordingly, the present Fc-based chimeric protein complex approach yields agents that are particularly well suited for use in various therapies, in particular therapies that benefit from intermittent dosing at longer time intervals.

In some embodiments, the targeting moiety in a chimeric protein or chimeric protein complex comprises FLT3L or a portion thereof. In other embodiments, the targeting moiety comprises the extracellular domain of FLT3L, or a portion thereof. In some embodiments, the targeting moiety comprises a functional fragment of the amino acid sequence of SEQ ID NO: 1, e.g., a deletion of about 110 residues, or about 100 residues, or about 90 residues, or about 80 residues, or about 70 residues, or about 60 residues, or about 50 residues, or about 40 residues, or about 30 residues, or about 20 residues, or about 10 residues.

The amino acid sequence for SEQ ID NO: 1 (Flt3L full length) is:

MTVLAPAWSPTTYLLLLLLLSSGLSGTQDCSFQHSPISSDFAVKIREL
SDYLLQDYPVTVASNLQDEELCGGLWRLVLAQRWMERLKTVAGSKM
QGLLERVNTEIHFVTKCAFQPPPSCLRFVQTNISRLLQETSEQLVALKP
WITRQNFSRCLELQCQPDSSTLPPPWSPRPLEATAPTAPQPPLLLLLL
LPVGLLLLAAAWCLHWQRTRRRTPRPGEQVPPVPSPQDLLLVEH

where Bold=leader sequence, Underlined: extracellular region not part of receptor binding domain, Italic=transmembrane and intracellular domain.

In some embodiments, the targeting moiety comprises an amino acid sequence having at least 90% identity with any one of SEQ ID NOs: 2-5, or an amino acid sequence having at least 95% identity with any one of SEQ ID NOs: 2-5.

The amino acid sequence for SEQ ID NO: 2 (mature Flt3L-ec (extracellular domain)) is:

TQDCSFQHSPISSDFAVKIRELSDYLLQDYPVTVASNLQDEELCGGLW
RLVLAQRWMERLKTVAGSKMQGLLERVNTEIHFVTKCAFQPPPSCLR
FVQTNISRLLQETSEQLVALKPWITRQNFSRCLELQCQPDSSTLPPPW
SPRPLEATAPTAPQP.

The amino acid sequence for SEQ ID NO: 3 (mature Flt3L-ec (extracellular domain) function shorter variant commercial source (Prospecbio)) is:

TQDCSFQHSPISSDFAVKIRELSDYLLQDYPVTVASNLQDEELCGGLW
RLVLAQRWMERLKTVAGSKMQGLLERVNTEIHFVTKCAFQPPPSCLR
FVQTNISRLLQETSEQLVALKPWITRQNFSRCLELQCQPDSSTLPPPW
SPRPLEATAPTA.

The amino acid sequence for SEQ ID NO: 4 mature Flt3L-ec (extracellular domain) minimal functional domain (Savvides et al., 2000, Nature Structural Biology) is:

TQDCSFQHSPISSDFAVKIRELSDYLLQDYPVTVASNLQDEELCGGLW
RLVLAQRWMERLKTVAGSKMQGLLERVNTEIHFVTKCAFQPPPSCLR
FVQTNISRLLQETSEQLVALKPWITRQNFSRCLELQCQP.

The amino acid sequence for SEQ ID NO: 5 mature Flt3L-ec (extracellular domain) minimal functional domain (Savvides et al., 2000, Nature Structural Biology) shortened by starting at the first cysteine and ending at the last cysteine is:

CSFQHSPISSDFAVKIRELSDYLLQDYPVTVASNLQDEELCGGLWRLV
LAQRWMERLKTVAGSKMQGLLERVNTEIHFVTKCAFQPPPSCLRFVQ
TNISRLLQETSEQLVALKPWITRQNFSRCLELQC.

In some embodiments, the chimeric protein or chimeric protein complex has one or more targeting moieties, including a FLT3 targeting moiety. In some embodiments, a targeting moiety may be attached to or linked via a linker to the FLT3 targeting moiety or the signaling agent in a chimeric protein. In some embodiments the FLT3 targeting moiety is a single chain dimeric FLT3L, or portion thereof, such as a FLT3-ECD (extracellular domain). In some embodiments, in a chimeric protein complex, the FLT3 targeting moiety, e.g., FLT3-ECD, or variant or fragment thereof, is present on either the same or different polypeptide as the signaling agent.

In embodiments, the invention provides a FLT3L domain which is a single chain dimer of the formula A-B-C, wherein:
A is an amino acid sequence having at least 90% identity, or having at least 95% identity, or having at least 97% identity, or having at least 98% identity, or having at least 99% identity with any one of SEQ ID NOs: 2-5,
B is a flexible linker which is substantially comprised of glycine and serine residues, optionally wherein the flexible linker comprises (Gly₄Ser)ₙ, where n is from about 1 to about 8, optionally wherein the flexible linker comprises one or more of SEQ ID NO: 10-SEQ ID NO: 17, and
C is an amino acid sequence having at least 90% identity, or having at least 95% identity, or having at least 97% identity, or having at least 98% identity, or having at least 99% identity with any one of SEQ ID NOs: 2-5.

The chimeric protein or chimeric protein complex, such as an Fc-based chimeric protein complex in accordance with embodiments of the present invention also comprises a signaling agent or a modified form thereof, the signaling agent as described herein, for instance, without limitation, human IFNα2, IFNα1, IFNβ, and IL-1β. In embodiments, the chimeric protein or chimeric protein complex, such as an Fc-based chimeric protein complex also comprises one or more linkers.

In some embodiments, the chimeric protein or chimeric protein complex, such as an Fc-based chimeric protein complex is such that it has one targeting moiety attached to each Fc chain of the Fc domain. In some embodiments, the chimeric protein or chimeric protein complex, such as an Fc-based chimeric protein complex comprises two targeting moieties attached to one Fc chain of the Fc domain. In some embodiments, the chimeric protein or chimeric protein complex, such as an Fc-based chimeric protein complex is such that the two targeting moieties are attached to each other, optionally via a linker. In some embodiments, the chimeric protein or chimeric protein complex, such as an Fc-based chimeric protein complex is such that the two targeting moieties are attached to the Fc chain, optionally via a linker.

In some embodiments, the chimeric protein or chimeric protein complex, such as an Fc-based chimeric protein complex comprises a polypeptide having an amino acid sequence having at least 95%, or at least 98%, or at least 99% identity with any one of SEQ ID NOs: 40, 41, and 46-57. In embodiments, the chimeric protein or chimeric protein complex, such as an Fc-based chimeric protein complex comprises a polypeptide having an amino acid sequence selected from SEQ ID NOs: 40, 41, and 46-57 and less than 10 mutations to the amino acid sequence. In some embodiments, the chimeric protein or chimeric protein complex, such as an Fc-based chimeric protein complex comprises a polypeptide having an amino acid sequence selected from SEQ ID NOs: 40, 41, and 46-57, and less than 5 mutations to the amino acid sequence. In some embodiments, the chimeric protein or chimeric protein complex, such as an Fc-based chimeric protein complex comprises a polypeptide having an amino acid sequence selected from SEQ ID NOs: 40, 41, and 46-66.

In some embodiments, the chimeric protein or chimeric protein complex, such as an Fc-based chimeric protein complex comprises a first amino acid sequence having at least 95%, or at least 98%, or at least 99% identity to SEQ ID NO: 40 and a second amino acid sequence having at least 95%, or at least 98%, or at least 99% identity to SEQ ID NO: 41.

In embodiments, the chimeric protein or chimeric protein complex, such as an Fc-based chimeric protein complex comprises a first amino acid sequence having at least 95%, or at least 98%, or at least 99% identity to SEQ ID NO: 46 and a second amino acid sequence having at least 95%, or at least 98%, or at least 99% identity to SEQ ID NO: 47.

In some embodiments, the chimeric protein or chimeric protein complex, such as an Fc-based chimeric protein complex comprises a first amino acid sequence having at least 95%, or at least 98%, or at least 99% identity to SEQ ID NO: 48 and a second amino acid sequence having at least 95%, or at least 98%, or at least 99% identity to SEQ ID NO: 49.

In some embodiments, the chimeric protein or chimeric protein complex, such as an Fc-based chimeric protein complex comprises a first amino acid sequence having at least 95%, or at least 98%, or at least 99% identity to SEQ ID NO: 50 and a second amino acid sequence having at least 95%, or at least 98%, or at least 99% identity to SEQ ID NO: 51.

In some embodiments, the chimeric protein or chimeric protein complex, such as an Fc-based chimeric protein complex comprises a first amino acid sequence having at least 95%, or at least 98%, or at least 99% identity to SEQ ID NO: 52 and a second amino acid sequence having at least 95%, or at least 98%, or at least 99% identity to SEQ ID NO: 53.

In some embodiments, the chimeric protein or chimeric protein complex, such as an Fc-based chimeric protein complex comprises a first amino acid sequence having at least 95%, or at least 98%, or at least 99% identity to SEQ ID NO: 54 and a second amino acid sequence having at least 95%, or at least 98%, or at least 99% identity to SEQ ID NO: 55.

In some embodiments, the chimeric protein or chimeric protein complex, such as an Fc-based chimeric protein complex comprises a first amino acid sequence having at least 95%, or at least 98%, or at least 99% identity to SEQ ID NO: 56 and a second amino acid sequence having at least 95%, or at least 98%, or at least 99% identity to SEQ ID NO: 57.

In some embodiments, the chimeric protein or chimeric protein complex, such as an Fc-based chimeric protein complex comprises a first amino acid sequence having at least 95%, or at least 98%, or at least 99% identity to SEQ ID NO: 58 and a second amino acid sequence having at least 95%, or at least 98%, or at least 99% identity to SEQ ID NO: 59.

In some embodiments, the chimeric protein or chimeric protein complex, such as an Fc-based chimeric protein complex comprises a first amino acid sequence having at least 95%, or at least 98%, or at least 99% identity to any one of sequences selected from SEQ ID NOs: 60-65 and a second amino acid sequence having at least 95%, or at least 98%, or at least 99% identity to SEQ ID NO: 66.

In some embodiments, the targeting moiety of the present invention is a single chain FLT3L AFNs with or without Fc domain. In some embodiments, the FLT3L has a L27D mutation (with reference to any one of SEQ ID NOs: 2-4 or L24D with regard to SEQ ID NO: 5) at the dimerization interface. In some embodiments the FLT3L has one or more mutation that reduces homodimerization of FLT3L. In some embodiments the FLT3L has one or more mutations in the FLT3L homodimerization domains (amino acid regions 25-30 and 63-68 with reference to any one of SEQ ID NOs: 2-4) that reduces FLT3L dimerization. In some embodiments, two FLT3L sequences, or modified FLT3L sequences, can be fused together, optionally, by a linker, e.g., a 3*GGGGS linker or any linker disclosed herein. In some embodiments, two or more FLT3L sequences, or modified FLT3L sequences, can be linked to a signaling agent as described herein. In some embodiments, two FLT3L sequences, or modified FLT3L sequences, are linked to a hIFNa sequence (e.g., IFNα1, IFNα2) or a modified form thereof.

### Signaling Agents (SA)

In various embodiments, the signaling agent is a wild type signaling agent. In various embodiments the activity of the wild type signaling agent is attenuated by attachment, linkage or fusion of the signaling agent in a chimeric protein or chimeric protein complex. In various embodiments, a wild type signaling agent is a type I interferon.

In some embodiments, the signaling agent comprises an amino acid sequence having at least 95% identity with one of SEQ ID NO: 6, 7, 38, 39, or 74, or the signaling agent can comprise an amino acid sequence of one of SEQ ID NO: 6, 7, 38, 39, or 74.

In various embodiments, the signaling agent is a modified (e.g., mutant) form of the signaling agent having one or more mutations. In various embodiments, the mutations allow for the modified signaling agent to have one or more of attenuated activity such as one or more of reduced binding affinity, reduced endogenous activity, and reduced specific bioactivity relative to unmodified or unmutated, i.e. the wild type form of the signaling agent (e.g. comparing the same signaling agent in a wild type form versus a modified (e.g. mutant) form). In various embodiments, the mutations allow for the modified signaling agent to have one or more of attenuated activity such as one or more of reduced binding affinity, reduced endogenous activity, and reduced specific bioactivity relative to unmodified or unmutated, e.g. wild type IFNα2, IFNα1, IFNβ, or IL-1β. In some embodiments, the mutations which attenuate or reduce binding or affinity include those mutations which substantially reduce or ablate binding or activity. In some embodiments, the mutations which attenuate or reduce binding or affinity are different than those mutations which substantially reduce or ablate binding or activity. Consequentially, in various embodiments, the mutations allow for the signaling agent to be more safe, e.g. have reduced systemic toxicity, reduced side effects, and reduced off-target effects relative to unmutated, i.e. wild type, signaling agent (e.g. comparing the same signaling agent in a wild type form versus a modified (e.g. mutant) form). In various embodiments, the mutations allow for the signaling agent to be safer, e.g. have reduced systemic toxicity, reduced side effects, and reduced off-target effects relative to unmutated interferon, e.g. the unmutated sequence of IFNα2, IFNα1, IFNβ, or IL-1β.

In various embodiments, the signaling agent is modified to have one or more mutations that reduce its binding affinity or activity for one or more of its receptors. In some embodiments, the signaling agent is modified to have one or more mutations that substantially reduce or ablate binding affinity or activity for the receptors. In some embodiments, the activity provided by the wild type signaling agent is agonism at the receptor (e.g. activation of a cellular effect at a site of therapy). For example, the wild type signaling agent may activate its receptor. In such embodiments, the mutations result in the modified signaling agent to have reduced or ablated activating activity at the receptor. For example, the mutations may result in the modified signaling agent to deliver a reduced activating signal to a target cell or the activating signal could be ablated. In some embodiments, the activity provided by the wild type signaling agent is antagonism at the receptor (e.g. blocking or dampening of a cellular effect at a site of therapy). For example, the wild type signaling agent may antagonize or inhibit the receptor. In these embodiments, the mutations result in the modified signaling agent to have a reduced or ablated antagonizing activity at the receptor. For example, the mutations may result in the modified signaling agent to deliver a reduced inhibitory signal to a target cell or the inhibitory signal could be ablated. In various embodiments, the signaling agent is antagonistic due to one or more mutations, e.g. an agonistic signaling agent is converted to an antagonistic signaling agent (e.g. as described in WO 2015/007520, the entire contents of which are hereby incorporated by reference) and, such a converted signaling agent, optionally, also bears one or more mutations that reduce its binding affinity or activity for one or more of its receptors or that substantially reduce or ablate binding affinity or activity for one or more of its receptors.

In some embodiments, the reduced affinity or activity at the receptor is restorable by attachment with one or more of the targeting moieties or upon inclusion in an chimeric protein or chimeric protein complex, such as an Fc-based chimeric protein complex as disclosed herein. In other embodiments, the reduced affinity or activity at the receptor is not substantially restorable by the activity of one or more of the targeting moieties or upon inclusion in an chimeric protein or chimeric protein complex, such as an Fc-based chimeric protein complex as disclosed herein.

In various embodiments, the signaling agent is active on target cells because the targeting moiety compensates for the missing/insufficient binding (e.g., without limitation and/or avidity) required for substantial activation. In various embodiments, the modified signaling agent is substantially inactive en route to the site of therapeutic activity and has its effect substantially on specifically targeted cell types which greatly reduces undesired side effects.

In some embodiments, the signaling agent may include one or more mutations that attenuate or reduce binding or affinity for one receptor (i.e., a therapeutic receptor) and one or more mutations that substantially reduce or ablate binding or activity at a second receptor. In such embodiments, these mutations may be at the same or at different positions (i.e., the same mutation or multiple mutations). In some embodiments, the mutation(s) that reduce binding and/or activity at one receptor is different than the mutation(s) that substantially reduce or ablate at another receptor. In some embodiments, the mutation(s) that reduce binding and/or activity at one receptor is the same as the mutation(s) that substantially reduce or ablate at another receptor. In some embodiments, the present chimeric proteins or chimeric protein complexes, such as an Fc-based chimeric protein complexes have a modified signaling agent that has both mutations that attenuate binding and/or activity at a therapeutic receptor and therefore allow for a more controlled, on-target therapeutic effect (e.g. relative wild type signaling agent) and mutations that substantially reduce or ablate binding and/or activity at another receptor and therefore reduce side effects (e.g. relative to wild type signaling agent).

In some embodiments, the substantial reduction or ablation of binding or activity is not substantially restorable with a targeting moiety or upon inclusion in an chimeric protein or chimeric protein complex, such as an Fc-based chimeric protein complex as disclosed herein. In some embodiments, the substantial reduction or ablation of binding or activity is restorable with a targeting moiety or upon inclusion in an chimeric protein or chimeric protein complex, such as an Fc-based chimeric protein complex as disclosed herein. In various embodiments, substantially reducing or ablating binding or activity at a second receptor also may prevent deleterious effects that are mediated by the other receptor. Alternatively, or in addition, substantially reducing or ablating binding or activity at the other receptor causes the therapeutic effect to improve as there is a reduced or eliminated sequestration of the therapeutic chimeric proteins or chimeric protein complexes, such as an Fc-based chimeric protein complexes away from the site of therapeutic action. For instance, in some embodiments, this obviates the need of high doses of the present chimeric proteins or chimeric protein complexes, such as an Fc-based chimeric protein complexes that compensate for loss at the other receptor. Such ability to reduce dose further provides a lower likelihood of side effects.

In various embodiments, the modified signaling agent comprises one or more mutations that cause the signaling agent to have reduced, substantially reduced, or ablated affinity, e.g. binding (e.g. K_{D}) and/or activation (for instance, when the modified signaling agent is an agonist of its receptor, measurable as, for example, K_{A} and/or EC₅₀) and/or inhibition (for instance, when the modified signaling agent is an antagonist of its receptor, measurable as, for example, K_{I} and/or IC₅₀), for one or more of its receptors. In various embodiments, the reduced affinity at the signaling agent's receptor allows for attenuation of activity (inclusive of agonism or antagonism). In such embodiments, the modified signaling agent has about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or about 10%-20%, about 20%-40%, about 50%, about 40%-60%, about 60%-80%, about 80%-100% of the affinity for the receptor relative to the wild type signaling agent. In some embodiments, the binding affinity is at least about 2-fold lower, about 3-fold lower, about 4-fold lower, about 5-fold lower, about 6-fold lower, about 7-fold lower, about 8-fold lower, about 9-fold lower, at least about 10-fold lower, at least about 15-fold lower, at least about 20-fold lower, at least about 25-fold lower, at least about 30-fold lower, at least about 35-fold lower, at least about 40-fold lower, at least about 45-fold lower, at least about 50-fold lower, at least about 100-fold lower, at least about 150-fold lower, or about 10-50-fold lower, about 50-100-fold lower, about 100-150-fold lower, about 150-200-fold lower, or more than 200-fold lower relative to the wild type signaling agent (including, by way of non-limitation, relative to the unmutated IFNα2, IFNα1, IFNβ, or IL-1β).

In embodiments wherein the chimeric protein or chimeric protein complex, such as an Fc-based chimeric protein complex has mutations that reduce binding at one receptor and substantially reduce or ablate binding at a second receptor, the attenuation or reduction in binding affinity of a modified signaling agent for one receptor is less than the substantial reduction or ablation in affinity for the other receptor. In some embodiments, the attenuation or reduction in binding affinity of a modified signaling agent for one receptor is less than the substantial reduction or ablation in affinity for the other receptor by about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, or about 95%. In various embodiments, substantial reduction or ablation refers to a greater reduction in binding affinity and/or activity than attenuation or reduction.

In various embodiments, the modified signaling agent comprises one or more mutations that reduce the endogenous activity of the signaling agent to about 75%, or about 70%, or about 60%, or about 50%, or about 40%, or about 30%, or about 25%, or about 20%, or about 10%, or about 5%, or about 3%, or about 1%, e.g., relative to the wild type signaling agent (including, by way of non-limitation, relative to the unmutated IFNα2, IFNα1, IFNβ, or IL-1β).

In various embodiments, the modified signaling agent comprises one or more mutations that cause the signaling agent to have reduced affinity and/or activity for a receptor of any one of the cytokines, growth factors, and hormones as described herein.

In some embodiments, the modified signaling agent comprises one or more mutations that cause the signaling agent to have reduced affinity for its receptor that is lower than the binding affinity of the targeting moiety for its(their) receptor(s). In some embodiments, this binding affinity differential is between signaling agent/receptor and targeting moiety/receptor on the same cell. In some embodiments, this binding affinity differential allows for the signaling agent, e.g. mutated signaling agent, to have localized, on-target effects and to minimize off-target effects that underlie side effects that are observed with wild type signaling agent. In some embodiments, this binding affinity is at least about 2-fold, or at least about 5-fold, or at least about 10-fold, or at least about 15-fold lower, or at least about 25-fold, or at least about 50-fold lower, or at least about 100-fold, or at least about 150-fold.

Receptor binding activity may be measured using methods known in the art. For example, affinity and/or binding activity may be assessed by Scatchard plot analysis and computer-fitting of binding data (e.g. Scatchard, 1949 Annals of the New York Academy of Sciences. 51 (4): 660-672) or by reflectometric interference spectroscopy under flow through conditions, as described by Brecht et al. (1993), Biosens Bioelectron 1993; 8:387-392 the entire contents of all of which are hereby incorporated by reference.

In various embodiments, the chimeric protein or chimeric protein complex, such as an Fc-based chimeric protein complex comprises a wild type signaling agent that has improved target selectivity and safety relative to a signaling agent which is not fused to an Fc, or a signaling agent which is not in the context of a complex, e.g., without limitation, a heterodimeric complex. In various embodiments, the chimeric protein or chimeric protein complex, such as an Fc-based chimeric protein complex comprises a wild type signaling agent that has improved target selective activity relative to a signaling agent which is not fused to an Fc, or a signaling agent which is not in the context of a complex, e.g., without limitation, a heterodimeric complex. In various embodiments, the chimeric protein or chimeric protein complex, such as an Fc-based chimeric protein complex allows for conditional activity.

In various embodiments, the chimeric protein or chimeric protein complex, such as an Fc-based chimeric protein complex comprises a wild type signaling agent that has improved safety, e.g. reduced systemic toxicity, reduced side effects, and reduced off-target effects relative to a signaling agent which is not fused to an Fc, or a signaling agent which is not in the context of a complex, e.g., without limitation, a heterodimeric complex. In various embodiments, improved safety means that the present Fc-based chimeric protein provides lower toxicity (e.g. systemic toxicity and/or tissue/organ-associated toxicities); and/or lessened or substantially eliminated side effects; and/or increased tolerability, lessened or substantially eliminated adverse events; and/or reduced or substantially eliminated off-target effects; and/or an increased therapeutic window of the wild type signaling agent as compared to the signaling agent which is not fused to an Fc, or a signaling agent which is not in the context of a complex, e.g., without limitation, a heterodimeric complex.

In some embodiments, the reduced affinity or activity at the receptor is restorable by attachment with one or more of the targeting moieties as described herein or upon inclusion in an chimeric protein or chimeric protein complex, such as an Fc-based chimeric protein complex as disclosed herein.

In various embodiments, the chimeric protein or chimeric protein complex, such as an Fc-based chimeric protein complex comprises a wild type signaling agent that has reduced, substantially reduced, or ablated affinity, e.g. binding (e.g. K_{D}) and/or activation (for instance, when the modified signaling agent is an agonist of its receptor, measurable as, for example, K_{A} and/or EC₅₀) and/or inhibition (for instance, when the modified signaling agent is an antagonist of its receptor, measurable as, for example, K_{I} and/or IC₅₀), for one or more of its receptors. In various embodiments, the reduced affinity at the signaling agent's receptor allows for attenuation of activity. In such embodiments, the modified signaling agent has about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or about 10%-20%, about 20%-40%, about 50%, about 40%-60%, about 60%-80%, about 80%-100% of the affinity for the receptor as compared to the signaling agent which is not fused to an Fc, or a signaling agent which is not in the context of a complex, e.g., without limitation, a heterodimeric complex. In some embodiments, the binding affinity is at least about 2-fold lower, about 3-fold lower, about 4-fold lower, about 5-fold lower, about 6-fold lower, about 7-fold lower, about 8-fold lower, about 9-fold lower, at least about 10-fold lower, at least about 15-fold lower, at least about 20-fold lower, at least about 25-fold lower, at least about 30-fold lower, at least about 35-fold lower, at least about 40-fold lower, at least about 45-fold lower, at least about 50-fold lower, at least about 100-fold lower, at least about 150-fold lower, or about 10-50-fold lower, about 50-100-fold lower, about 100-150-fold lower, about 150-200-fold lower, or more than 200-fold lower as compared to the signaling agent which is not fused to an Fc, or a signaling agent which is not in the context of a complex, e.g., without limitation, a heterodimeric complex.

In various embodiments, the chimeric protein or chimeric protein complex, such as an Fc-based chimeric protein complex comprises a wild type signaling agent that has reduced endogenous activity of the signaling agent to about 75%, or about 70%, or about 60%, or about 50%, or about 40%, or about 30%, or about 25%, or about 20%, or about 10%, or about 5%, or about 3%, or about 1%, e.g., as compared to the signaling agent which is not fused to an Fc, or a signaling agent which is not in the context of a complex, e.g., without limitation, a heterodimeric complex.

In embodiments, the wild type or modified signaling agent is an interferon is a type I interferon. In embodiments, the wild type or modified signaling agent is selected from IFNα2, IFNα1, IFN-α1, IFN-β, IFN-γ, Consensus IFN, IFN-ε, IFN-κ, IFN-τ, IFN-δ, and IFN-v.

In embodiments, the wild type or modified signaling agent is interferon α. In such embodiments, the modified IFNα2 agent has reduced affinity and/or activity for the IFN-α/β receptor (IFNAR), i.e., IFNAR1 and/or IFNAR2 chains. In some embodiments, the modified IFNα2 agent has substantially reduced or ablated affinity and/or activity for the IFN-α/β receptor (IFNAR), i.e., IFNAR1 and/or IFNAR2 chains.

Mutant forms of interferon α2 are known to the person skilled in the art. In an illustrative embodiment, the modified signaling agent is the allelic form IFNα2a having the amino acid sequence of:

(SEQ ID NO: 6)
CDLPQTHSLGSRRTLMLLAQMRKISLFSCLKDRHDFGFPQEEFGNQFQ
KAETIPVLHEMIQQIFNLFSTKDSSAAWDETLLDKFYTELYQQLNDLE
ACVIQGVGVTETPLMKEDSILAVRKYFQRITLYLKEKKYSPCAWEVVR
AEIMRSFSLSTNLQESLRSKE.

In an illustrative embodiment, the wild type or modified signaling agent is the allelic form IFNα2b having the amino acid sequence of:

CDLPQTHSLGSRRTLMLLAQMRRISLFSCLKDRHDFGFPQEEFGNQFQ
KAETIPVLHEMIQQIFNLFSTKDSSAAWDETLLDKFYTELYQQLNDLE
ACVIQGVGVTETPLMKEDSILAVRKYFQRITLYLKEKKYSPCAWEWRA
EIMRSFSLSTNLQESLRSKE (SEQ ID NO: 7; which
differs from IFNα2a at amino acid position 23).

In some embodiments, said IFNα2 mutant (IFNα2a or IFNα2b) is mutated at one or more amino acids at positions 144-154, such as amino acid positions 148, 149 and/or 153. In some embodiments, the IFNα2 mutant comprises one or more mutations selected from L153A, R149A, and M148A. Such mutants are described, for example, in WO2013/107791 and Piehler et al., (2000) J. Biol. Chem, 275:40425-33, the entire contents of all of which are hereby incorporated by reference.

In some embodiments, the IFNα2 mutants have reduced affinity and/or activity for IFNAR1. In some embodiments, the IFNα2 mutant comprises one or more mutations selected from F64A, N65A, T69A, L80A, Y85A, and Y89A, as described in WO2010/030671, the entire contents of which is hereby incorporated by reference.

In some embodiments, the IFNα2 mutant comprises one or more mutations selected from K133A, R144A, R149A, and L153A as described in WO2008/124086, the entire contents of which is hereby incorporated by reference. In some embodiments, the IFNα2 mutant comprises one or more mutations selected from R120E and R120E/K121E, as described in WO2015/007520 and WO2010/030671, the entire contents of which are hereby incorporated by reference. In such embodiments, said IFNα2 mutant antagonizes wildtype IFNα2 activity. In such embodiments, said mutant IFNα2 has reduced affinity and/or activity for IFNAR1 while affinity and/or activity of IFNR2 is retained.

In some embodiments, the human IFNα2 mutant comprises (1) one or more mutations selected from R120E and R120E/K121E, which, without wishing to be bound by theory, create an antagonistic effect and (2) one or more mutations selected from K133A, R144A, R149A, and L153A, which, without wishing to be bound by theory, allow for an attenuated effect at, for example, IFNAR2. In an embodiment, the human IFNα2 mutant comprises R120E and L153A.

In some embodiments, the human IFNα2 mutant comprises one or more mutations selected from, L15A, A19W, R22A, R23A, L26A, F27A, L30A, L30V, K31A, D32A, R33K, R33A, R33Q, H34A, D35A, Q40A, T106A, T106E, D114R, L117A, R120A, R125A, K134A, R144A, A145G, A145M, M148A, R149A, S152A, L153A, and N156A as disclosed in WO 2013/059885, the entire disclosures of which are hereby incorporated by reference. In some embodiments, the human IFNα2 mutant comprises the mutations H57Y, E58N, Q61S, and/or L30A as disclosed in WO 2013/059885. In some embodiments, the human IFNα2 mutant comprises the mutations H57Y, E58N, Q61S, and/or R33A as disclosed in WO 2013/059885. In some embodiments, the human IFNα2 mutant comprises the mutations H57Y, E58N, Q61S, and/or M148A as disclosed in WO 2013/059885. In some embodiments, the human IFNα2 mutant comprises the mutations H57Y, E58N, Q61S, and/or L153A as disclosed in WO 2013/059885. In some embodiments, the human IFNα2 mutant comprises the mutations N65A, L80A, Y85A, and/or Y89A as disclosed in WO 2013/059885. In some embodiments, the human IFNα2 mutant comprises the mutations N65A, L80A, Y85A, Y89A, and/or D114A as disclosed in WO 2013/059885.

In various embodiments, the signaling agent is a mutant human IFNα2. In some embodiments, the mutant human IFNα2 comprises an amino acid sequence having at least 95% identity with SEQ ID NO: 6 or 7, wherein the mutant human IFNα2 has one or more mutations that confer improved safety as compared to a wild type IFNα2 having an amino acid sequence of SEQ ID NO: 6 or 7. In some embodiments, the IFNα2 has one or more mutations at positions 144 to 154 with respect to SEQ ID NO: 6 or 7. In some embodiments, the human IFNα2 has one or more mutations at positions L15, A19, R22, R23, L26, F27, L30, L30, K31, D32, R33, H34, D35, Q40, H57, E58, Q61, F64, N65, T69, L80, Y85, Y89, D114, L117, R120, R125, K133, K134, R144, A145, M148, R149, S152, L153, and N156 with respect to SEQ ID NO: 6 or 7. In some embodiments, the mutant IFNα2 has one or more mutations at position R149, M148, or L153 with respect to SEQ ID NO: 6 or 7. In some embodiments, the one or more mutations are one or more of L15A, A19W, R22A, R23A, L26A, F27A, L30A, L30V, K31A, D32A, R33K, R33A, R33Q, H34A, D35A, Q40A, H57Y, E58N, Q61S, F64A, N65A, T69A, L80A, Y85A, Y89A, D114R, L117A, R120A, R125A, K133A, K134A, R144A, A145G, A145M, M148A, R149A, S152A, L153A, and N156A with respect to SEQ ID NO: 6 or 7. In some embodiments, the mutant human IFNα2 has R149A mutation with respect to SEQ ID NO: 6 or 7.

In some embodiments, the mutant human IFNα2 has one or more mutations at position R33, R144, A145, M148, R149, and L153 with respect to SEQ ID NO: 6 or 7. In some embodiments, the mutant human IFNα2 has a R33A, R144A, R1441, R144L, R144S, R144T, R144Y, A145D, A145G, A145H, A145K, A145Y, M148A, R149A, and L153A mutation with respect to SEQ ID NO: 6 or 7.

In some embodiments, the mutant human IFNα2 has one or more mutations at position R33, T106, R144, A145, M148, R149, and L153 with respect to SEQ ID NO: 6 or 7. In some embodiments, the mutant human IFNα2 has one or more mutations selected from R33A, T106X₃, R120E, R144X₁ A145X₂, M148A, R149A, and L153A with respect to amino acid sequence of SEQ ID NO: 6 or 7, wherein X₁ is selected from A, S, T, Y, L, and I, wherein X₂ is selected from G, H, Y, K, and D, and wherein X₃ is selected from A and E.

In some embodiments, the signaling agent is a wild type interferon α1 or a modified interferon α1. In some embodiments, the present invention provides a chimeric protein or Fc-based chimeric protein complex that includes a wild type IFNα1. In various embodiments, the wild-type IFNα1 comprises the following amino acid sequence:

(SEQ ID NO: 74)
CDLPETHSLDNRRTLMLLAQMSRISPSSCLMDRHDFGFPQEEFDGNQFQ
KAPAISVLHELIQQIFNLFTTKDSSAAWDEDLLDKFCTELYQQLNDLEA
CVMQEERVGETPLMNADSILAVKKYFRRITLYLTEKKYSPCAWEVVRAE
IMRSLSLSTNLQERLRRKE.

In various embodiments, the chimeric protein or Fc-based chimeric protein complex of the invention comprises a modified version of IFNα1, i.e., a IFNα1 variant including a IFNα1 mutant, as a signaling agent. In various embodiments, the IFNα1 variant encompasses mutants, functional derivatives, analogs, precursors, isoforms, splice variants, or fragments of the interferon.

In some embodiments, the IFNα1 interferon is modified to have a mutation at one or more amino acids at positions L15, A19, R23, S25, L30, D32, R33, H34, Q40, C86, D115, L118, K121, R126, E133, K134, K135, R145, A146, M149, R150, S153, L154, and N157 with reference to SEQ ID NO: 74. The mutations can optionally be a hydrophobic mutation and can be, e.g., selected from alanine, valine, leucine, and isoleucine. In some embodiments, the IFNα1 interferon is modified to have a one or more mutations selected from L15A, A19W, R23A, S25A, L30A, L30V, D32A, R33K, R33A, R33Q, H34A, Q40A, C86S, C86A, D115R, L118A, K121A, K121E, R126A, R126E, E133A, K134A, K135A, R145A, R145D, R145E, R145G, R145H, R145I, R145K, R145L, R145N, R145Q, R145S, R145T, R145V, R145Y, A146D, A146E, A146G, A146H, A146I, A146K, A146L, A146M, A146N, A146Q, A146R, A146S, A146T, A146V, A146Y, M149A, M149V, R150A, S153A, L154A, and N157A with reference to SEQ ID NO: 1042. In some embodiments, the IFNα1 mutant comprises one or more multiple mutations selected from L30A/H58Y/E59N_Q62S, R33A/H58Y/E59N/Q62S, M149A/H58Y/E59N/Q62S, L154A/H58Y/E59N/Q62S, R145A/H58Y/E59N/Q62S, D115A/R121A, L118A/R121A, L118A/R121A/K122A, R121A/K122A, and R121E/K122E with reference to SEQ ID NO: 74.

In some embodiments, the IFN-α1 is a variant that comprises one or more mutations which reduce undesired disulphide pairings wherein the one or more mutations are, e.g., at amino acid positions C1, C29, C86, C99, or C139 with reference to SEQ ID NO: 74. In some embodiments, the mutation at position C86 can be, e.g., C86S or C86A or C86Y. These C86 mutants of IFN-α1 are called reduced cysteine-based aggregation mutants. In some embodiment, the IFNα1 variant includes mutations at positions C1, C86 and C99 with reference to SEQ ID NO: 74.

In embodiments, the wild type or modified signaling agent is IFN-β. In some embodiments, the IFN-β is human having a sequence as shown below:

(SEQ ID NO: 38)
MSYNLLGFLQRSSNFQCQKLLWQLNGRLEYCLKDRMNFDIPEEIKQLQQ
FQKEDAALTIYEMLQNIFAIFRQDSSSTGWNETIVENLLANVYHQINHL
KTVLEEKLEKEDFTRGKLMSSLHLKRYYGRILHYLKAKEYSHCAWTIVR
VEILRNFYFINRLTGYLRN.

In various embodiments, the IFN-β encompasses functional derivatives, analogs, precursors, isoforms, splice variants, or fragments of IFN-β. In various embodiments, the IFN-β encompasses IFN-β derived from any species. In an embodiment, chimeric protein or chimeric protein complex, such as an Fc-based chimeric protein complex comprises a modified version of mouse IFN-β. In another embodiment, the chimeric protein or chimeric protein complex, such as an Fc-based chimeric protein complex comprises a modified version of human IFN-β. Human IFN-β is a polypeptide with a molecular weight of about 22 kDa comprising 166 amino acid residues. The amino acid sequence of human IFN-β is SEQ ID NO: 38.

In some embodiments, the human IFN-β is IFN-β-1a which is a glycosylated form of human IFN-β. In some embodiments, the human IFN-β is IFN-β-1b which is a non-glycosylated form of human IFN-β that has a Met-1 deletion and a Cys-17 to Ser mutation.

In various embodiments, the modified IFN-β has one or more mutations that reduce its binding to or its affinity for the IFNAR1 subunit of IFNAR. In one embodiment, the modified IFN-β has reduced affinity and/or activity at IFNAR1. In various embodiments, the modified IFN-β is human IFN-β and has one or more mutations at positions F67, R71, L88, Y92,195, N96, K123, and R124. In some embodiments, the one or more mutations are substitutions selected from F67G, F67S, R71A, L88G, L88S, Y92G, Y92S, 195A, N96G, K123G, and R124G. In an embodiment, the modified IFN-β comprises the F67G mutation. In an embodiment, the modified IFN-β comprises the K123G mutation. In an embodiment, the modified IFN-β comprises the F67G and R71A mutations. In an embodiment, the modified IFN-β comprises the L88G and Y92G mutations. In an embodiment, the modified IFN-β comprises the Y92G, 195A, and N96G mutations. In an embodiment, the modified IFN-β comprises the K123G and R124G mutations. In an embodiment, the modified IFN-β comprises the F67G, L88G, and Y92G mutations. In an embodiment, the modified IFN-β comprises the F67S, L88S, and Y92S mutations.

In some embodiments, the modified IFN-β has one or more mutations that reduce its binding to or its affinity for the IFNAR2 subunit of IFNAR. In one embodiment, the modified IFN-β has reduced affinity and/or activity at IFNAR2. In various embodiments, the modified IFN-β is human IFN-β and has one or more mutations at positions W22, R27, L32, R35, V148, L151, R152, and Y155. In some embodiments, the one or more mutations are substitutions selected from W22G, R27G, L32A, L32G, R35A, R35G, V148G, L151G, R152A, R152G, and Y155G. In an embodiment, the modified IFN-β comprises the W22G mutation. In an embodiment, the modified IFN-β comprises the L32A mutation. In an embodiment, the modified IFN-β comprises the L32G mutation. In an embodiment, the modified IFN-β comprises the R35A mutation. In an embodiment, the modified IFN-β comprises the R35G mutation. In an embodiment, the modified IFN-β comprises the V148G mutation. In an embodiment, the modified IFN-β comprises the R152A mutation. In an embodiment, the modified IFN-β comprises the R152G mutation. In an embodiment, the modified IFN-β comprises the Y155G mutation. In an embodiment, the modified IFN-β comprises the W22G and R27G mutations. In an embodiment, the modified IFN-β comprises the L32A and R35A mutation. In an embodiment, the modified IFN-β comprises the L151G and R152A mutations. In an embodiment, the modified IFN-β comprises the V148G and R152A mutations.

In some embodiments, the modified IFN-β has one or more of the following mutations: R35A, R35T, E42K, M621, G78S, A141Y, A142T, E149K, and R152H. In some embodiments, the modified IFN-β has one or more of the following mutations: R35A, R35T, E42K, M621, G78S, A141Y, A142T, E149K, and R152H in combination with C17S or C17A.

In some embodiments, the modified IFN-β has one or more of the following mutations: R35A, R35T, E42K, M621, G78S, A141Y, A142T, E149K, and R152H in combination with any of the other IFN-β mutations described herein. The crystal structure of human IFN-β is known and is described in Karpusas et al., (1998) PNAS, 94(22): 11813-11818. Specifically, the structure of human IFN-β has been shown to include five α-helices (i.e., A, B, C, D, and E) and four loop regions that connect these helices (i.e., AB, BC, CD, and DE loops). In various embodiments, the modified IFN-β has one or more mutations in the A, B, C, D, E helices and/or the AB, BC, CD, and DE loops which reduce its binding affinity or activity at a therapeutic receptor such as IFNAR. Exemplary mutations are described in WO2000/023114 and US20150011732, the entire contents of which are hereby incorporated by reference. In an exemplary embodiment, the modified IFN-β is human IFN-β comprising alanine substitutions at amino acid positions 15, 16, 18, 19, 22, and/or 23. In an exemplary embodiment, the modified IFN-β is human IFN-β comprising alanine substitutions at amino acid positions 28-30, 32, and 33. In an exemplary embodiment, the modified IFN-β is human IFN-β comprising alanine substitutions at amino acid positions 36, 37, 39, and 42. In an exemplary embodiment, the modified IFN-β is human IFN-β comprising alanine substitutions at amino acid positions 64 and 67 and a serine substitution at position 68. In an exemplary embodiment, the modified IFN-β is human IFN-β comprising alanine substitutions at amino acid positions 71-73. In an exemplary embodiment, the modified IFN-β is human IFN-β comprising alanine substitutions at amino acid positions 92, 96, 99, and 100. In an exemplary embodiment, the modified IFN-β is human IFN-β comprising alanine substitutions at amino acid positions 128, 130, 131, and 134. In an exemplary embodiment, the modified IFN-β is human IFN-β comprising alanine substitutions at amino acid positions 149, 153, 156, and 159.

In some embodiments, the mutant IFNβ comprises SEQ ID NO: 38 and a mutation at W22, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at R27, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at W22, the mutation being an aliphatic hydrophobic residue selected from glycine (G), alanine (A), leucine (L), isoleucine (I), methionine (M), and valine (V) and a mutation at R27, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at L32, the mutation being an aliphatic hydrophobic residue selected from glycine (G), alanine (A), isoleucine (I), methionine (M), and valine (V).

In some embodiments, the mutant IFNβ comprises SEQ ID NO: 38 and a mutation at R35, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at L32, the mutation being an aliphatic hydrophobic residue selected from glycine (G), alanine (A), isoleucine (I), methionine (M), and valine (V) and a mutation at R35, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at F67, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at R71, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at F67, the mutation being an aliphatic hydrophobic residue selected from glycine (G), alanine (A), leucine (L), isoleucine (I), methionine (M), and valine (V) and a mutation at R71, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at L88, the mutation being an aliphatic hydrophobic residue selected from glycine (G), alanine (A), isoleucine (I), methionine (M), and valine (V).

In some embodiments, the mutant IFNβ comprises SEQ ID NO: 38 and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at F67, the mutation being an aliphatic hydrophobic residue selected from glycine (G), alanine (A), leucine (L), isoleucine (I), methionine (M), and valine (V) and a mutation at L88, the mutation being an aliphatic hydrophobic residue selected from glycine (G), alanine (A), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at L88, the mutation being an aliphatic hydrophobic residue selected from glycine (G), alanine (A), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 38 and a mutation at 195, the mutation being an aliphatic hydrophobic residue selected from glycine (G), alanine (A), leucine (L), methionine (M), and valine (V) and a mutation at Y92, the mutation being an aliphatic hydrophobic residue selected from glycine (G), alanine (A), leucine (L), isoleucine (1), methionine (M), and valine (V).

In some embodiments, the mutant IFNβ comprises SEQ ID NO: 38 and a mutation at N96, the mutation being an aliphatic hydrophobic residue selected from glycine (G), alanine (A), leucine (L), isoleucine (1), methionine (M), and valine (V) and a mutation at Y92, the mutation being an aliphatic hydrophobic residue selected from glycine (G), alanine (A), leucine (L), isoleucine (1), methionine (M), and valine (V).

In some embodiments, the mutant IFNβ comprises SEQ ID NO: 38 and a mutation at Y92, the mutation being an aliphatic hydrophobic residue selected from glycine (G), alanine (A), leucine (L), isoleucine (1), methionine (M), and valine (V) and a mutation at 195, the mutation being an aliphatic hydrophobic residue selected from glycine (G), alanine (A), leucine (L), methionine (M), and valine (V) and a mutation at N96, the mutation being an aliphatic hydrophobic residue selected from glycine (G), alanine (A), leucine (L), isoleucine (1), methionine (M), and valine (V).

In some embodiments, the mutant IFNβ comprises SEQ ID NO: 38 and a mutation at K123, the mutation being an aliphatic hydrophobic residue selected from glycine (G), alanine (A), leucine (L), isoleucine (1), methionine (M), and valine (V).

In some embodiments, the mutant IFNβ comprises SEQ ID NO: 38 and a mutation at R124, the mutation being an aliphatic hydrophobic residue selected from glycine (G), alanine (A), leucine (L), isoleucine (1), methionine (M), and valine (V).

In some embodiments, the mutant IFNβ comprises SEQ ID NO: 38 and a mutation at K123, the mutation being an aliphatic hydrophobic residue selected from glycine (G), alanine (A), leucine (L), isoleucine (1), methionine (M), and valine (V) and a mutation at R124, the mutation being an aliphatic hydrophobic residue selected from glycine (G), alanine (A), leucine (L), isoleucine (1), methionine (M), and valine (V).

In some embodiments, the mutant IFNβ comprises SEQ ID NO: 38 and a mutation at L151, the mutation being an aliphatic hydrophobic residue selected from glycine (G), alanine (A), isoleucine (1), methionine (M), and valine (V).

In some embodiments, the mutant IFNβ comprises SEQ ID NO: 38 and a mutation at R152, the mutation being an aliphatic hydrophobic residue selected from glycine (G), alanine (A), leucine (L), isoleucine (1), methionine (M), and valine (V).

In some embodiments, the mutant IFNβ comprises SEQ ID NO: 38 and a mutation at L151, the mutation being an aliphatic hydrophobic residue selected from glycine (G), alanine (A), isoleucine (1), methionine (M), and valine (V) and a mutation at R152, the mutation being an aliphatic hydrophobic residue selected from glycine (G), alanine (A), leucine (L), isoleucine (1), methionine (M), and valine (V).

In some embodiments, the mutant IFNβ comprises SEQ ID NO: 38 and a mutation at V148, the mutation being an aliphatic hydrophobic residue selected from glycine (G), alanine (A), leucine (L), isoleucine (1), and methionine (M).

In some embodiments, the mutant IFNβ comprises SEQ ID NO: 38 and a mutation at V148, the mutation being an aliphatic hydrophobic residue selected from glycine (G), alanine (A), leucine (L), isoleucine (1), methionine (M), and valine (V) and a mutation at R152, the mutation being an aliphatic hydrophobic residue selected from glycine (G), alanine (A), leucine (L), isoleucine (1), methionine (M), and valine (V).

In some embodiments, the mutant IFNβ comprises SEQ ID NO: 38 and a mutation at Y155, the mutation being an aliphatic hydrophobic residue selected from glycine (G), alanine (A), leucine (L), isoleucine (1), methionine (M), and valine (V).

In embodiments, the wild type or modified signaling agent is IL-1β. In an embodiment, the wild type IL-1β has the amino acid sequence of:

(SEQ ID NO: 39)
APVRSLNCTLRDSQQKSLVMSGPYELKALHLQGQDMEQQWFSMSFVQGE
ESNDKIPVALGLKEKNLYLSCVLKDDKPTLQLESVDPKNYPKKKMEKRF
VFNKIEINNKLEFESAQFPNINYISTSQAENMPVFLGGTKGGQDITDFT
MQFVSS.

IL1 is a proinflammatory cytokine and an important immune system regulator. It is a potent activator of CD4 T cell responses, increases proportion of Th17 cells and expansion of IFNγ and IL-4 producing cells. IL-1 is also a potent regulator of CD8⁺ T cells, enhancing antigen-specific CD8⁺ T cell expansion, differentiation, migration to periphery and memory. IL-1 receptors comprise IL-1R1 and IL-1R2. Binding to and signaling through the IL-1R1 constitutes the mechanism whereby IL-1 mediates many of its biological (and pathological) activities. IL1-R2 can function as a decoy receptor, thereby reducing IL-1 availability for interaction and signaling through the IL-1R1. In some embodiments, the wild type or modified signaling agent IL-1 has reduced affinity and/or activity (e.g. agonistic activity) for IL-1R1. In some embodiments, the modified IL-1 has substantially reduced or ablated affinity and/or activity for IL-1R2. In such embodiments, there is restorable IL-1/IL-1R1 signaling and prevention of loss of therapeutic chimeric proteins or chimeric protein complexes, such as an Fc-based chimeric protein complexes at IL-R2 and therefore a reduction in dose of IL-1 that is required (e.g. relative to wild type or an chimeric protein or chimeric protein complex, such as an Fc-based chimeric protein complex bearing only an attenuation mutation for IL-R1). Such constructs find use in, for example, methods of treating cancer, including, for example, stimulating the immune system to mount an anti-cancer response.

In such embodiments, the modified signaling agent has a deletion of amino acids 52-54 which produces a modified human IL-1β with reduced binding affinity for type I IL-1R and reduced biological activity. See, for example, WO 1994/000491, the entire contents of which are hereby incorporated by reference. In some embodiments, the modified human IL-1β has one or more substitution mutations selected from A117G/P118G, R120X, L122A, T125G/L126G, R127G, Q130X, Q131G, K132A, S137G/Q138Y, L145G, H146X, L145A/L147A, Q148X, Q148G/Q150G, Q150G/D151A, M152G, F162A, F162A/Q164E, F166A, Q164E/E167K, N169G/D170G, I172A, V174A, K208E, K209X, K209A/K210A, K219X, E221X, E221 S/N224A, N224S/K225S, E244K, N245Q (where X can be any change in amino acid, e.g., a non-conservative change), which exhibit reduced binding to IL-1R, as described, for example, in WO2015/007542 and WO/2015/007536, the entire contents of which is hereby incorporated by reference (numbering based on the human IL-1β sequence, Genbank accession number NP_000567, version NP-000567.1, GI: 10835145). In some embodiments, the modified human IL-1β may have one or more mutations selected from R120A, R120G, Q130A, Q130W, H146A, H146G, H146E, H146N, H146R, Q148E, Q148G, Q148L, K209A, K209D, K219S, K219Q, E221S and E221K. In an embodiment, the modified human IL-1β comprises the mutations Q131G and Q148G. In an embodiment, the modified human IL-1β comprises the mutations Q148G and K208E. In an embodiment, the modified human IL-1β comprises the mutations R120G and Q131G. In an embodiment, the modified human IL-1β comprises the mutations R120G and H146A. In an embodiment, the modified human IL-1β comprises the mutations R120G and H146N. In an embodiment, the modified human IL-1β comprises the mutations R120G and H146R. In an embodiment, the modified human IL-1β comprises the mutations R120G and H146E. In an embodiment, the modified human IL-1β comprises the mutations R120G and H146G. In an embodiment, the modified human IL-1β comprises the mutations R120G and K208E. In an embodiment, the modified human IL-1β comprises the mutations R120G, F162A, and Q164E. Modified human IL-1β mutations are relative to SEQ ID NO: 39.

In various embodiments, one or more mutations of the signaling agent may confer improved safety upon the chimeric protein or chimeric protein complex, such as an Fc-based chimeric protein complex as compared to a wild type signaling agent. The mutations may confer various other beneficial properties, including, without limitations, reduced affinity for the signaling agent's receptor and/or reduced bioactivity for the signaling agent's receptor. In some embodiments, the one or more mutations of the signaling agent allow for attenuation of the signaling agent's activity. For example, agonistic or antagonistic activity of the signaling agent can be attenuated. Furthermore, in some embodiments, the modified signaling agent comprises one or more mutations which convert its activity from agonistic to antagonistic.

In some embodiments, the signaling agent comprises one or more mutations that confer reduced affinity or activity that is restorable by attachment to one or more targeting moiety or upon inclusion in an chimeric protein or chimeric protein complex, such as an Fc-based chimeric protein complex as disclosed herein. In other embodiments, one or more mutations of the signaling agent confer substantially reduced or ablated affinity or activity that is not substantially restorable by attachment to a targeting moiety or upon inclusion in an chimeric protein or chimeric protein complex, such as an Fc-based chimeric protein complex as disclosed herein.

In some embodiments, the targeting moiety is directed against an immune cell, which can be selected from a dendritic cell, a T cell, a B cell, a macrophage, a neutrophil, myeloid derived suppressor cell, and a NK cell. In some embodiments, the targeting moiety is directed to a hematopoietic stem cell (HSC), early progenitor cell, immature thymocyte, or steady state dendritic cell (DC). The targeting moiety can functionally modulate the antigen or receptor of interest. In some embodiments, the targeting moiety binds but does not functionally modulate the antigen or receptor of interest.

### Fc Domains

The fragment crystallizable domain (Fc domain) is the tail region of an antibody that interacts with Fc receptors located on the cell surface of cells that are involved in the immune system, e.g., B lymphocytes, dendritic cells, natural killer cells, macrophages, neutrophils, eosinophils, basophils, and mast cells. In IgG, IgA and IgD antibody isotypes, the Fc domain is composed of two identical protein fragments, derived from the second and third constant domains of the antibody's two heavy chains. In IgM and IgE antibody isotypes, the Fc domain contains three heavy chain constant domains (C_{H} domains 2-4) in each polypeptide chain.

In some embodiments, the Fc-based chimeric protein of complex the present technology includes an Fc domain.

In some embodiments, the Fc domains are from selected from IgG, IgA, IgD, IgM or IgE. In some embodiments, the Fc domains are from selected from IgG1, IgG2, IgG3, or IgG4.

In some embodiments, the Fc domains are from selected from human IgG, IgA, IgD, IgM or IgE. In some embodiments, the Fc domains are from selected from human IgG1, IgG2, IgG3, or IgG4.

In some embodiments, the Fc domains of the chimeric protein or chimeric protein complex, such as an Fc-based chimeric protein complex comprises the CH2 and CH3 regions of IgG. In some embodiments, the IgG is human IgG. In some embodiments, the human IgG is selected from IgG1, IgG2, IgG3, or IgG4.

In some embodiments, the Fc domains comprise one or more mutations. In some embodiments, the mutation(s) to the Fc domains reduces or eliminates the effector function the Fc domains. In some embodiments, the mutated Fc domain has reduced affinity or binding to a target receptor. By way of example, in some embodiments, the mutation to the Fc domains reduces or eliminates the binding of the Fc domains to FcγR. In some embodiments, the FcγR is selected from FcγRI; FcγRIIa, 131 R/R; FcγRIIa, 131 H/H, FcγRIIb; and FcγRIII. In some embodiments, the mutation to the Fc domains reduces or eliminated binding to complement proteins, such as, e.g., C1q. In some embodiments, the mutation to the Fc domains reduces or eliminated binding to both FcγR and complement proteins, such as, e.g., C1q.

In some embodiments, the Fc domains comprise the LALA mutation to reduce or eliminate the effector function of the Fc domains. By way of example, in some embodiments, the LALA mutation comprises L234A and L235A substitutions in human IgG (e.g., IgG1) (wherein the numbering is based on the commonly used numbering of the CH2 residues for human IgG1 according to EU convention (PNAS, Edelman et al., 1969; 63 (1) 78-85)). In some embodiments, the Fc domains of human IgG comprise a mutation at 46. to reduce or eliminate the effector function of the Fc domains. By way of example, in some embodiments, the mutations are selected from L234A, L234F, L235A, L235E, L235Q, K322A, K322Q, D265A, P329G, P329A, P331G, and P331S.

In some embodiments, the Fc domains comprise the FALA mutation to reduce or eliminate the effector function of the Fc domains. By way of example, in some embodiments, the FALA mutation comprises F234A and L235A substitutions in human IgG4.

In some embodiments, the Fc domains of human IgG4 comprise a mutation at one or more of F234, L235, K322, D265, and P329 to reduce or eliminate the effector function of the Fc domains. By way of example, in some embodiments, the mutations are selected from F234A, L235A, L235E, L235Q, K322A, K322Q, D265A, P329G, and P329A.

In some embodiments, the mutation(s) to the Fc domain stabilize a hinge region in the Fc domain. By way of example, in some embodiments, the Fc domain comprises a mutation at S228 of human IgG to stabilize a hinge region. In some embodiments, the mutation is S228P.

In some embodiments, the mutation(s) to the Fc domain promote chain pairing in the Fc domain. In some embodiments, chain pairing is promoted by ionic pairing (a/k/a charged pairs, ionic bond, or charged residue pair).

In some embodiments, the Fc domain comprises a mutation at one more of the following amino acid residues of IgG to promote of ionic pairing: D356, E357, L368, K370, K392, D399, and K409.

By way of example, in some embodiments, the human IgG Fc domain comprise one of the mutation combinations in Table 1 to promote of ionic pairing.

TABLE 1

| Substitution(s) on one Fc Chain | Substitution(s) on other Fc Chain |
|---|---|
| D356K D399K | K392D K409D |
| E357R L368R | K370D K409D |
| E357R L368K | K370D K409D |
| E357R D399K | K370D K409D |
| E357R | K370D |
| L368R D399K | K392D K409D |
| L368K D399K | K392D K409D |
| L368R D399K | K409D |
| L368K D399K | K409D |
| L368R | K409D |
| L368K | K409D |
| K370D K409D | E357R D399K |
| K370D K409D | E357R L368R |
| K370D K409D | E357R L368K |
| K370D K409D | E357R D399K |
| K370D K409D | E357R L368R |
| K370D K409D | E357R L368K |
| K370D | E357R |
| K370D | E357R |
| K392D K409D | D356K D399K |
| K392D K409D | L368R D399K |
| K392D K409D | L368K D399K |
| K392D K409D | D399K |
| D399K | K392D K409D |
| D399K | K409D |
| K409D | L368R |
| K409D | L368K |
| K409D | L368R D399K |
| K409D | L368K D399K |
| K409D | L368R |
| K409D | L368K |
| K409D | L368R D399K |
| K409D | L368K D399K |
| K409D | D399K |

In some embodiments, chain pairing is promoted by a knob-in-hole mutations. In some embodiments, the Fc domain comprises one or more mutations to allow for a knob-in-hole interaction in the Fc domain. In some embodiments, a first Fc chain is engineered to express the “knob” and a second Fc chain is engineered to express the complementary “hole.” By way of example, in some embodiments, human IgG Fc domain comprises the mutations of Table 2 to allow for a knob-in-hole interaction.

TABLE 2

| Substitution(s) on one Fc Chain | Substitution(s) on other Fc Chain |
|---|---|
| T366Y | Y407T |
| T366Y/F405A | T394W/Y407T |
| T366W | Y407A |
| T366W | Y407V |
| T366Y | Y407A |
| T366Y | Y407V |
| T366Y | Y407T |

In some embodiments, the Fc domains in the Fc-based chimeric protein complexes of the present technology comprise any combination of the above disclosed mutations. By way of example, in some embodiments, the Fc domain comprises mutations that promote ionic pairing and/or a knob-in-hole interaction. By way of example, in some embodiments, the Fc domain comprises mutations that have one or more of the following properties: promote ionic pairing, induce a knob-in-hole interaction, reduce or eliminate the effector function of the Fc domain, and cause Fc stabilization (e.g. at hinge).

By way of example, in some embodiments, a human IgG Fc domains comprise mutations disclosed in Table 3, which promote ionic pairing and/or promote a knob-in-hole interaction in the Fc domain.

TABLE 3

| Substitution(s) on one Fc Chain | Substitution(s) on other Fc Chain |
|---|---|
| T366W K370D | E357R Y407A |
| T366W K370D | E357R Y407V |
| T366W K409D | L368R Y407A |
| T366W K409D | L368R Y407V |
| T366W K409D | L368K Y407A |
| T366W K409D | L368K Y407V |
| T366W K409D | L368R D399K Y407A |
| T366W K409D | L368R D399K Y407V |
| T366W K409D | L368K D399K Y407A |
| T366W K409D | L368K D399K Y407V |
| T366W K409D | D399K Y407A |
| T366W K409D | D399K Y407V |
| T366W K392D K409D | D399K Y407A |
| T366W K392D K409D | D399K Y407V |
| T366W K392D K409D | D356K D399K Y407A |
| T366W K392D K409D | D356K D399K Y407V |
| T366W K370D K409D | E357R D399K Y407A |
| T366W K370D K409D | E357R D399K Y407V |
| T366W K370D K409D | E357R L368R Y407A |
| T366W K370D K409D | E357R L368R Y407V |
| T366W K370D K409D | E357R L368K Y407A |
| T366W K370D K409D | E357R L368K Y407V |
| T366W K392D K409D | L368R D399K Y407A |
| T366W K392D K409D | L368R D399K Y407V |
| T366W K392D K409D | L368K D399K Y407A |
| T366W K392D K409D | L368K D399K Y407V |
| E357R T366W | K370D Y407A |
| E357R T366W | K370D Y407V |
| T366W L368R | Y407A K409D |
| T366W L368R | Y407V K409D |
| T366W L368K | Y407A K409D |
| T366W L368K | Y407V K409D |
| T366W L368R D399K | Y407A K409D |
| T366W L368R D399K | Y407V K409D |
| T366W L368K D399K | Y407A K409D |
| T366W L368K D399K | Y407V K409D |
| T366W D399K | Y407A K409D |
| T366W D399K | Y407V K409D |
| T366W D399K | K392D Y407A K409D |
| T366W D399K | K392D Y407V K409D |
| T366W D356K D399K | K392D Y407A K409D |
| T366W D356K D399K | K392D Y407V K409D |
| E357R T366W D399K | K370D Y407A K409D |
| E357R T366W D399K | K370D Y407V K409D |
| E357R T366W L368R | K370D Y407A K409D |
| E357R T366W L368R | K370D Y407V K409D |
| E357R T366W L368K | K370D Y407A K409D |
| E357R T366W L368K | K370D Y407V K409D |
| T366W L368R D399K | K392D Y407A K409D |
| T366W L368R D399K | K392D Y407V K409D |
| T366W L368K D399K | K392D Y407A K409D |

By way of example, in some embodiments, a human IgG Fc domains comprise mutations disclosed in Table 4, which promote ionic pairing, promote a knob-in-hole interaction, or a combination thereof in the Fc domain. In embodiments, the “Chain 1” and “Chain 2” of Table 4 can be interchanged (e.g. Chain 1 can have Y407T and Chain 2 can have T366Y).

TABLE 4

| Chain 1 mutation | Chain 2 mutation | Reference | IgG |
|---|---|---|---|
| T366Y | Y407T | Ridgway et al., 1996 Protein | IgG1 |
| | | Engineering, Design and Selection, | |
| | | Volume 9, Issue 7, 1 Jul. 1996, Pages | |
| | | 617-62 | |
| T366Y/F405A | T394W/Y407T | Ridgway et al., 1996 Protein | IgG1 |
| | | Engineering, Design and Selection, | |
| | | Volume 9, Issue 7, 1 Jul. 1996, Pages | |
| | | 617-62 | |
| T366W | Y407A | Atwell et al., 1997 JMB | IgG1 |
| | | Volume 270, Issue 1, 4 Jul. 1997, | |
| | | Pages 26-35 | |
| T366W | T366S/L368V/Y407A | Atwell et al., 1997 JMB | IgG1 |
| | | Volume 270, Issue 1, 4 Jul. 1997, | |
| | | Pages 26-35 | |
| T366W | L368A/Y407A | Atwell et al., 1997 JMB | IgG1 |
| | | Volume 270, Issue 1, 4 Jul. 1997, | |
| | | Pages 26-35 | |
| T366W | T366S/L368A/Y407A | Atwell et al., 1997 JMB | IgG1 |
| | | Volume 270, Issue 1, 4 Jul. 1997, | |
| | | Pages 26-35 | |
| T366W | T366S/L368G/Y407V | Atwell et al., 1997 JMB | IgG1 |
| | | Volume 270, Issue 1, 4 Jul. 1997, | |
| | | Pages 26-35 | |
| T366W/D399C | T366S/L368A/K392C/Y407V | Merchant et al., 1998 Nature | IgG1 |
| | | Biotechnology volume 16, pages 677- | |
| | | 681 (1998) | |
| T366W/K392C | T366S/L368A/D399C/Y407V | Merchant et al., 1998 Nature | IgG1 |
| | | Biotechnology volume 16, pages 677- | |
| | | 681 (1998) | |
| S354C/T366W | Y349C/T366S/L368A/Y407V | Merchant et al., 1998 Nature | IgG1 |
| | | Biotechnology volume 16, pages 677- | |
| | | 681 (1998) | |
| Y349C/T366W | S354C/T366S/L368A/Y407V | Merchant et al., 1998 Nature | IgG1 |
| | | Biotechnology volume 16, pages 677- | |
| | | 681 (1998) | |
| E356C/T366W | Y349C/T366S/L368A/Y407V | Merchant et al., 1998 Nature | IgG1 |
| | | Biotechnology volume 16, pages 677- | |
| | | 681 (1998) | |
| Y349C/T366W | E356C/T366S/L368A/Y407V | Merchant et al., 1998 Nature | IgG1 |
| | | Biotechnology volume 16, pages 677- | |
| | | 681 (1998) | |
| E357C/T366W | Y349C/T366S/L368A/Y407V | Merchant et al., 1998 Nature | IgG1 |
| | | Biotechnology volume 16, pages 677- | |
| | | 681 (1998) | |
| Y349C/T366W | E357C/T366S/L368A/Y407V | Merchant et al., 1998 Nature | IgG1 |
| | | Biotechnology volume 16, pages 677- | |
| | | 681 (1998) | |
| D339R | K409E | Gunasekaran et al., 2010 The Journal of | IgG1 |
| | | Biological Chemistry 285, 19637-19646. | |
| D339K | K409E | Gunasekaran et al., 2010 The Journal of | IgG1 |
| | | Biological Chemistry 285, 19637-19646. | |
| D339R | K409D | Gunasekaran et al., 2010 The Journal of | IgG1 |
| | | Biological Chemistry 285, 19637-19646. | |
| D339K | K409D | Gunasekaran et al., 2010 The Journal of | IgG1 |
| | | Biological Chemistry 285, 19637-19646. | |
| D339K | K360D/K409E | Gunasekaran et al., 2010 The Journal of | IgG1 |
| | | Biological Chemistry 285, 19637-19646. | |
| D339K | K392D/K409E | Gunasekaran et al., 2010 The Journal of | IgG1 |
| | | Biological Chemistry 285, 19637-19646. | |
| D339K/E356K | K392D/K409E | Gunasekaran et al., 2010 The Journal of | IgG1 |
| | | Biological Chemistry 285, 19637-19646. | |
| D339K/E357K | K392D/K409E | Gunasekaran et al., 2010 The Journal of | IgG1 |
| | | Biological Chemistry 285, 19637-19646. | |
| D339K/E356K | K409E/K439D | Gunasekaran et al., 2010 The Journal of | IgG1 |
| | | Biological Chemistry 285, 19637-19646. | |
| D339K/E357K | K370D/K409E | Gunasekaran et al., 2010 The Journal of | IgG1 |
| | | Biological Chemistry 285, 19637-19646. | |
| D339K/E356K/E357K | K370D/K392D/K409E | Gunasekaran et al., 2010 The Journal of | IgG1 |
| | | Biological Chemistry 285, 19637-19646. | |
| S364H/F405A | Y349T/T394F | Moore et al., 2011 mAbs, 3: 6, 546-557 | IgG1 |
| S364H/T394F | Y349T/F405A | Moore et al., 2011 mAbs, 3: 6, 546-557 | IgG1 |
| D221R/P228R/K409R | D221E/P228E/L368E | Strop et al., 2012 JMB Volume 420, | IgG1 |
| | | Issue 3, 13 Jul. 2012, Pages 204-219 | |
| C223R/E225R/P228R/K409R | C223E/P228E/L368E | Strop et al., 2012 JMB Volume 420, | IgG2 |
| | | Issue 3, 13 Jul. 2012, Pages 204-219 | |
| F405L | K409R | Labrijn et al., 2013 PNAS Mar. 26, | IgG1 |
| | | 2013, 110 (13) 5145-5150 | |
| F405A/Y407V | T394W | Von Kreudenstein et al., 2013 mAbs | IgG1 |
| | | Volume 5, 2013 - Issue 5, pp. 644-654 | |
| F405A/Y407V | T366I/T394W | Von Kreudenstein et al., 2013 mAbs | IgG1 |
| | | Volume 5, 2013 - Issue 5, pp.644-654 | |
| F405A/Y407V | T366L/T394W | Von Kreudenstein et al., 2013 mAbs | IgG1 |
| | | Volume 5, 2013 - Issue 5, pp. 644-654 | |
| F405A/Y407V | T366L/K392M/T394W | Von Kreudenstein et al., 2013 mAbs | IgG1 |
| | | Volume 5, 2013 - Issue 5, pp. 644-654 | |
| L351Y/F405A/Y407V | T366L/K392M/T394W | Von Kreudenstein et al., 2013 mAbs | IgG1 |
| | | Volume 5, 2013 - Issue 5, pp. 644-654 | |
| T350V/L351Y/F405A/Y407V | T350V/T366L/K392M/T394W | Von Kreudenstein et al., 2013 mAbs | IgG1 |
| | | Volume 5, 2013 - Issue 5, pp. 644-654 | |
| T350V/L351Y/F405A/Y407V | T350V/T366L/K392L/T394W | Von Kreudenstein et al., 2013 mAbs | IgG1 |
| | | Volume 5, 2013 - Issue 5, pp. 644-654 | |
| K409W | D339V/F405T | Choi et al., 2013 PNAS Jan. 2, | IgG1 |
| | | 2013. 110 (1) 270-275 | |
| K360E | Q347R | Choi et al., 2013 PNAS Jan. 2, | IgG1 |
| | | 2013. 110 (1) 270-275 | |
| K360E/K409W | D339V/Q347R/F405T | Choi et al., 2013 PNAS Jan. 2, | IgG1 |
| | | 2013. 110 (1) 270-275 | |
| Y349C/K360E/K409W | D339V/Q347R/S354C/F405T | Choi et al., 2013 PNAS Jan. 2, | IgG1 |
| | | 2013. 110 (1) 270-275 | |
| K392A/K409D | E356K/D399K | Leaver-Fey et al., 2016 Structure | IgG1 |
| | | Volume 24, Issue 4, 5 Apr. 2016, Pages | |
| | | 641-651 | |
| T366W | T366S/L358A/Y407A | Leaver-Fey et al., 2016 Structure | IgG1 |
| | | Volume 24, Issue 4, 5 Apr. 2016, Pages | |
| | | 641-651 | |
| D339M/Y407A | T336V/K409V | Leaver-Fey et al., 2016 Structure | IgG1 |
| | | Volume 24, Issue 4, 5 Apr. 2016, Pages | |
| | | 641-651 | |
| D339M/K360D/Y407A | T336V/E345R/Q347R/K409V | Leaver-Fey et al., 2016 Structure | IgG1 |
| | | Volume 24, Issue 4, 5 Apr. 2016, Pages | |
| | | 641-651 | |
| Y349S/T366V/K370Y/K409V | E357D/S364Q/Y407A | Leaver-Fey et al., 2016 Structure | IgG1 |
| | | Volume 24, Issue 4, 5 Apr. 2016, Pages | |
| | | 641-651 | |
| Y349S/T366M/K370Y/K409V | E356G/E357D/S364Q/Y407A | Leaver-Fey et al., 2016 Structure | IgG1 |
| | | Volume 24, Issue 4, 5 Apr. 2016, Pages | |
| | | 641-651 | |
| Y349S/T366M/K370Y/K409V | E357D/S364R/Y407A | Leaver-Fey et al., 2016 Structure | IgG1 |
| | | Volume 24, Issue 4, 5 Apr. 2016, Pages | |
| | | 641-651 | |

And any combination as described in Tables 1-3 of US20150284475A1

By way of example, in some embodiments, a human IgG Fc domains comprise mutations disclosed in Table 5, which reduce or eliminate FcγR and/or complement binding in the Fc domain. In embodiments, the table 5 mutations are in both chains.

TABLE 5

| Chain 1 mutation | Reference | IgG |
|---|---|---|
| L234A/L235A | Alegre et al., 1994 | IgG1 |
| | Transplantation 57: 1537- | |
| | 1543 | |
| F234A/L235A | Alegre et al., 1994 | IgG4 |
| | Transplantation 57: 1537- | |
| | 1543 | |
| L235E | Morgan et al., 1995 | IgG1 |
| | Immunology. 1995 October; | |
| | 86(2): 319-324. | |
| L235E | Morgan et al., 1995 | IgG4 |
| | Immunology. 1995 October; | |
| | 86(2): 319-324. | |
| L235A | Morgan et al., 1995 | IgG1 |
| | Immunology. 1995 October; | |
| | 86(2): 319-324. | |
| G237A | Morgan et al., 1995 | IgG1 |
| | Immunology. 1995 October; | |
| | 86(2): 319-324. | |
| N297H | Tao and Morrison, | IgG1 |
| | J. Immunol. 1989; 143: | |
| | 2595-2601 | |
| N297Q | Tao and Morrison, | IgG1 |
| | J. Immunol. 1989; 143: | |
| | 2595-2601 | |
| N297K | Tao and Morrison, | lgG3 |
| | J. Immunol. 1989; 143: | |
| | 2595-2601 | |
| N297Q | Tao and Morrison, | lgG3 |
| | J. Immunol. 1989; 143: | |
| | 2595-2601 | |
| D265A | Idusogie et al., 2000 J | IgG1 |
| | Immunol Apr. 15, 2000, 164 | |
| | (8) 4178-4184 | |
| D270A, V, K | Idusogie et al., 2000 J | IgG1 |
| | Immunol Apr. 15, 2000, 164 | |
| | (8) 4178-4184 | |
| K322A, L, M, D, E | Idusogie et al., 2000 J | IgG1 |
| | Immunol Apr. 15, 2000, 164 | |
| | (8) 4178-4184 | |
| P329A, X | Idusogie et al., 2000 J | IgG1 |
| | Immunol Apr. 15, 2000, 164 | |
| | (8) 4178-4184 | |
| P331A, S, G, X | Idusogie et al., 2000 J | IgG1 |
| | Immunol Apr. 15, 2000, 164 | |
| | (8) 4178-4184 | |
| D265A | Idusogie et al., 2000 J | IgG1 |
| | Immunol Apr. 15, 2000, 164 | |
| | (8) 4178-4184 | |
| L234A | Hezareh et al., 2001 J. Virol. | IgG1 |
| | December 2001 vol. 75 no. | |
| | 24 12161-12168 | |
| L234A/L235A | Hezareh et al., 2001 J. Virol. | IgG1 |
| | December 2001 vol. 75 no. | |
| | 24 12161-12168 | |
| L234F/L235E/P331S | Oganesyan et al., 2008 Acta | IgG1 |
| | Cryst. (2008). D64, 700-704 | |
| H268Q/V309L/A330S/P331S | An et al., 2009 mAbs | IgG1 |
| | Volume 1, 2009 - Issue 6, | |
| | pp. 572-579 | |
| G236R/L328R | Moore et al., 2011 mAbs | IgG1 |
| | Volume 3, 2011 - Issue 6, | |
| | pp. 546-557 | |
| N297G | Couch et al., 2013 Sci. | IgG1 |
| | Transl. Med., 5 (2013) | |
| | 183ra57, 1-12 | |
| N297G/D265A | Couch et al., 2013 Sci. | IgG1 |
| | Transl. Med., 5 (2013) | |
| | 183ra57, 1-12 | |
| V234A/G237A/P328S/H268A/ | Vafa et al., 2014 Methods | IgG2 |
| V309L/A330S/P331S | Volume 65, Issue 1, 1 | |
| | Jan. 2014, Pages 114-126 | |
| L234A/L235A/P329G | Lo et al., 2016 The Journal | IgG1 |
| | of Biological Chemistry | |
| | 292, 3900-3908 | |
| N297D | Schlothauer et al., 2016 | IgG1 |
| | Protein Engineering, Design | |
| | and Selection, Volume 29, | |
| | Issue 10, 1 Oct. 2016, | |
| | Pages 457-466 | |
| S228P/L235E | Schlothauer et al., 2016 | IgG4 |
| | Protein Engineering, Design | |
| | and Selection, Volume 29, | |
| | Issue 10, 1 Oct. 2016, | |
| | Pages 457-466 | |
| S228P/L235E/P329G | Schlothauer et al., 2016 | IgG4 |
| | Protein Engineering, Design | |
| | and Selection, Volume 29, | |
| | Issue 10, 1 Oct. 2016, | |
| | Pages 457-466 | |
| L234F/L235A/K322Q | Borrok et al., 2017 J Pharm | IgG1 |
| | Sci April 2017 Volume 106, | |
| | Issue 4, Pages 1008-1017 | |
| L234F/L235Q/P331G | Borrok et al., 2017 J Pharm | IgG1 |
| | Sci April 2017 Volume 106, | |
| | Issue 4, Pages 1008-1017 | |
| L234F/L235Q/K322Q | Borrok et al., 2017 J Pharm | IgG1 |
| | Sci April 2017 Volume 106, | |
| | Issue 4, Pages 1008-1017 | |
| L234A/L235A/G237A/P328S/ | Tam et al., 2017 Open Access | IgG1 |
| H268A/A330S/P331S | Antibodies 2017, 6(3), 12; | |
| | doi: 10.3390/antib6030012 | |
| S228P/F234A/L235A | Tam et al., 2017 Open Access | IgG4 |
| | Antibodies 2017, 6(3), 12; | |
| | doi: 10.3390/antib6030012 | |
| S228P/F234A/L235A/G237A/ | Tam et al., 2017 Open Access | IgG4 |
| P238S | Antibodies 2017, 6(3), 12; | |
| | doi: 10.3390/antib6030012 | |
| S228P/F234A/L235A/G236B/ | Tam et al., 2017 Open Access | IgG4 |
| G237A/P238S | Antibodies 2017, 6(3), 12; | |
| | doi: 10.3390/antib6030012 | |

In some embodiments, the Fc domains in the Fc-based chimeric protein complexes of the present technology are homodimeric, i.e., the Fc region in the chimeric protein complex comprises two identical protein fragments.

In some embodiments, the Fc domains in the Fc-based chimeric protein complexes of the present technology are heterodimeric, i.e., the Fc domain comprises two non-identical protein fragments.

In some embodiments, heterodimeric Fc domains are engineered using ionic pairing and/or knob-in-hole mutations described herein. In some embodiments, the heterodimeric Fc-based chimeric protein complexes have a trans orientation. In a trans orientation, the targeting moiety and signaling agent are, in embodiments, not found on the same polypeptide chain in the present Fc-based chimeric protein complexes.

In some embodiments, heterodimeric Fc domains are engineered using ionic pairing and/or knob-in-hole mutations described herein. In some embodiments, the heterodimeric Fc-based chimeric protein complexes have a trans orientation.

In a trans orientation, the targeting moiety and signaling agent are, in embodiments, not found on the same polypeptide chain in the present Fc-based chimeric protein complexes. In a trans orientation, the targeting moiety and signaling agent are, in embodiments, found on separate polypeptide chains in the Fc-based chimeric protein complexes. In a cis orientation, the targeting moiety and signaling agent are, in embodiments, found on the same polypeptide chain in the Fc-based chimeric protein complexes.

In some embodiments, where more than one targeting moiety is present in the heterodimeric protein complexes described herein, one targeting moiety may be in trans orientation (relative to the signaling agent), whereas another targeting moiety may be in cis orientation (relative to the signaling agent). In some embodiments, the signaling agent and target moiety are on the same ends/sides (N-terminal or C-terminal ends) of an Fc domain. In some embodiments, the signaling agent and targeting moiety are on different sides/ends of an Fc domain (N-terminal and C-terminal ends).

In some embodiments, where more than one targeting moiety is present in the heterodimeric protein complexes described herein, the targeting moieties may be found on the same Fc chain or on two different Fc chains in the heterodimeric protein complex (in the latter case the targeting moieties would be in trans relative to each other, as they are on different Fc chains). In some embodiments, where more than one targeting moiety is present on the same Fc chain, the targeting moieties may be on the same or different sides/ends of an Fc chain (N-terminal or/and C-terminal ends).

In some embodiments, where more than one signaling agent is present in the heterodimeric protein complexes described herein, the signaling agents may be found on the same Fc chain or on two different Fc chains in the heterodimeric protein complex (in the latter case the signaling agents would be in trans relative to each other, as they are on different Fc chains). In some embodiments, where more than one signaling agent is present on the same Fc chain, the signaling agents may be on the same or different sides/ends of an Fc chain (N-terminal or/and C-terminal ends).

In some embodiments, where more than one signaling agent is present in the heterodimeric protein complexes described herein, one signaling agent may be in trans orientation (as relates to the targeting moiety), whereas another signaling agent may be in cis orientation (as relates to the targeting moiety).

In some embodiments, for a “split” targeting moiety, as described for the FLT3L-ECD, and variants thereof such as the FLT3L-ECD with a L27D mutation, parts of a targeting moiety may be present on each of the Fc chains of a homodimeric or heterodimeric protein complex, and formation of a functional targeting moiety is generated as part of formation of the chimeric protein complex, as exemplified with FLT3L-ECD monomers that have dimerized to form a functional FLT3 targeting moiety for delivery of a signaling agent activity to a FLT3-positive target cell. In some embodiments, the Fc domains includes or starts with the core hinge region of wild-type human IgG1, which contains the sequence Cys-Pro-Pro-Cys (SEQ ID NO: 42). In some embodiments, the Fc domains also include the upper hinge, or parts thereof (e.g., DKTHTCPPC (SEQ ID NO: 43); see WO 2009053368), EPKSCDKTHTCPPC (SEQ ID NO: 44), or EPKSSDKTHTCPPC (SEQ ID NO: 45); see Lo et al., Protein Engineering vol. 11 no. 6 pp. 495-500, 1998)).

### Fc-Based Chimeric Protein Complexes

The Fc-based chimeric protein complexes of the present technology comprise at least one Fc domain disclosed herein, at least one signaling agent (SA) disclosed herein, and at least one targeting moiety (TM) disclosed herein.

It is understood that, the present Fc-based chimeric protein complexes may encompass a complex of two fusion proteins, each comprising an Fc domain.

In some embodiments, the Fc-based chimeric protein complex is heterodimeric. In some embodiments, the heterodimeric Fc-based chimeric protein complex has a trans orientation. In some embodiments, the heterodimeric Fc-based chimeric protein complex has a cis orientation. In some embodiments, the heterodimeric Fc-based chimeric protein complex does not comprise the signaling agent and targeting moiety on a single polypeptide.

In some embodiments, the Fc-based chimeric protein has an improved in vivo half-life relative to a chimeric protein lacking an Fc or a chimeric protein which is not a heterodimeric complex. In some embodiments, the Fc-based chimeric protein has an improved solubility, stability and other pharmacological properties relative to a chimeric protein lacking an Fc or a chimeric protein which is not a heterodimeric complex.

Heterodimeric Fc-based chimeric protein complexes are composed of two different polypeptides. In embodiments described herein, the targeting domain is on a different polypeptide than the signaling agent and accordingly, proteins that contain only one targeting domain copy, and also only one signaling agent copy can be made (this provides a configuration in which potential interference with desired properties can be controlled). Further, in embodiments, one targeting domain (e.g. VHH) only can avoid cross-linking of the antigen on the cell surface (which could elicit undesired effects in some cases) Further, in embodiments, one signaling agent may alleviate molecular “crowding” and potential interference with avidity mediated restoration of effector function in dependence of the targeting domain. Further, in embodiments, heterodimeric Fc-based chimeric protein complexes can have two targeting moieties, and these can be placed on the two different polypeptides. For instance, in embodiments, the C-terminus of both targeting moieties (e.g. VHHs) can be masked to avoid potential autoantibodies or pre-existing antibodies (e.g. VHH autoantibodies or pre-existing antibodies). Further, in embodiments, heterodimeric Fc-based chimeric protein complexes, e.g. with the targeting domain on a different polypeptide than the signaling agent (e.g. wild type signaling agent), may favor “cross-linking” of two cell types (e.g. a tumor cell and an immune cell). Further, in embodiments, heterodimeric Fc-based chimeric protein complexes can have two signaling agents, each on different polypeptides to allow more complex effector responses.

Further, in embodiments, heterodimeric Fc-based chimeric protein complexes, e.g. with the targeting domain on a different polypeptide than the signaling agent, combinatorial diversity of targeting moiety and signaling agent is provided in a practical manner. For instance, in embodiments, polypeptides with any of the targeting moieties described herein can be combined “off the shelf” with polypeptides with any of the signaling agents described herein to allow rapid generation of various combinations of targeting moieties and signaling agents in single Fc-based chimeric protein complexes.

In some embodiments, the Fc-based chimeric protein complex comprises one or more linkers. In some embodiments, the Fc-based chimeric protein complex includes a linker that connects the Fc domain, signaling agent(s) and targeting moiety(ies). In some embodiments, the Fc-based chimeric protein complex includes a linker that connects each signaling agent and targeting moiety (or, if more than one targeting moiety, a signaling agent to one of the targeting moieties). In some embodiments, the Fc-based chimeric protein complex includes a linker that connects each signaling agent to the Fc domain. In some embodiments, the Fc-based chimeric protein complex includes a linker that connects each targeting moiety to the Fc domain. In some embodiments, the Fc-based chimeric protein complex includes a linker that connects a targeting moiety to another targeting moiety. In some embodiments, the Fc-based chimeric protein complex includes a linker that connects a signaling agent to another signaling agent.

In some embodiments, an Fc-based chimeric protein complex comprises two or more targeting moieties. In such embodiments, the targeting moieties can be the same targeting moiety or they can be different targeting moieties.

In some embodiments, an Fc-based chimeric protein complex comprises two or more signaling agents. In such embodiments, the signaling agents can be the same targeting moiety or they can be different targeting moieties.

By way of example, in some embodiments, the Fc-based chimeric protein complex comprise an Fc domain, at least two signaling agents (SA), and at least two targeting moieties (TM), wherein the Fc domain, signaling agents, and targeting moieties are selected from any of the Fc domains, signaling agents, and targeting moieties disclosed herein. In some embodiments, the Fc domain is homodimeric.

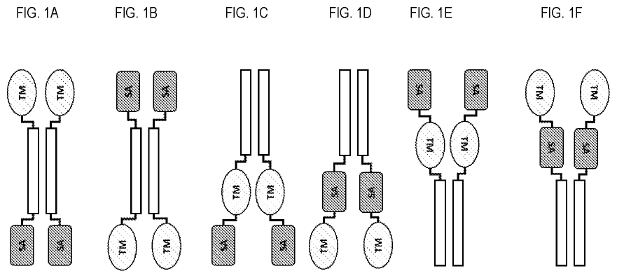

In various embodiments, the Fc-based chimeric protein complex takes the form of any of the schematics of FIGS. 1A-F.

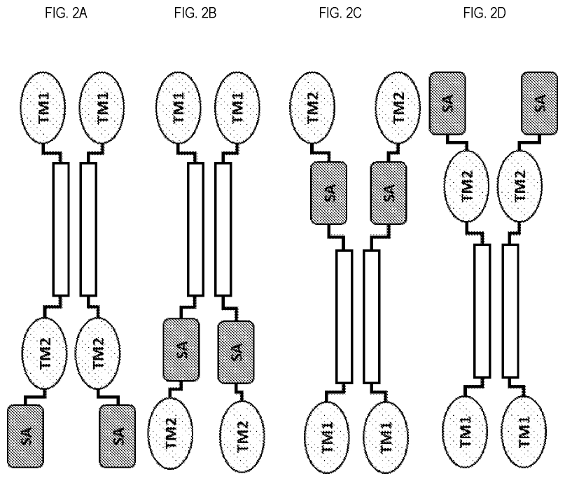

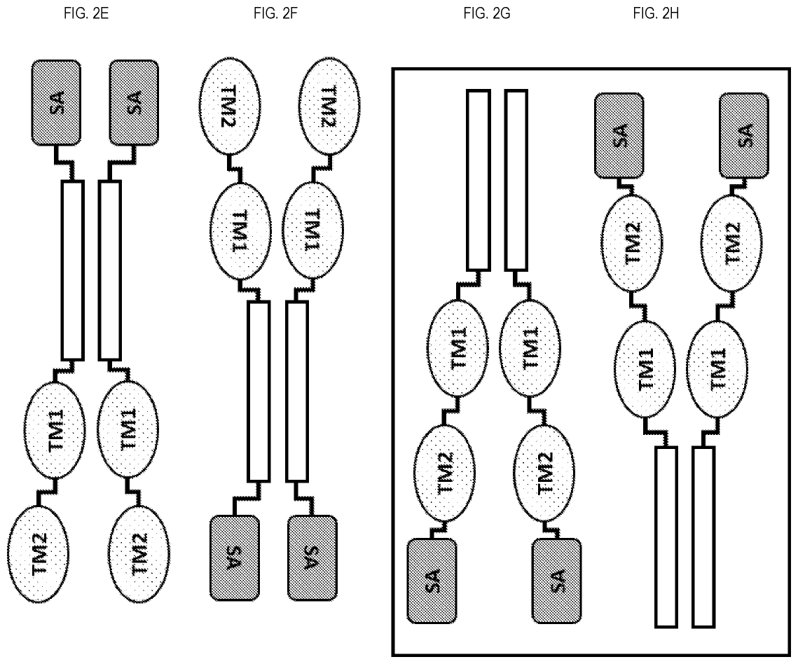

In various embodiments, the Fc-based chimeric protein complex takes the form of any of the schematics of FIGS. 2A-H.

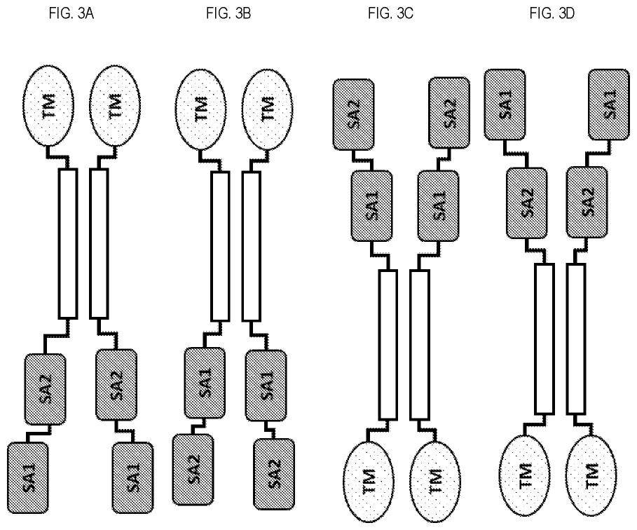

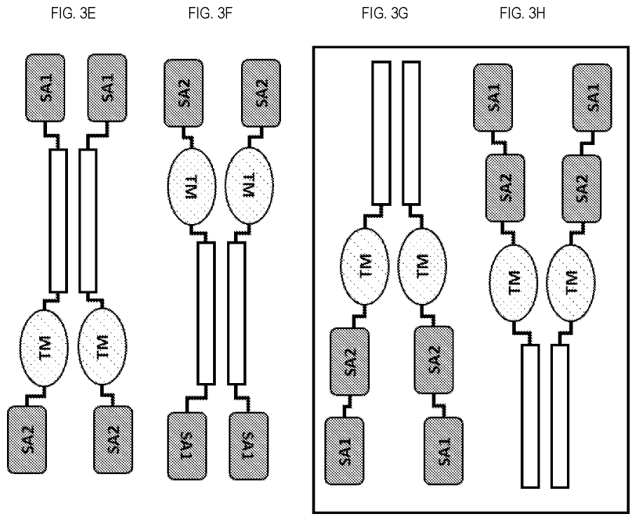

In various embodiments, the Fc-based chimeric protein complex takes the form of any of the schematics of FIGS. 3A-H.

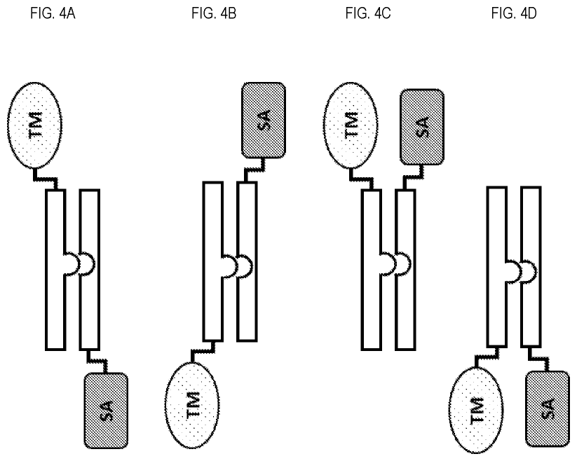

In various embodiments, the Fc-based chimeric protein complex takes the form of any of the schematics of FIGS. 4A-D.

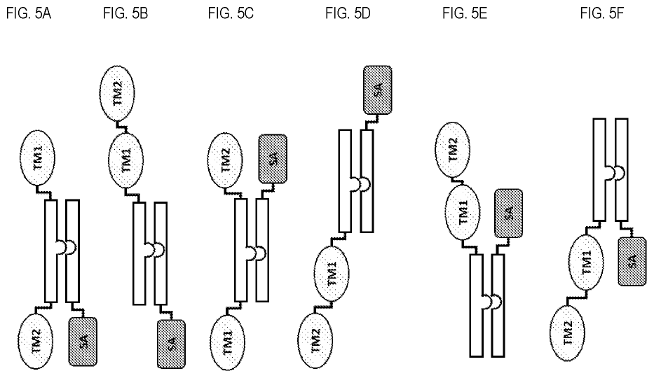

In various embodiments, the Fc-based chimeric protein complex takes the form of any of the schematics of FIGS. 5A-F.

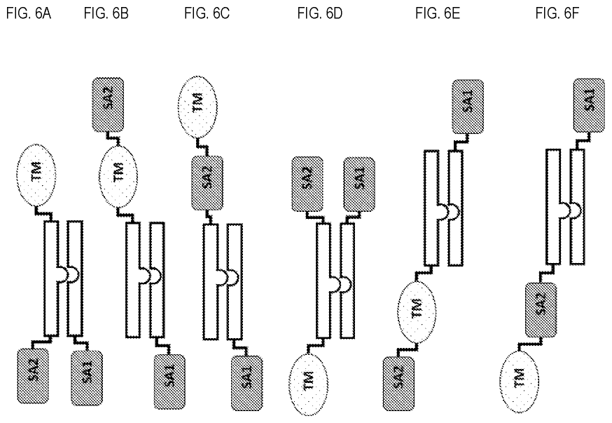

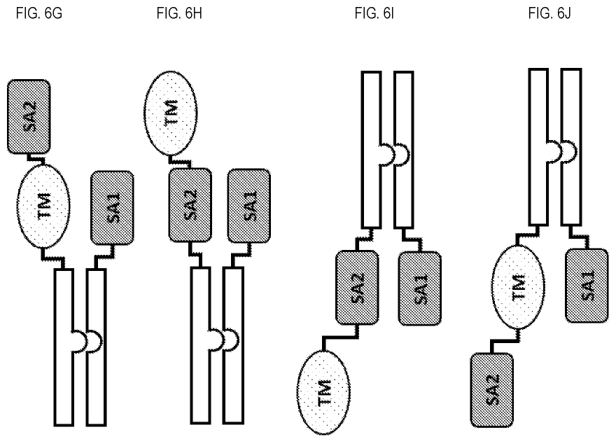

In various embodiments, the Fc-based chimeric protein complex takes the form of any of the schematics of FIGS. 6A-J.

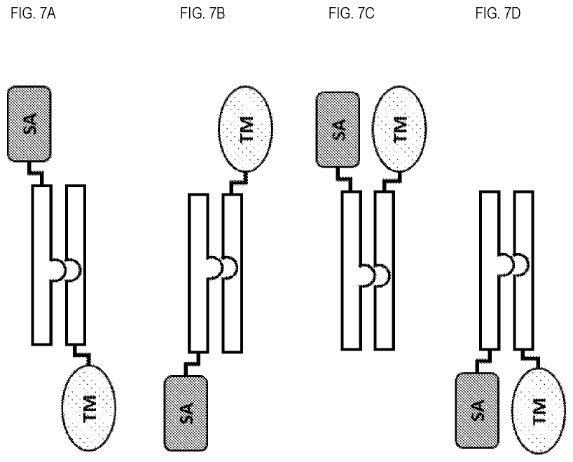

In various embodiments, the Fc-based chimeric protein complex takes the form of any of the schematics of FIGS. 7A-D.

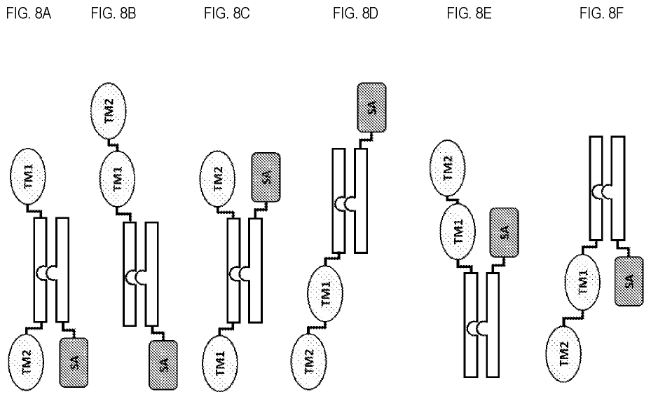

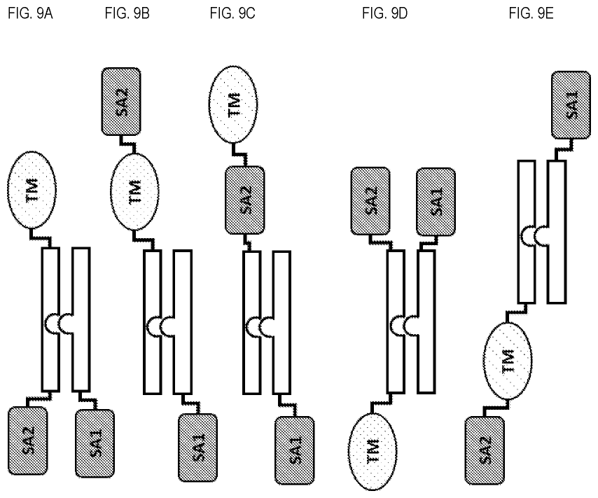

In various embodiments, the Fc-based chimeric protein complex takes the form of any of the schematics of FIGS. 8A-F.

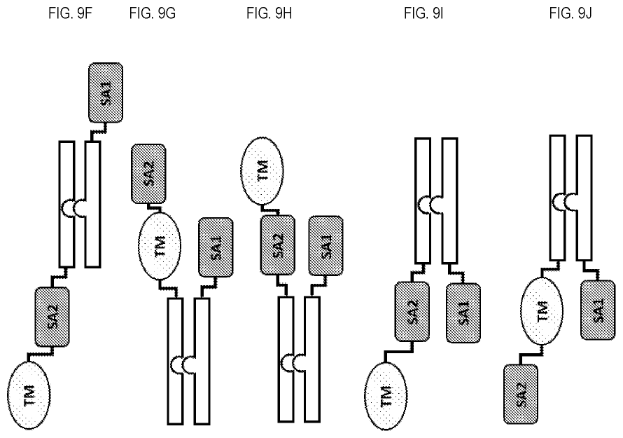

In various embodiments, the Fc-based chimeric protein complex takes the form of any of the schematics of FIGS. 9A-J.

In various embodiments, the Fc-based chimeric protein complex takes the form of any of the schematics of FIGS. 10A-F.

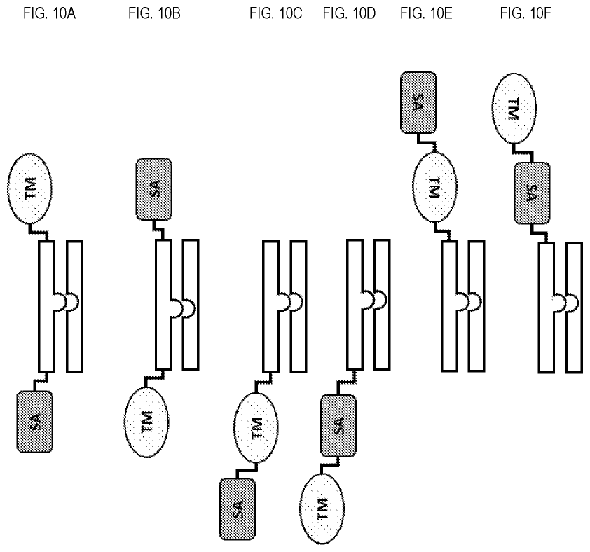

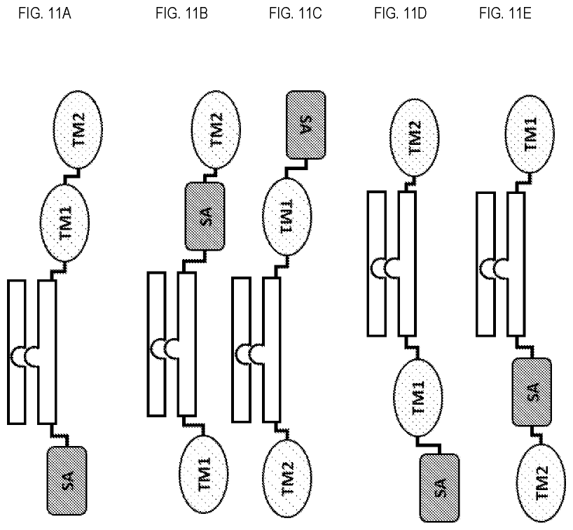

In various embodiments, the Fc-based chimeric protein complex takes the form of any of the schematics of FIGS. 11A-L.

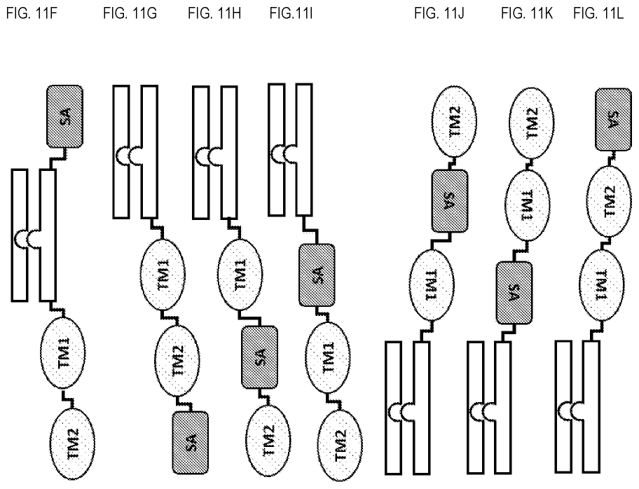

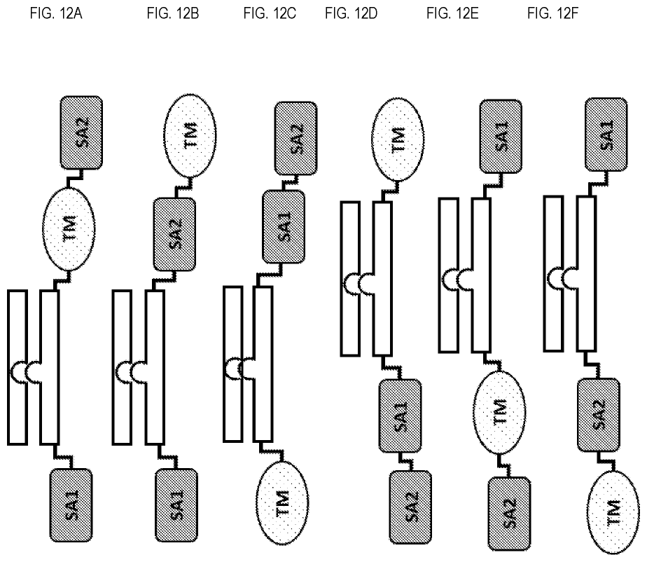

In various embodiments, the Fc-based chimeric protein complex takes the form of any of the schematics of FIGS. 12A-L.

In various embodiments, the Fc-based chimeric protein complex takes the form of any of the schematics of FIGS. 13A-F.

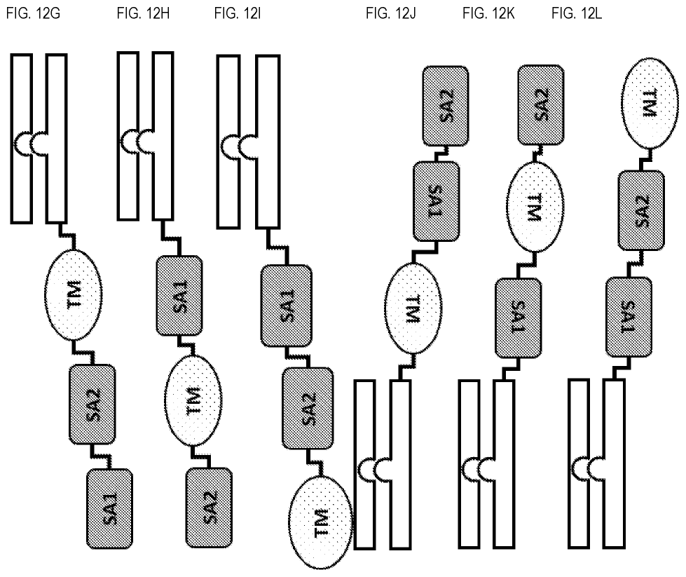

In various embodiments, the Fc-based chimeric protein complex takes the form of any of the schematics of FIGS. 14A-L.

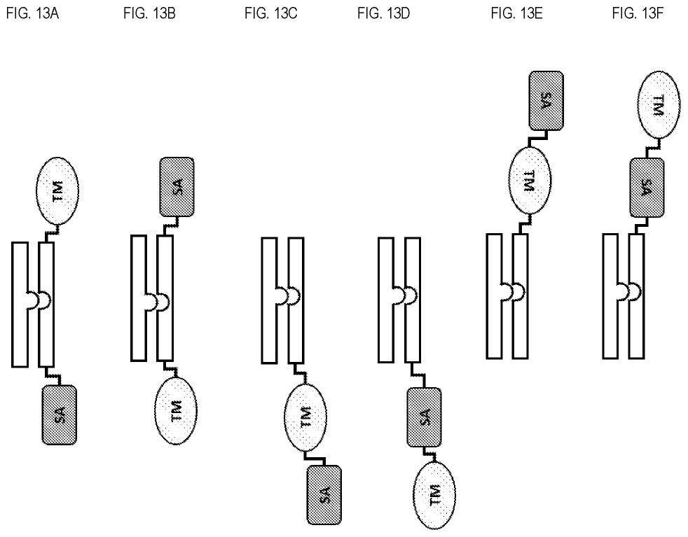

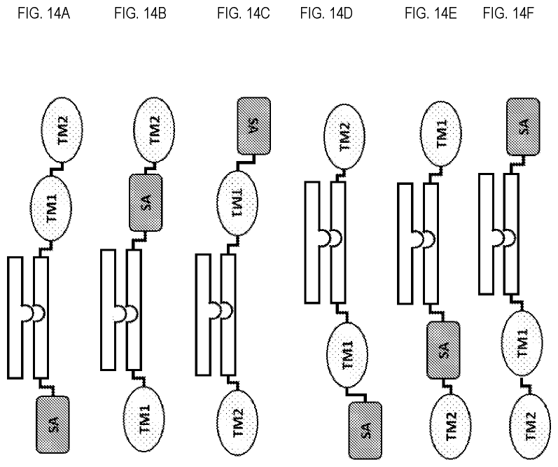

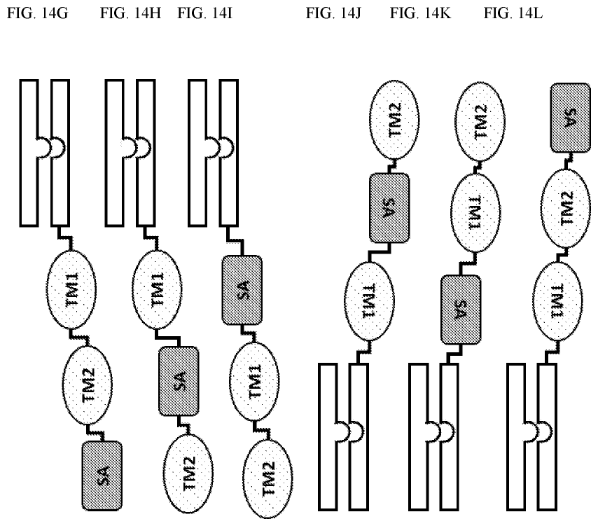

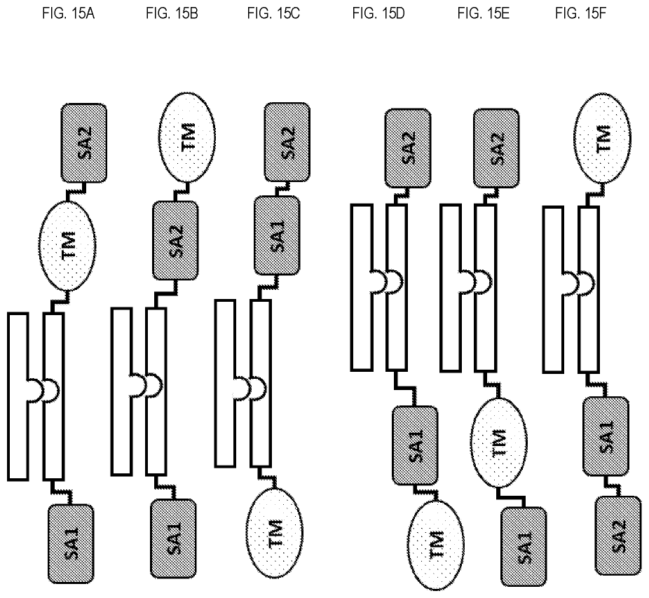

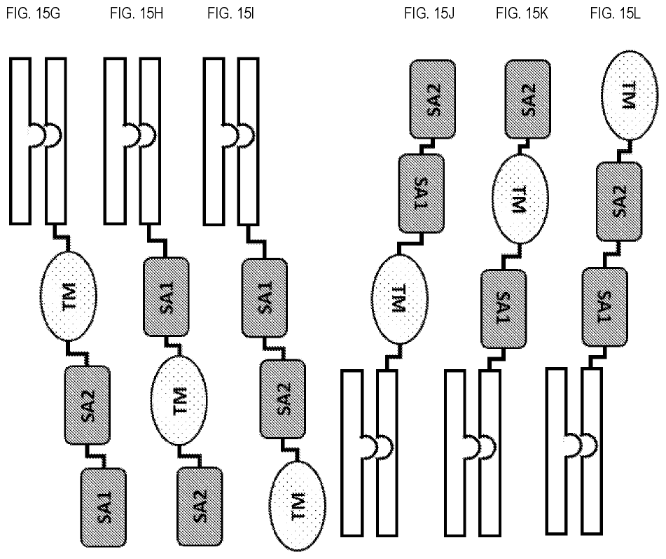

In various embodiments, the Fc-based chimeric protein complex takes the form of any of the schematics of FIGS. 15A-L.

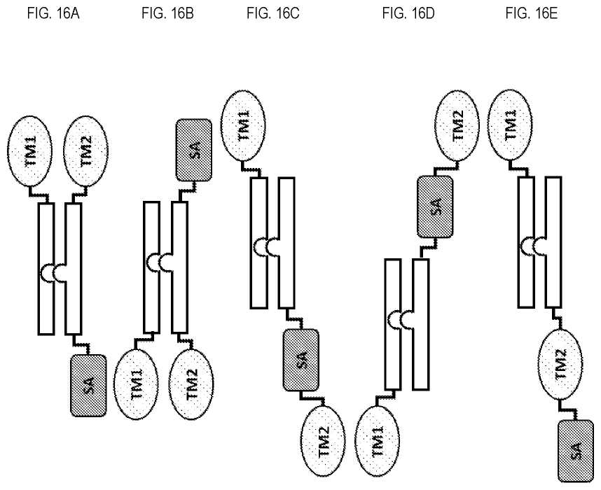

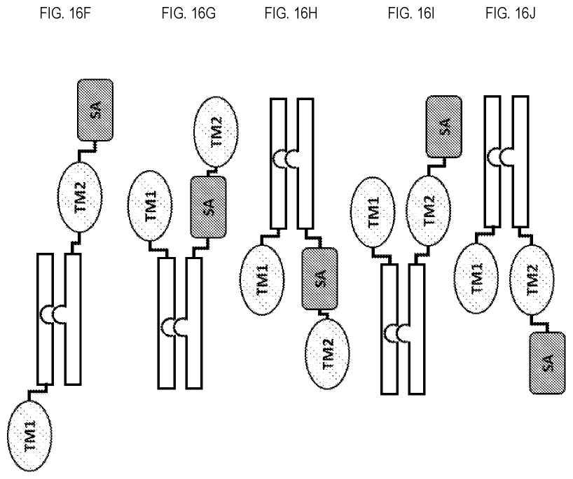

In various embodiments, the Fc-based chimeric protein complex takes the form of any of the schematics of FIGS. 16A-J.

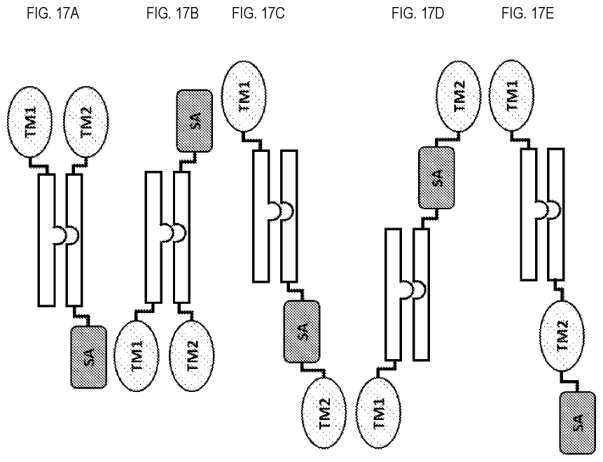

In various embodiments, the Fc-based chimeric protein complex takes the form of any of the schematics of FIGS. 17A-J.

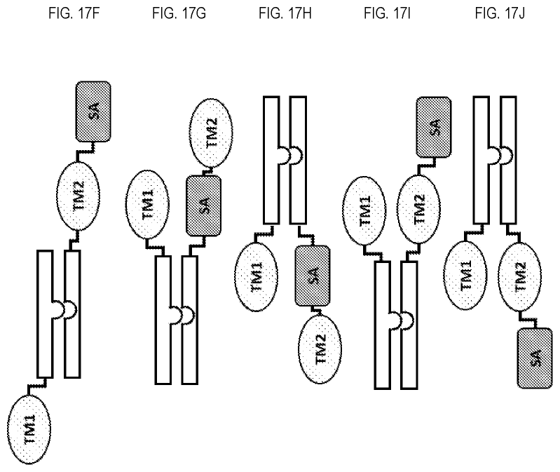

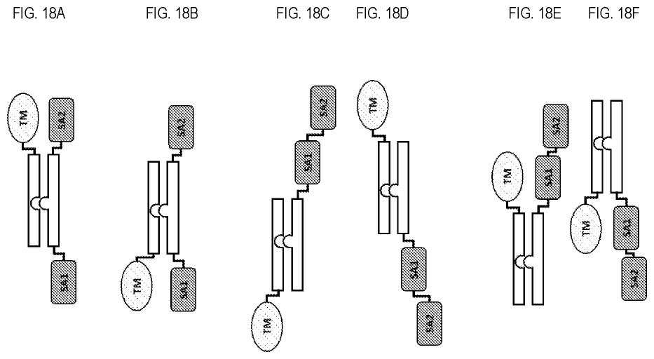

In various embodiments, the Fc-based chimeric protein complex takes the form of any of the schematics of FIGS. 18A-F.

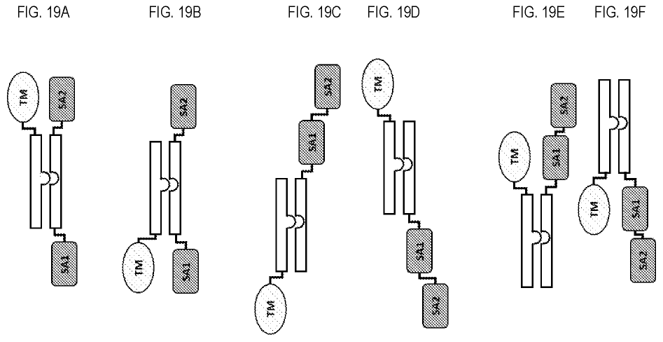

In various embodiments, the Fc-based chimeric protein complex takes the form of any of the schematics of FIGS. 19A-F.

In various embodiments, the Fc-based chimeric protein complex takes the form of any of the schematics of FIGS. 21A-F.

In various embodiments, the Fc-based chimeric protein complex takes the form of any of the schematics of FIGS. 22A-F.

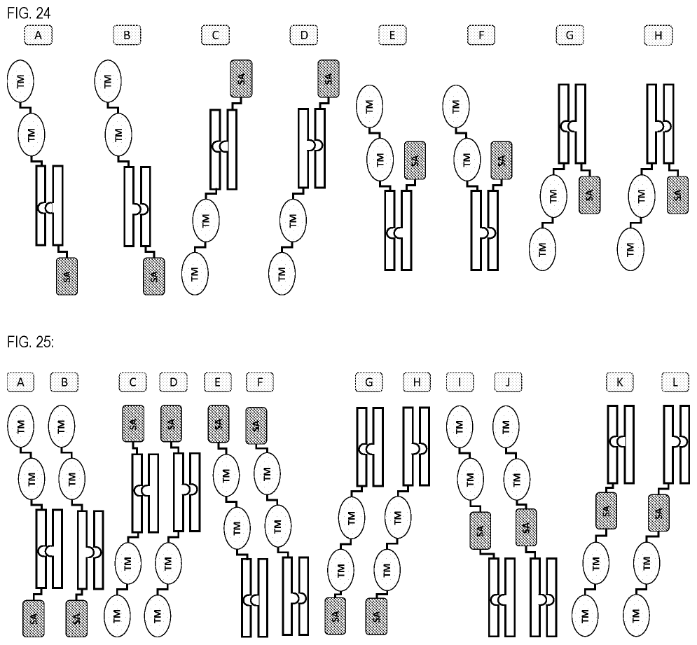

In various embodiments, the Fc-based chimeric protein complex takes the form of any of the schematics of FIGS. 24A-H.

In various embodiments, the Fc-based chimeric protein complex takes the form of any of the schematics of FIGS. 25A-L.

In some embodiments, the signaling agents are linked to the targeting moieties and the targeting moieties are linked to the Fc domain on the same terminus (see FIGS. 1A-F). In some embodiments, the Fc domain is homodimeric.

In some embodiments, the signaling agents and targeting moieties are linked to the Fc domain, wherein the targeting moieties and signaling agents are linked on the same terminus (see FIGS. 1A-F). In some embodiments, the Fc domain is homodimeric.

In some embodiments, the targeting moieties are linked to signaling agents and the signaling agents are linked to the Fc domain on the same terminus (see FIGS. 1A-F). In some embodiments, the Fc domain is homodimeric.

In some embodiments, the homodimeric Fc-based chimeric protein complex has two or more targeting moieties.

In some embodiments, there are four targeting moieties and two signaling agents, the targeting moieties are linked to the Fc domain and the signaling agents are linked to targeting moieties on the same terminus (see FIGS. 2A-H). In some embodiments, the Fc domain is homodimeric. In some embodiments, where there are four targeting moieties and two signaling agents, two targeting moieties are linked to the Fc domain and two targeting moieties are linked to the signaling agents, which are linked to the Fc domain on the same terminus (see FIGS. 2A-H). In some embodiments, the Fc domain is homodimeric. In some embodiments, where there are four targeting moieties and two signaling agents, two targeting moieties are linked to each other and one of the targeting moieties of from each pair is linked to the Fc domain on the same terminus and the signaling agents are linked to the Fc domain on the same terminus (see FIGS. 2A-H). In some embodiments, the Fc domain is homodimeric. In some embodiments, where there are four targeting moieties and two signaling agents, two targeting moieties are linked to each other, wherein one of the targeting moieties of from each pair is linked to a signaling agent and the other targeting moiety of the pair is linked the Fc domain, wherein the targeting moieties linked to the Fc domain are linked on the same terminus (see FIGS. 2A-H). In some embodiments, the Fc domain is homodimeric.

In some embodiments, the homodimeric Fc-based chimeric protein complex has two or more signaling agents. In some embodiments, where there are four signaling agents and two targeting moieties, two signaling agents are linked to each other and one of the signaling agents of from pair is linked to the Fc domain on the same terminus and the targeting moieties are linked to the Fc domain on the same terminus (see FIGS. 3A-H). In some embodiments, the Fc domain is homodimeric. In some embodiments, where there are four signaling agents and two targeting moieties, two signaling agents are linked to the Fc domain one the same terminus and two of the signaling agents are each linked to a targeting moiety, wherein the targeting moieties are linked to the Fc domain at the same terminus (see FIGS. 3A-H). In some embodiments, the Fc domain is homodimeric. In some embodiments, where there are four signaling agents and two targeting moieties, two signaling agents are linked to each other and one of the signaling agents of from pair is linked to a targeting moiety and the targeting moieties are linked to the Fc domain on the same terminus (see FIGS. 3A-H). In some embodiments, the Fc domain is homodimeric.

By way of example, in some embodiments, the Fc-based chimeric protein complex comprise an Fc domain, wherein the Fc domain comprises ionic pairing mutation(s) and/or knob-in-hole mutation(s), at least one signaling agent, and at least one targeting moiety, wherein the ionic pairing motif and/or a knob-in-hole motif, signaling agent, and targeting moiety are selected from any of the ionic pairing motif and/or a knob-in-hole motif, signaling agents, and targeting moieties disclosed herein. In some embodiments, the Fc domain is heterodimeric. In some embodiments, the Fc domain comprises a mutation that reduces or eliminates its effector function.

In some embodiments, the signaling agent is linked to the targeting moiety, which is linked to the Fc domain (see FIGS. 10A-F and 13A-F). In some embodiments, the targeting moiety is linked to the signaling agent, which is linked to the Fc domain (see FIGS. 10A-F and 13A-F). In some embodiments, the Fc domain is heterodimeric. In some embodiments, the Fc domain comprises a mutation that reduces or eliminates its effector function.

In some embodiments, the signaling agent and targeting moiety are linked to the Fc domain (see FIGS. 4A-D, 7A-D, 10A-F, and 13A-F). In some embodiments, the targeting moiety and the signaling agent are linked to different Fc chains on the same terminus (see FIGS. 4A-D and 7A-D). In some embodiments, the targeting moiety and the signaling agent are linked to different Fc chains on different termini (see FIGS. 4A-D and 7A-D). In some embodiments, the targeting moiety and the signaling agent are linked to the same Fc chain (see FIGS. 10A-F and 13A-F). In some embodiments, the Fc domain is heterodimeric. In some embodiments, the Fc domain comprises a mutation that reduces or eliminates its effector function.

In some embodiments, where there are one signaling agent and two targeting moieties, the signaling agent is linked to the Fc domain and two targeting moieties can be: 1) linked to each other with one of the targeting moieties linked to the Fc domain; or 2) each linked to the Fc domain (see FIGS. 5A-F, 8A-F, 11A-L, 14A-L, 16A-J, and 17A-J). In some embodiments, the targeting moieties are linked on one Fc chain and the signaling agent is on the other Fc chain (see FIGS. 5A-F and 8A-F). In some embodiments, the paired targeting moieties and the signaling agent are linked to the same Fc chain (see FIGS. 11A-L and 14A-L). In some embodiments, a targeting moiety is linked to the Fc domain and the other targeting moiety is linked to the signaling agent, and the paired targeting moiety is linked to the Fc domain (see FIGS. 11A-L, 14A-L, 16A-J, and 17A-J). In some embodiments, the unpaired targeting moiety and paired targeting moiety are linked to the same Fc chain (see FIGS. 11A-L and 14A-L). In some embodiments, the unpaired targeting moiety and paired targeting moiety are linked to different Fc chains (see FIGS. 16A-J and 17A-J). In some embodiments, the unpaired targeting moiety and paired targeting moiety are linked on the same terminus (see FIGS. 16A-J and 17A-J). In some embodiments, the Fc domain is heterodimeric.

In some embodiments, the Fc domain comprises a mutation that reduces or eliminates its effector function.

In some embodiments, where there are one signaling agent and two targeting moieties, a targeting moiety is linked to the signaling agent, which is linked to the Fc domain, and the unpaired targeting moiety is linked the Fc domain (see FIGS. 11A-L, 14A-L, 16A-J, and 17A-J). In some embodiments, the paired signaling agent and unpaired targeting moiety are linked to the same Fc chain (see FIGS. 11A-L and 14A-L). In some embodiments, the paired signaling agent and unpaired targeting moiety are linked to different Fc chains (see FIGS. 16A-J and 17A-J). In some embodiments, the paired signaling agent and unpaired targeting moiety are linked on the same terminus (see FIGS. 16A-J and 17A-J). In some embodiments, the Fc domain is heterodimeric. In some embodiments, the Fc domain comprises a mutation that reduces or eliminates its effector function.

In some embodiments, where there are one signaling agent and two targeting moieties, the targeting moieties are linked together and the signaling agent is linked to one of the paired targeting moieties, wherein the targeting moiety not linked to the signaling agent is linked to the Fc domain (see FIGS. 11A-L and 14A-L). In some embodiments, the Fc domain is heterodimeric. In some embodiments, the Fc domain comprises a mutation that reduces or eliminates its effector function.

In some embodiments, where there are one signaling agent and two targeting moieties, the targeting moieties are linked together and the signaling agent is linked to one of the paired targeting moieties, wherein the signaling agent is linked to the Fc domain (see FIGS. 11A-L and 14A-L). In some embodiments, the Fc domain is heterodimeric. In some embodiments, the Fc domain comprises a mutation that reduces or eliminates its effector function.

In some embodiments, where there are one signaling agent and two targeting moieties, the targeting moieties are both linked to the signaling agent, wherein one of the targeting moieties is linked to the Fc domain (see FIGS. 11A-L and 14A-L). In some embodiments, the Fc domain is heterodimeric. In some embodiments, the Fc domain comprises a mutation that reduces or eliminates its effector function.

In some embodiments, where there are one signaling agent and two targeting moieties, the targeting moieties and the signaling agent are linked to the Fc domain (see FIGS. 16A-J and 17A-J). In some embodiments, the targeting moieties are linked on the terminus (see FIGS. 16A-J and 17A-J). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked to the Fc domain on the same terminus and the targeting moiety is linked to the Fc domain (see FIGS. 6A-J and 9A-J). In some embodiments, the signaling agents are linked to the Fc domain on the same Fc chain and the targeting moiety is linked on the other Fc chain (see FIGS. 18A-F and 19A-F). In some embodiments, the Fc domain is heterodimeric. In some embodiments, the Fc domain comprises a mutation that reduces or eliminates its effector function.

In some embodiments, where there are two signaling agents and one targeting moiety, a signaling agent is linked to the targeting moiety, which is linked to the Fc domain and the other signaling agent is linked to the Fc domain (see FIGS. 6A-J, 9A-J, 12A-L, and 15A-L). In some embodiments, the targeting moiety and the unpaired signaling agent are linked to different Fc chains (see FIGS. 6A-J and 9A-J). In some embodiments, the targeting moiety and the unpaired signaling agent are linked to different Fc chains on the same terminus (see FIGS. 6A-J and 9A-J). In some embodiments, the targeting moiety and the unpaired signaling agent are linked to different Fc chains on different termini (see FIGS. 6A-J and 9A-J). In some embodiments, the targeting moiety and the unpaired signaling agent are linked to the same Fc chains (see FIGS. 12A-L and 15A-L). In some embodiments, the Fc domain is heterodimeric. In some embodiments, the Fc domain comprises a mutation that reduces or eliminates its effector function.

In some embodiments, where there are two signaling agents and one targeting moiety, the targeting moiety is linked to a signaling agent, which is linked to the Fc domain and the other signaling agent is linked to the Fc domain (see FIGS. 6A-J and 9A-J). In some embodiments, the paired signaling agent and the unpaired signaling agent are linked to different Fc chains (see FIGS. 6A-J and 9A-J). In some embodiments, the paired signaling agent and the unpaired signaling agent are linked to different Fc chains on the same terminus (see FIGS. 6A-J and 9A-J). In some embodiments, the paired signaling agent and the unpaired signaling agent are linked to different Fc chains on different termini (see FIGS. 6A-J and 9A-J). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and the targeting moiety is linked to one of the paired signaling agents, wherein the targeting moiety is linked to the Fc domain (see FIGS. 12A-L and 15A-L). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and one of the signaling agents is linked to the Fc domain and the targeting moiety is linked to the Fc domain (see FIGS. 12A-L, 15A-L, 18A-F, and 19A-F). In some embodiments, the paired signaling agents and targeting moiety are linked to the same Fc chain (see FIGS. 12A-L and 15A-L). In some embodiments, the paired signaling agents and targeting moiety are linked to different Fc chains (see FIGS. 18A-F and 19A-F). In some embodiments, the paired signaling agents and targeting moiety are linked to different Fc chains on the same terminus (see FIGS. 18A-F and 19A-F). In some embodiments, the Fc domain is heterodimeric. In some embodiments, the Fc domain comprises a mutation that reduces or eliminates its effector function.

In some embodiments, where there are two signaling agents and one targeting moiety, the signaling agents are both linked to the targeting moiety, wherein one of the signaling agents is linked to the Fc domain (see FIGS. 12A-L and 15A-L). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and one of the signaling agents is linked to the targeting moiety and the other signaling agent is linked to the Fc domain (see FIGS. 12A-L and 15A-L).

In some embodiments, where there are two signaling agents and one targeting moiety, each signaling agent is linked to the Fc domain and the targeting moiety is linked to one of the signaling agents (see FIGS. 12A-L and 15A-L). In some embodiments, the signaling agents are linked to the same Fc chain (see FIGS. 12A-L and 15A-L).

In some embodiments, a targeting moiety or signaling agent is linked to the Fc domain, comprising one or both of C_{H}2 and C_{H}3 domains, and optionally a hinge region. For example, vectors encoding the targeting moiety, signaling agent, or combination thereof, linked as a single nucleotide sequence to an Fc domain can be used to prepare such polypeptides.

In some embodiments, for a “split” targeting moiety, as described for the FLT3L-ECD, and variants thereof such as the FLT3L-ECD with a L27D mutation, parts of a targeting moiety may be present on each of the Fc chains of a homodimeric or heterodimeric protein complex, and formation of a functional targeting moiety is generated as part of formation of the chimeric protein complex, as exemplified with FLT3L-ECD monomers that have dimerized to form a functional FLT3 targeting moiety for delivery of a signaling agent activity to a FLT3-positive target cell.

### Non-Fc-Based Chimeric Protein Complexes

In various embodiments, the chimeric protein complexes of the present invention include (a) one or more targeting moieties comprising a FMS-like tyrosine kinase 3 ligand (FLT3L) domain, e.g., without limitation, Flt3L's extracellular domain as described herein, e.g., without limitation, single chain or split chain and optionally with the L27D mutation and (b) a wild type or modified signaling agent as described herein; wherein (a) and (b) are connected with a domain that causes complexation (e.g. a complexation domain). In some embodiments, the chimeric protein complexes of the present invention further include one or more proteins or peptides that interact with each other (e.g. a complexation domain), e.g., using electrostatic interactions, hydrogen bonding, and/or the hydrophobic effect. In some embodiments, the chimeric protein complexes are homomers (e.g., that include two or more chimeric proteins as described herein). In some embodiments, the chimeric protein complexes are heteromers (e.g., that include one chimeric protein comprising a wild type or modified signaling agent and one or more targeting moieties connected with one or more linkers and another protein). A variety of protein interaction domains (e.g. a complexation domains) have been employed to generate protein complexes and can be used for the purposes of making chimeric protein complexes of the present invention. In some embodiments, the chimeric protein complexes can be made by using leucine zippers, Jun and Fos family of proteins, helix-turn-helix self dimerizing peptides, tri- and tetrameric subdomains of collagen and p53 (see, e.g. methods of making protein complexes as described in U.S. Pat. No. 8,507,222, which is hereby incorporated by reference in its entirety). Other methods to make heteromeric complexes include charge based heterodimers as e.g. described by Chang et al. (PNAS 1984; 91:11408-11412) or heterodimerizing leucine zippers as described e.g. by Deng et al. (Chemistry & Biology 2008; 15:908-919) or designed heterodimers as described by Chen et al. (Nature 2019; 565:106-111). In various embodiments, these chimeric protein complexes, are not Fc-based. In some embodiments, the variety of protein interaction domains can be used in place of Fc-domains described herein (in the context of Fc-based chimeric protein complexes) to form protein complexes.

### Non-Fc Single Chain Flt3L Constructs

In some aspects, the present invention is related to a chimeric protein comprising (i) one or more targeting moieties comprising a FMS-like tyrosine kinase 3 ligand (FLT3L) domain, wherein the FLT3L domain is a single chain dimer and; (ii) one or more flexible linkers connecting elements (i) and (iii); and (iii) a signaling agent or a modified form thereof.

In embodiments, the chimeric protein or protein complex has a targeting moiety that comprises the extracellular domain of FLT3L, or a portion or variant thereof. In some embodiments, the targeting moiety comprises an amino acid sequence having at least 90% identity with any one of SEQ ID NOs: 2-5. In some embodiments, the targeting moiety comprises an amino acid sequence having at least 95% identity with any one of SEQ ID NOs: 2-5. In some embodiments the chimeric protein or protein complex comprises two copies of the FLT3L-ECD, or variants thereof, on the same polypeptide (i.e., a single chain dimeric FLT3L construct). In some embodiments the FLT3L-ECD, or a portion or variant thereof, contains a mutation that reduces intermolecular FLT3L-ECD homodimerization (i.e., dimerization of FLT3L domains on separate FLT3L-ECD containing molecules that would not otherwise readily dimerize by other mechanisms), and favoring intramolecular FLT3L-ECD dimerization (i.e., dimerization of two copies of FLT3L-ECDs contained within the same, single polypeptide, i.e., a single chain dimeric FLT3L construct). In some embodiments, the mutation in the FLT3L-ECD, or variant thereof, is L27D (with reference to any one of SEQ ID NOs: 2-4 or L24D with regard to SEQ ID NO: 5). In some embodiments, mutation L27D (with reference to any one of SEQ ID NOs: 2-4 or L24D with regard to SEQ ID NO: 5), or functionally similar mutations in the FLT3-ECD, or variants thereof, favor the formation of more homogenous forms of chimeric proteins and protein complexes, avoiding formation of undesired and higher molecular weight complexes and/or aggregates that would be substantially detrimental to scale up production of the FLT3-targeted construct and in vivo safety of such constructs (e.g., risk of immunoreactivity, loss of activity etc.).

In some embodiments, a single copy of FLT3L-ECD, having a L27D mutation or functionally equivalent mutation in the ECD, may be present in a polypeptide that itself can dimerize with the same or different polypeptide that also has a single copy of FLT3L-ECD having a L27D mutation, or functionally equivalent mutation in the ECD. Upon formation of a protein complex, dimerization of the FLT3L-ECD-L27D domain is induced, thereby generating a functional FLT3-targeting moiety. This type of chimeric protein complex is considered to harbor a “split FLT3L-ECD”, as functional targeting moiety from split FLT3-ECDs (i.e., on different polypeptide molecules) is created upon assembly of the chimeric protein complex. A signaling agent may be attached or fused to either one of the polypeptides that form the chimeric protein complex.

Several types of signaling agents can be incorporated into chimeric proteins with single chain dimeric FLT3L, or portions and variants thereof, and chimeric protein complexes formed with the “split FLT3L-ECD” approach. In embodiments, the chimeric protein has a targeting moiety that comprises the extracellular domain of FLT3L, or a portion thereof. In some embodiments, the targeting moiety comprises an amino acid sequence having at least 90% identity with any one of SEQ ID NOs: 2-5. In some embodiments, the targeting moiety comprises an amino acid sequence having at least 95% identity with any one of SEQ ID NOs: 2-5. In some embodiments, the signaling agent is wild type human IFNα2, IFNα1, IFNβ, or IL-1β. In some embodiments, the signaling agent comprises an amino acid sequence having at least 95% identity with any one of SEQ ID NO: 6, 7, 38, 39, or 74. In some embodiments, the signaling agent comprises an amino acid sequence of any one of SEQ ID NO: 6, 7, 38, 39, or 74.

In some embodiments, the signaling agent is modified to comprise one or more mutations. In some embodiments, the one or more mutations confer improved safety as compared to a wild type signaling agent, or reduced affinity for the signaling agent's receptor, or reduced bioactivity for the signaling agent's receptor, or allow for attenuation of the signaling agent's activity. In some embodiments, the one or more mutations confer reduced affinity or activity that is restorable by attachment to one or more targeting moiety.

In some embodiments, the chimeric protein is such that: the signaling agent is a mutant human IFNα2 comprising an amino acid sequence having at least 95% identity with SEQ ID NO: 6 or 7 and wherein the mutant human IFNα2 has one or more mutations that confer improved safety as compared to a wild type IFNα2 having an amino acid sequence of SEQ ID NO: 6 or 7, optionally wherein the human IFNα2 has one or more mutations at positions 144 to 154 with respect to SEQ ID NO: 6 or 7, optionally wherein the human IFNα2 has: one or more mutations at positions L15, A19, R22, R23, L26, F27, L30, L30, K31, D32, R33, H34, D35, Q40, H57, E58, Q61, F64, N65, T69, L80, Y85, Y89, D114, L117, R120, R125, K133, K134, R144, A145, M148, R149, S152, L153, and N156 with respect to SEQ ID NO: 6 or 7, optionally wherein the mutant human IFNα2 has one or more mutations at position R33, T106, R144, A145, M148, R149, and L153 with respect to SEQ ID NO: 6 or 7, optionally wherein the mutation is one or more of L15A, A19W, R22A, R23A, L26A, F27A, L30A, L30V, K31A, D32A, R33K, R33A, R33Q, H34A, D35A, Q40A, H57Y, E58N, Q61S, F64A, N65A, T69A, L80A, Y85A, Y89A, D114R, L117A, R120A, R125A, K133A, K134A, R144A, A145G, A145M, M148A, R149A, S152A, L153A, and N156A with respect to SEQ ID NO: 6 or 7, optionally wherein the mutant human IFNα2 has one or more mutations selected from R33A, T106X₃, R120E, R144X₁ A145X₂, M148A, R149A, and L153A with respect to amino acid sequence of SEQ ID NO: 6 or 7, wherein X₁ is selected from A, S, T, Y, L, and I, wherein X₂ is selected from G, H, Y, K, and D, and wherein X₃ is selected from A and E.

In some embodiments, the signaling agent is a wild type interferon α1 or a modified interferon α1. In some embodiments, the present invention provides a chimeric protein or Fc-based chimeric protein complex that includes a wild type IFNα1. In various embodiments, the wild-type IFNα1 comprises the amino acid sequence of SEQ ID NO: 74.

In various embodiments, the chimeric protein or Fc-based chimeric protein complex of the invention comprises a modified version of IFNα1, i.e., a IFNα1 variant including a IFNα1 mutant, as a signaling agent. In various embodiments, the IFNα1 variant encompasses mutants, functional derivatives, analogs, precursors, isoforms, splice variants, or fragments of the interferon.

In some embodiments, the IFNα1 interferon is modified to have a mutation atone or more amino acids at positions L15, A19, R23, S25, L30, D32, R33, H34, Q40, C86, D115, L118, K121, R126, E133, K134, K135, R145, A146, M149, R150, S153, L154, and N157 with reference to SEQ ID NO: 74. The mutations can optionally be a hydrophobic mutation and can be, e.g., selected from alanine, valine, leucine, and isoleucine. In some embodiments, the IFNα1 interferon is modified to have a one or more mutations selected from L15A, A19W, R23A, S25A, L30A, L30V, D32A, R33K, R33A, R33Q, H34A, Q40A, C86S, C86A, D115R, L118A, K121A, K121E, R126A, R126E, E133A, K134A, K135A, R145A, R145D, R145E, R145G, R145H, R145I, R145K, R145L, R145N, R145Q, R145S, R145T, R145V, R145Y, A146D, A146E, A146G, A146H, A146I, A146K, A146L, A146M, A146N, A146Q, A146R, A146S, A146T, A146V, A146Y, M149A, M149V, R150A, S153A, L154A, and N157A with reference to SEQ ID NO: 1042. In some embodiments, the IFNα1 mutant comprises one or more multiple mutations selected from L30A/H58Y/E59N_Q62S, R33A/H58Y/E59N/Q62S, M149A/H58Y/E59N/Q62S, L154A/H58Y/E59N/Q62S, R145A/H58Y/E59N/Q62S, D115A/R121A, L118A/R121A, L118A/R121A/K122A, R121A/K122A, and R121E/K122E with reference to SEQ ID NO: 74.

In some embodiments, the IFN-α1 is a variant that comprises one or more mutations which reduce undesired disulphide pairings wherein the one or more mutations are, e.g., at amino acid positions C1, C29, C86, C99, or C139 with reference to SEQ ID NO: 74. In some embodiments, the mutation at position C86 can be, e.g., C86S or C86A or C86Y. These C86 mutants of IFN-α1 are called reduced cysteine-based aggregation mutants. In some embodiment, the IFNα1 variant includes mutations at positions C1, C86 and C99 with reference to SEQ ID NO: 74.

In some embodiments the chimeric protein is such that: the signaling agent is a mutant human IFNβ comprising an amino acid sequence having at least 95% identity with SEQ ID NO: 38 and wherein the mutant human IFNβ has one or more mutations that confer improved safety as compared to a wild type IFNβ having an amino acid sequence of SEQ ID NO: 38, optionally wherein the mutation is one or more of W22G, R27G, L32A, L32G, R35A, R35G, V148G, L151G, R152A, R152G with respect to amino acid sequence of SEQ ID NO: 38.

In some embodiments the chimeric protein is such that: the signaling agent is a mutant human IL-1R comprising an amino acid sequence having at least 95% identity with SEQ ID NO: 39 and wherein the mutant human IL-1β has one or more mutations that confer improved safety as compared to a wild type IL-1β having an amino acid sequence of SEQ ID NO: 39, optionally wherein the mutation is one or more of A117G/P118G, R120G, R120A, L122A, T125G/L126G, R127G, Q130A, Q130W, Q131G, K132A, S137G/Q138Y, L145G, H146A, H146G, H146E, H146N, H146R, L145A/L147A, Q148E, Q148G, Q148L, Q148G/Q150G, Q150G/D151A, M152G, F162A, F162A/Q164E, F166A, Q164E/E167K, N169G/D170G, I172A, V174A, K208E, K209A, K209D, K209A/K210A, K219S, K219Q, E221S, E221K, E221S/N224A, N224S/K225S, E244K and N245Q with respect to amino acid sequence of SEQ ID NO: 39.

In some embodiments, the chimeric protein comprises a flexible linker, wherein the flexible linker is substantially comprised of glycine and serine residues, optionally wherein the flexible linker comprises (Gly₄Ser)ₙ, where n is from about 1 to about 8, optionally wherein the flexible linker comprises one or more of SEQ ID NO: 10-SEQ ID NO: 17.

In some embodiments, the present invention is related to a recombinant nucleic acid composition encoding one or more chimeric proteins described herein. In some embodiments, the present invention is related to a host cell comprising the recombinant nucleic acid.

In some embodiments, the chimeric protein is suitable for use in a patient having one or more of: cancer, infections, immune disorders, autoimmune and/or neurodegenerative disease, cardiovascular diseases, wound, ischemia-related diseases, and/or metabolic diseases. Some aspects of the present invention are related to a method for treating or preventing cancer, infections, immune disorders, autoimmune and/or neurodegenerative disease, cardiovascular diseases, wound, ischemia-related diseases, and/or metabolic diseases comprising administering an effective amount of the chimeric protein to a patient in need thereof.

### Single Chain Fc-Based Flt3L Constructs

In some aspects, the present invention is related to an Fc-based chimeric protein complex comprising: (i) one or more targeting moieties comprising a FMS-like tyrosine kinase 3 ligand (FLT3L) domain, wherein the FLT3L domain is a single chain dimer and (ii) an Fc domain, the Fc domain optionally having one or more mutations that reduces or eliminates one or more effector functions of the Fc domain, promotes Fc chain pairing in the Fc domain, and/or stabilizes a hinge region in the Fc domain.

In some embodiments, the Fc-based chimeric protein complex is such that the single chain dimeric FLT3L is attached to one Fc chain of the Fc domain. In embodiments, the Fc-based chimeric protein complex is such that the single chain dimeric FLT3L comprises the extracellular domain of FLT3L, or a portion thereof. In some embodiments, the Fc-based chimeric protein complex is such that the single chain dimeric FLT3L comprises an amino acid sequence having at least 90% identity with any one of SEQ ID NOs: 2-5. In some embodiments, the single chain dimeric FLT3L comprises an amino acid sequence having at least 95% identity with any one of SEQ ID NOs: 2-5.

In some embodiments the Fc-based chimeric protein complex comprises two copies of the FLT3L-ECD (FLT3L extracellular domain), or variants thereof, attached to one Fc chain of the Fc domain (i.e., a single chain dimeric FLT3L-ECD construct). In some embodiments the FLT3L-ECD, or a portion or variant thereof, contains a mutation that reduces intermolecular FLT3L-ECD homodimerization (i.e., dimerization of FLT3L domains present on separate FLT3L-ECD containing molecules), and favoring intramolecular FLT3L-ECD dimerization (i.e., dimerization of two copies of FLT3L-ECDs contained within the same, single polypeptide, i.e., a single chain dimeric FLT3L construct). In some embodiments, the mutation in the FLT3L-ECD, or variant thereof, is L27D (with reference to any one of SEQ ID NOs: 2-4 or L24D with regard to SEQ ID NO: 5). In some embodiments, mutation L27D (with reference to any one of SEQ ID NOs: 2-4 or L24D with regard to SEQ ID NO: 5), or functionally similar mutations in the FLT3-ECD, or variants thereof, favor the formation of more homogenous forms of chimeric proteins and protein complexes, avoiding formation of undesired and higher molecular weight complexes and/or aggregates that would be substantially detrimental to scale up production of the FLT3-targeted construct and in vivo safety of such constructs (e.g., risk of immunoreactivity, loss of activity etc.).

In some aspects, the present invention is related to a recombinant nucleic acid composition encoding one or more chimeric proteins described herein. In some embodiments, the present invention is related to a host cell that includes the recombinant nucleic acid.

In some embodiments, the Fc-based chimeric protein complex is suitable for use in a patient having one or more of: cancer, infections, immune disorders, autoimmune and/or neurodegenerative disease, cardiovascular diseases, wound, ischemia-related diseases, and/or metabolic diseases. In embodiments, the present invention is related to a method for treating or preventing cancer, infections, immune disorders, autoimmune and/or neurodegenerative disease, cardiovascular diseases, wound, ischemia-related diseases, and/or metabolic diseases comprising administering an effective amount of the Fc-based chimeric protein complex to a patient in need thereof.

### Methods of Use

In various embodiments, the chimeric protein or chimeric protein complex, such as an Fc-based chimeric protein complex, among other features, directly or indirectly recruits one or more immune cells to a disease cell, e.g. via a targeting moiety. Thus, in some embodiments, the targeting moiety directly or indirectly recruits immune cells to tumor cells or to the tumor microenvironment. In this way, the targeting moiety may increase a number of dendritic cells. In some embodiments, the targeting moiety enhances tumor antigen presentation, optionally by dendritic cells.

In various embodiments, the chimeric protein or chimeric protein complex, such as an Fc-based chimeric protein complex is suitable for use in a patient having one or more of cancer, infections, immune disorders, autoimmune and/or neurodegenerative disease, cardiovascular diseases, wound, ischemia-related diseases, and/or metabolic diseases. In some aspects, a method for treating or preventing a cancer is provided, that comprises administering an effective amount of the chimeric protein or chimeric protein complex, such as an Fc-based chimeric protein complex in accordance with various embodiments of the present disclosure to a patient in need thereof.

In various embodiments, the cancer is selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome. In certain embodiments, the cancer is acute myeloid leukemia (AML).

Furthermore, in some aspects, the present invention includes a method for treating or preventing an autoimmune and/or neurodegenerative disease, which comprises administering an effective amount of the chimeric protein or chimeric protein complex, such as an Fc-based chimeric protein complex in accordance with various embodiments of the present disclosure to a patient in need thereof. The autoimmune and/or neurodegenerative disease can be selected from multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, myasthenia gravis, Reiter's syndrome, and Grave's disease.

In some embodiments, the present chimeric protein or chimeric protein complex, such as an Fc-based chimeric protein complex optionally comprises one or more linkers. In some embodiments, the present chimeric protein or chimeric protein complex, such as an Fc-based chimeric protein complex comprises a linker connecting one or more of the Fc domain, targeting moiety and the signaling agent (e.g., IFNα2, IFNα1, IFNβ, or IL-1β or a variant thereof). In some embodiments, the present chimeric protein or chimeric protein complex, such as an Fc-based chimeric protein complex comprises a linker within the signaling agent (e.g., IFNα2, IFNα1, IFNβ, or IL-1β or a variant thereof). In some embodiments, the linker may be utilized to link various functional groups, residues, or moieties as described herein to the chimeric protein or chimeric protein complex, such as an Fc-based chimeric protein complex. In some embodiments, the linker is a single amino acid or a plurality of amino acids that does not affect or reduce the stability, orientation, binding, neutralization, and/or clearance characteristics of the binding regions and the binding protein. In various embodiments, the linker is selected from a peptide, a protein, a sugar, or a nucleic acid.

In some embodiments, the chimeric protein or chimeric protein complex, such as an Fc-based chimeric protein complex comprises one or more additional signaling agents, e.g., without limitation, an interferon, an interleukin, as described herein, that may be wild type or modified. In various embodiments, the chimeric protein or chimeric protein complex, such as an Fc-based chimeric protein complex of the invention has a modified signaling agent and provides improved safety compared to an unmodified, wild type. For clarity, the present invention includes, in embodiments, chimeric proteins or chimeric protein complexes, such as an chimeric proteins or chimeric protein complexes, such as an Fc-based chimeric protein complexes having one, or two, or three signaling agents.

In various embodiments, the chimeric protein or chimeric protein complex, such as an Fc-based chimeric protein complex comprises one or more targeting moieties (e.g., without limitation various antibody formats, inclusive of single-domain antibodies, inclusive of VHHs) which specifically bind to a target (e.g., antigen, receptor) of interest. In various embodiments, the targeting moieties specifically bind to a target (e.g., antigen, receptor) of interest, including those found on one or more immune cells, which can include, without limitation, T cells, cytotoxic T lymphocytes, T helper cells, natural killer (NK) cells, natural killer T (NKT) cells, anti-tumor macrophages (e.g., M1 macrophages), B cells, and dendritic cells. In some embodiments, the targeting moieties specifically bind to a target (e.g., antigen, receptor) of interest and effectively recruit one of more immune cells. In some embodiments, the targets (e.g., antigens, receptors) of interest can be found on one or more tumor cells. In some embodiments, the present chimeric proteins or chimeric protein complexes, such as an chimeric proteins or chimeric protein complexes, such as an Fc-based chimeric protein complexes may recruit an immune cell, e.g., an immune cell that can kill and/or suppress a tumor cell, to a site of action (such as, by way of non-limiting example, the tumor microenvironment). In some embodiments, the targeting moieties specifically bind to a target (e.g., antigen, receptor) of interest which is part of a non-cellular structure. For clarity, the present invention includes, in embodiments, chimeric proteins or chimeric protein complexes, such as an chimeric proteins or chimeric protein complexes, such as an Fc-based chimeric protein complexes having one, or two, or three targeting moieties.

In some embodiments vectors encoding the present chimeric proteins or chimeric protein complexes, such as an chimeric proteins or chimeric protein complexes, such as an Fc-based chimeric protein complexes linked as a single nucleotide sequence to any of the linkers described herein are provided and may be used to prepare such chimeric proteins or chimeric protein complexes, such as an Fc-based chimeric protein complexes.

In some embodiments, the linker length allows for efficient binding of a targeting moiety and the signaling agent (e.g., IFNα2, IFNα1, IFNβ, or IL-1β or a variant thereof) to their receptors. For instance, in some embodiments, the linker length allows for efficient binding of one of the targeting moieties and the signaling agent to receptors on the same cell.

In some embodiments the linker length is at least equal to the minimum distance between the binding sites of one of the targeting moieties and the signaling agent to receptors on the same cell. In some embodiments the linker length is at least twice, or three times, or four times, or five times, or ten times, or twenty times, or 25 times, or 50 times, or one hundred times, or more the minimum distance between the binding sites of one of the targeting moieties and the signaling agent to receptors on the same cell.

As described herein, the linker length allows for efficient binding of one of the targeting moieties and the signaling agent to receptors on the same cell, the binding being sequential, e.g. targeting moiety/receptor binding preceding signaling agent/receptor binding.

In some embodiments, there are two linkers in a single chimera, each connecting the signaling agent to a targeting moiety. In various embodiments, the linkers have lengths that allow for the formation of a site that has a disease cell and an effector cell without steric hindrance that would prevent modulation of the either cell.

The invention contemplates the use of a variety of linker sequences. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In some embodiments, the linker may be designed using linker designing databases and computer programs such as those described in Chen et al., (2013), Adv Drug Deliv Rev. 65(10):1357-1369 and Crasto et al., (2000), Protein Eng. 13(5):309-312, the entire contents of which are hereby incorporated by reference. In various embodiments, the linker may be functional. For example, without limitation, the linker may function to improve the folding and/or stability, improve the expression, improve the pharmacokinetics, and/or improve the bioactivity of the present chimeric protein or chimeric protein complex, such as an Fc-based chimeric protein complex.

In some embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is a polypeptide. In some embodiments, the linker is greater than about 100 amino acids long. For example, the linker may be greater than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid.

In various embodiments, the linker is substantially comprised of glycine and serine residues (e.g. about 30%, or about 40%, or about 50%, or about 60%, or about 70%, or about 80%, or about 90%, or about 95%, or about 97% glycines and serines). For example, in some embodiments, the linker is (Gly₄Ser)ₙ, where n is from about 1 to about 8, e.g. 1, 2, 3, 4, 5, 6, 7, or 8 (SEQ ID NO: 10-SEQ ID NO: 17, respectively). In an embodiment, the linker sequence is GGSGGSGGGGSGGGGS (SEQ ID NO: 18). Additional illustrative linkers include, but are not limited to, linkers having the sequence LE, GGGGS (SEQ ID NO: 10), (GGGGS)ₙ (n=1-4) (SEQ ID NO:10-SEQ ID NO: 13), (Gly)₈ (SEQ ID NO: 19), (Gly)₆ (SEQ ID NO: 20), (EAAAK)ₙ (n=1-3) (SEQ ID NO: 21-SEQ ID NO: 23), A(EAAAK)ₙA (n=2-5) (SEQ ID NO: 24-SEQ ID NO:27), AEAAAKEAAAKA (SEQ ID NO: 24), A(EAAAK)₄ALEA(EAAAK)₄A (SEQ ID NO: 28), PAPAP (SEQ ID NO: 29), KESGSVSSEQLAQFRSLD (SEQ ID NO: 30), EGKSSGSGSESKST (SEQ ID NO: 31), GSAGSAAGSGEF (SEQ ID NO: 32), and (XP)ₙ, with X designating any amino acid, e.g., Ala, Lys, or Glu. In various embodiments, the linker is GGS.

In some embodiments, the linker is one or more of GGGSE (SEQ ID NO: 33), GSESG (SEQ ID NO: 34), GSEGS (SEQ ID NO: 35), GEGGSGEGSSGEGSSSEGGGSEGGGSEGGGSEGGS (SEQ ID NO: 36), and a linker of randomly placed G, S, and E every 4 amino acid intervals.

In some embodiments, the linker is a hinge region of an antibody (e.g., of IgG, IgA, IgD, and IgE, inclusive of subclasses (e.g., IgG1, IgG2, IgG3, and IgG4, and IgA1 and IgA2)). In various embodiments, the linker is a hinge region of an antibody (e.g., of IgG, IgA, IgD, and IgE, inclusive of subclasses (e.g., IgG1, IgG2, IgG3, and IgG4, and IgA1 and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains the sequence Cys-Pro-Pro-Cys (SEQ ID NO: 37), which when dimerized by disulfide bond formation, results in a cyclic octapeptide believed to act as a pivot, thus conferring flexibility. In various embodiments, the present linker comprises, one, or two, or three of the upper hinge region, the core region, and the lower hinge region of any antibody (e.g., of IgG, IgA, IgD, and IgE, inclusive of subclasses (e.g. IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In various embodiments, the linker of the present invention comprises one or more glycosylation sites.

In some embodiments, the linker is a synthetic linker such as PEG.

In various embodiments, the linker may be functional. For example, without limitation, the linker may function to improve the folding and/or stability, improve the expression, improve the pharmacokinetics, and/or improve the bioactivity of the present chimeric protein or chimeric protein complex, such as an Fc-based chimeric protein complex. In another example, the linker may function to target the chimeric protein or chimeric protein complex, such as an Fc-based chimeric protein complex to a particular cell type or location.

In various embodiments, the present chimeric protein or chimeric protein complex, such as an Fc-based chimeric protein complex may include one or more functional groups, residues, or moieties. In various embodiments, the one or more functional groups, residues, or moieties are attached or genetically fused to any of the signaling agents or targeting moieties described herein. In some embodiments, such functional groups, residues or moieties confer one or more desired properties or functionalities to the chimeric protein or chimeric protein complex, such as an Fc-based chimeric protein complex of the invention. Examples of such functional groups and of techniques for introducing them into the chimeric protein or chimeric protein complex, such as an Fc-based chimeric protein complex are known in the art, for example, see Remington's Pharmaceutical Sciences, 16th ed., Mack Publishing Co., Easton, Pa. (1980).

In various embodiments, each of the chimeric proteins or chimeric protein complexes, such as an Fc-based chimeric protein complexes may by conjugated and/or fused with another agent to extend half-life or otherwise improve pharmacodynamic and pharmacokinetic properties. In some embodiments, the chimeric proteins or chimeric protein complexes, such as an Fc-based chimeric protein complexes may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like.

In various embodiments, each of the individual chimeric proteins or chimeric protein complexes, such as an Fc-based chimeric protein complexes is fused to one or more of the agents described in BioDrugs (2015) 29:215-239, the entire contents of which are hereby incorporated by reference.

In some embodiments, the functional groups, residues, or moieties comprise a suitable pharmacologically acceptable polymer, such as poly(ethyleneglycol) (PEG) or derivatives thereof (such as methoxypoly(ethyleneglycol) or mPEG). In some embodiments, attachment of the PEG moiety increases the half-life and/or reduces the immunogenicity of the chimeric protein or chimeric protein complex, such as an Fc-based chimeric protein complex. Generally, any suitable form of pegylation can be used, such as the pegylation used in the art for antibodies and antibody fragments (including but not limited to single domain antibodies such as VHHs); see, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO04060965, the entire contents of which are hereby incorporated by reference. Various reagents for pegylation of proteins are also commercially available, for example, from Nektar Therapeutics, USA. In some embodiments, site-directed pegylation is used, in particular via a cysteine-residue (see, for example, Yang et al., Protein Engineering, 16, 10, 761-770 (2003), the entire contents of which is hereby incorporated by reference). In some embodiments, the chimeric protein or chimeric protein complex, such as an Fc-based chimeric protein complex of the invention is modified so as to suitably introduce one or more cysteine residues for attachment of PEG, or an amino acid sequence comprising one or more cysteine residues for attachment of PEG may be fused to the amino- and/or carboxy-terminus of the chimeric proteins or chimeric protein complexes, such as an Fc-based chimeric protein complexes, using techniques known in the art.

In some embodiments, the functional groups, residues, or moieties comprise N-linked or O-linked glycosylation. In some embodiments, the N-linked or O-linked glycosylation is introduced as part of a co-translational and/or post-translational modification.

In some embodiments, the functional groups, residues, or moieties comprise one or more detectable labels or other signal-generating groups or moieties. Suitable labels and techniques for attaching, using and detecting them are known in the art and, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels include moieties that can be detected using NMR or ESR spectroscopy. Such labeled VHHs and polypeptides of the invention may, for example, be used for in vitro, in vivo or in situ assays (including immunoassays known per se such as ELISA, RIA, EIA and other “sandwich assays,” etc.) as well as in vivo diagnostic and imaging purposes, depending on the choice of the specific label.

In some embodiments, the functional groups, residues, or moieties comprise a tag that is attached or genetically fused to the chimeric protein or chimeric protein complex, such as an Fc-based chimeric protein complex. In some embodiments, the chimeric protein or chimeric protein complex, such as an Fc-based chimeric protein complex may include a single tag or multiple tags. The tag for example is a peptide, sugar, or DNA molecule that does not inhibit or prevent binding of the chimeric protein or chimeric protein complex, such as an Fc-based chimeric protein complex to its target or any other antigen of interest such as tumor antigens. In various embodiments, the tag is at least about: three to five amino acids long, five to eight amino acids long, eight to twelve amino acids long, twelve to fifteen amino acids long, or fifteen to twenty amino acids long. Illustrative tags are described for example, in U.S. Patent Publication No. US2013/0058962. In some embodiment, the tag is an affinity tag such as glutathione-S-transferase (GST) and histidine (His) tag. In an embodiment, the chimeric protein or chimeric protein complex, such as an Fc-based chimeric protein complex comprises a His tag.

In some embodiments, the functional groups, residues, or moieties comprise a chelating group, for example, to chelate one of the metals or metallic cations. Suitable chelating groups, for example, include, without limitation, diethyl-enetriaminepentaacetic acid (DTPA) or ethylenediaminetetraacetic acid (EDTA).

In some embodiments, the functional groups, residues, or moieties comprise a functional group that is one part of a specific binding pair, such as the biotin-(strept)avidin binding pair. Such a functional group may be used to link the chimeric protein or chimeric protein complex, such as an Fc-based chimeric protein complex of the invention to another protein, polypeptide or chemical compound that is bound to the other half of the binding pair, i.e., through formation of the binding pair. For example, an chimeric protein or chimeric protein complex, such as an Fc-based chimeric protein complex of the invention may be conjugated to biotin, and linked to another protein, polypeptide, compound or carrier conjugated to avidin or streptavidin. For example, such a conjugated chimeric protein or chimeric protein complex, such as an Fc-based chimeric protein complex may be used as a reporter, for example, in a diagnostic system where a detectable signal-producing agent is conjugated to avidin or streptavidin. Such binding pairs may, for example, also be used to bind the chimeric protein or chimeric protein complex, such as an Fc-based chimeric protein complex to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the chimeric protein or chimeric protein complex, such as an Fc-based chimeric protein complex of the invention.

Methods for producing the chimeric proteins or chimeric protein complexes, such as an Fc-based chimeric protein complexes of the invention are described herein. For example, DNA sequences encoding the chimeric proteins or chimeric protein complexes, such as an Fc-based chimeric protein complexes of the invention (e.g., DNA sequences encoding the signaling agent (e.g., IFNα2, IFNα1, IFNβ, or IL-1β or a variant thereof) and the targeting moiety and the linker, or the polypeptide constituents of the chimeric proteins or chimeric protein complexes, such as an Fc-based chimeric protein complexes) can be chemically synthesized using methods known in the art. Synthetic DNA sequences can be ligated to other appropriate nucleotide sequences, including, e.g., expression control sequences, to produce gene expression constructs encoding the desired chimeric proteins or chimeric protein complexes, such as an Fc-based chimeric protein complexes. Accordingly, in various embodiments, the present invention provides for isolated nucleic acids comprising a nucleotide sequence encoding the chimeric protein or chimeric protein complex, such as an Fc-based chimeric protein complex of the invention, or the polypeptide subunits thereof.

Nucleic acids encoding the chimeric protein or chimeric protein complex, such as an Fc-based chimeric protein complex of the invention can be incorporated (ligated) into expression vectors, which can be introduced into host cells through transfection, transformation, or transduction techniques. For example, nucleic acids encoding the chimeric protein or chimeric protein complex, such as an Fc-based chimeric protein complex of the invention can be introduced into host cells by retroviral transduction. Illustrative host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the chimeric protein or chimeric protein complex, such as an Fc-based chimeric protein complex of the invention. Accordingly, in various embodiments, the present invention provides expression vectors comprising nucleic acids that encode the chimeric protein or chimeric protein complex, such as an Fc-based chimeric protein complex of the invention. In various embodiments, the present invention additional provides host cells comprising such expression vectors.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. In another example, if the engineered gene is to be expressed in eukaryotic host cells, e.g., CHO cells, it is first inserted into an expression vector containing for example, a suitable eukaryotic promoter, a secretion signal, enhancers, and various introns. The gene construct can be introduced into the host cells using transfection, transformation, or transduction techniques.

The chimeric protein or chimeric protein complex, such as an Fc-based chimeric protein complex of the invention can be produced by growing a host cell transfected with an expression vector encoding the chimeric protein or chimeric protein complex, such as an Fc-based chimeric protein complex under conditions that permit expression of the protein. Following expression, the protein can be harvested and purified using techniques well known in the art, e.g., affinity tags such as glutathione-S-transferase (GST) and histidine tags or by chromatography.

Accordingly, in various embodiments, the present invention provides for a nucleic acid encoding an chimeric protein or chimeric protein complex, such as an Fc-based chimeric protein complex of the present invention. In various embodiments, the present invention provides for a host cell comprising a nucleic acid encoding an chimeric protein or chimeric protein complex, such as an Fc-based chimeric protein complex of the present invention. In various embodiments, the present invention provides nucleic acid encoding an chimeric protein or chimeric protein complex, such as an Fc-based chimeric protein complex of the present invention which is suitable for production in a non-cellular system (e.g. in vitro transcription and/or in vitro translation).

In various embodiments, IFNα2, IFNα1, IFNβ, or IL-1β, its variant, or an chimeric protein or chimeric protein complex, such as an Fc-based chimeric protein complex comprising the IFNα2, IFNα1, IFNβ, or IL-1β or its variant may be expressed in vivo, for instance, in a patient. For example, in various embodiments, the IFNα2, IFNα1, IFNβ, or IL-1β, its variant, or an chimeric protein or chimeric protein complex, such as an Fc-based chimeric protein complex comprising the IFNα2, IFNα1, IFNβ, or IL-1β or its variant may administered in the form of nucleic acid which encodes for the IFNα2, IFNα1, IFNβ, or IL-1β or its variant or chimeric proteins or chimeric protein complexes, such as an Fc-based chimeric protein complexes comprising IFNα2, IFNα1, IFNβ, or IL-1β or its variant. In various embodiments, the nucleic acid is DNA or RNA. In some embodiments, the IFNα2, IFNα1, IFNβ, or IL-1β, its variant, or an chimeric protein or chimeric protein complex, such as an Fc-based chimeric protein complex comprising the IFNα2, IFNα1, IFNβ, or IL-1β or its variant is encoded by a modified mRNA, i.e. an mRNA comprising one or more modified nucleotides. In some embodiments, the modified mRNA comprises one or modifications found in U.S. Pat. No. 8,278,036, the entire contents of which are hereby incorporated by reference. In some embodiments, the modified mRNA comprises one or more of m5C, m5U, m6A, s2U, ψ, and 2′-O-methyl-U. In some embodiments, the present invention relates to administering a modified mRNA encoding one or more of the present chimeric proteins or chimeric protein complexes, such as an Fc-based chimeric protein complexes. In some embodiments, the present invention relates to gene therapy vectors comprising the same. In some embodiments, the present invention relates to gene therapy methods comprising the same. In various embodiments, the nucleic acid is in the form of an oncolytic virus, e.g. an adenovirus, reovirus, measles, herpes simplex, Newcastle disease virus or vaccinia.

The chimeric proteins or chimeric protein complexes, such as an Fc-based chimeric protein complexes described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, the compositions described herein are in the form of a pharmaceutically acceptable salt.

In various embodiments, the present invention pertains to pharmaceutical compositions comprising the chimeric proteins or chimeric protein complexes, such as an Fc-based chimeric protein complexes described herein and a pharmaceutically acceptable carrier or excipient. Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present invention includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, dessicated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule. In a further embodiment, the pharmaceutical composition is formulated in the form of a gelatin capsule. In yet another embodiment, the pharmaceutical composition is formulated as a liquid.

Where necessary, the inventive pharmaceutical compositions (and/or additional agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, any pharmaceutical compositions (and/or additional agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In some embodiments, the administering is effected orally. In another embodiment, the administration is by parenteral injection. The mode of administration can be left to the discretion of the practitioner, and depends in-part upon the site of the medical condition. In most instances, administration results in the release of any agent described herein into the bloodstream.

In one embodiment, the chimeric protein or chimeric protein complex, such as an Fc-based chimeric protein complex described herein is formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any chimeric proteins or chimeric protein complexes, such as an Fc-based chimeric protein complexes described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

The compositions provided herein, alone or in combination with other suitable components, can be made into aerosol formulations (i.e., “nebulized”) to be administered via inhalation. Aerosol formulations can be placed into pressurized acceptable propellants, such as dichlorodifluoromethane, propane, nitrogen, and the like.

Any inventive pharmaceutical compositions (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

It will be appreciated that the actual dose of the chimeric protein or chimeric protein complex, such as an Fc-based chimeric protein complex to be administered according to the present invention will vary according to the particular dosage form, and the mode of administration. Many factors that may modify the action of the chimeric protein or chimeric protein complex, such as an Fc-based chimeric protein complex (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In some embodiments, a suitable dosage of the chimeric protein or chimeric protein complex, such as an Fc-based chimeric protein complex is in a range of about 0.01 μg/kg to about 100 mg/kg of body weight of the subject, about 0.01 μg/kg to about 10 mg/kg of body weight of the subject, or about 0.01 μg/kg to about 1 mg/kg of body weight of the subject for example, about 0.01 μg/kg, about 0.02 μg/kg, about 0.03 μg/kg, about 0.04 μg/kg, about 0.05 μg/kg, about 0.06 μg/kg, about 0.07 μg/kg, about 0.08 μg/kg, about 0.09 μg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, or about 100 mg/kg body weight, inclusive of all values and ranges therebetween.

Individual doses of the chimeric protein or chimeric protein complex, such as an Fc-based chimeric protein complex can be administered in unit dosage forms (e.g., tablets, capsules, or liquid formulations) containing, for example, from about 1 μg to about 100 mg, from about 1 μg to about 90 mg, from about 1 μg to about 80 mg, from about 1 μg to about 70 mg, from about 1 μg to about 60 mg, from about 1 μg to about 50 mg, from about 1 μg to about 40 mg, from about 1 μg to about 30 mg, from about 1 μg to about 20 mg, from about 1 μg to about 10 mg, from about 1 μg to about 5 mg, from about 1 μg to about 3 mg, from about 1 μg to about 1 mg per unit dosage form, or from about 1 μg to about 50 μg per unit dosage form. For example, a unit dosage form can be about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29, about 30 μg, about 35 μg, about 40 μg, about 45 μg, about 50 μg, about 60 μg, about 70 μg, about 80 μg, about 90 μg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all values and ranges therebetween.

In one embodiment, the chimeric protein or chimeric protein complex, such as an Fc-based chimeric protein complex is administered at an amount of from about 1 μg to about 100 mg daily, from about 1 μg to about 90 mg daily, from about 1 μg to about 80 mg daily, from about 1 μg to about 70 mg daily, from about 1 μg to about 60 mg daily, from about 1 μg to about 50 mg daily, from about 1 μg to about 40 mg daily, from about 1 μg to about 30 mg daily, from about 1 μg to about 20 mg daily, from about 01 μg to about 10 mg daily, from about 1 μg to about 5 mg daily, from about 1 μg to about 3 mg daily, or from about 1 μg to about 1 mg daily. In various embodiments, the chimeric protein or chimeric protein complex, such as an Fc-based chimeric protein complex is administered at a daily dose of about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29, about 30 μg, about 35 μg, about 40 μg, about 45 μg, about 50 μg, about 60 μg, about 70 μg, about 80 μg, about 90 μg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, inclusive of all values and ranges therebetween.

In accordance with certain embodiments of the invention, the pharmaceutical composition comprising the chimeric protein or chimeric protein complex, such as an Fc-based chimeric protein complex may be administered, for example, more than once daily (e.g., about two times, about three times, about four times, about five times, about six times, about seven times, about eight times, about nine times, or about ten times daily), about once per day, about every other day, about every third day, about once a week, about once every two weeks, about once every month, about once every two months, about once every three months, about once every six months, or about once every year. In an embodiment, the pharmaceutical composition comprising the chimeric protein or chimeric protein complex, such as an Fc-based chimeric protein complex is administered about three times a week.

In various embodiments, the present chimeric protein or chimeric protein complex, such as an Fc-based chimeric protein complex may be administered for a prolonged period. For example, the chimeric protein or chimeric protein complex, such as an Fc-based chimeric protein complex may be administered as described herein for at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks. For example, the chimeric protein or chimeric protein complex, such as an Fc-based chimeric protein complex may be administered for 12 weeks, 24 weeks, 36 weeks or 48 weeks. In some embodiments, the chimeric protein or chimeric protein complex, such as an Fc-based chimeric protein complex is administered for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. n some embodiments, the chimeric protein or chimeric protein complex, such as an Fc-based chimeric protein complex may be administered for at least about 1 year, at least about 2 years, at least about 3 years, at least about 4 years, or at least about 5 years.

In various embodiments, the pharmaceutical composition of the present invention is co-administered in conjunction with additional therapeutic agent(s). Co-administration can be simultaneous or sequential.

In one embodiment, the additional therapeutic agent and the chimeric protein or chimeric protein complex, such as an Fc-based chimeric protein complex of the present invention are administered to a subject simultaneously. The term “simultaneously” as used herein, means that the additional therapeutic agent and the chimeric protein or chimeric protein complex, such as an Fc-based chimeric protein complex are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the chimeric protein or chimeric protein complex, such as an Fc-based chimeric protein complex can be by simultaneous administration of a single formulation (e.g., a formulation comprising the additional therapeutic agent and the chimeric protein or chimeric protein complex, such as an Fc-based chimeric protein complex) or of separate formulations (e.g., a first formulation including the additional therapeutic agent and a second formulation including the chimeric protein or chimeric protein complex, such as an Fc-based chimeric protein complex).

Co-administration does not require the therapeutic agents to be administered simultaneously, if the timing of their administration is such that the pharmacological activities of the additional therapeutic agent and the chimeric protein overlap in time, thereby exerting a combined therapeutic effect. For example, the additional therapeutic agent and the chimeric protein or chimeric protein complex, such as an Fc-based chimeric protein complex can be administered sequentially. The term “sequentially” as used herein means that the additional therapeutic agent and the chimeric protein or chimeric protein complex, such as an Fc-based chimeric protein complex are administered with a time separation of more than about 60 minutes. For example, the time between the sequential administration of the additional therapeutic agent and the chimeric protein or chimeric protein complex, such as an Fc-based chimeric protein complex can be more than about 60 minutes, more than about 2 hours, more than about 5 hours, more than about 10 hours, more than about 1 day, more than about 2 days, more than about 3 days, more than about 1 week apart, more than about 2 weeks apart, or more than about one month apart. The optimal administration times will depend on the rates of metabolism, excretion, and/or the pharmacodynamic activity of the additional therapeutic agent and the chimeric protein or chimeric protein complex, such as an Fc-based chimeric protein complex being administered. Either the additional therapeutic agent or the chimeric protein or chimeric protein complex, such as an Fc-based chimeric protein complex cell may be administered first.

Co-administration also does not require the therapeutic agents to be administered to the subject by the same route of administration. Rather, each therapeutic agent can be administered by any appropriate route, for example, parenterally or non-parenterally.

In some embodiments, the chimeric protein or chimeric protein complex, such as an Fc-based chimeric protein complex described herein acts synergistically when co-administered with another therapeutic agent. In such embodiments, the chimeric protein or chimeric protein complex, such as an Fc-based chimeric protein complex and the additional therapeutic agent may be administered at doses that are lower than the doses employed when the agents are used in the context of monotherapy.

In some embodiments, the present invention pertains to chemotherapeutic agents as additional therapeutic agents. For example, without limitation, such combination of the present chimeric proteins or chimeric protein complexes, such as an Fc-based chimeric protein complexes and chemotherapeutic agent find use in the treatment of cancers, as described elsewhere herein. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

In some embodiments, the chimeric protein or chimeric protein complex, such as an Fc-based chimeric protein complex described herein, includes derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of tunicamycin, etc.

In still other embodiments, the chimeric protein or chimeric protein complex, such as an Fc-based chimeric protein complex described herein further comprises a cytotoxic agent, comprising, in illustrative embodiments, a toxin, a chemotherapeutic agent, a radioisotope, and an agent that causes apoptosis or cell death. Such agents may be conjugated to a composition described herein.

The chimeric protein or chimeric protein complex, such as an Fc-based chimeric protein complex described herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

Illustrative cytotoxic agents include, but are not limited to, methotrexate, aminopterin, 6-mercaptopurine, 6-thioguanine, cytarabine, 5-fluorouracil decarbazine; alkylating agents such as mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin (formerly daunomycin), doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimytotic agents such as the vinca alkaloids, vincristine and vinblastine. Other cytotoxic agents include paclitaxel (taxol), ricin, Pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, asparaginase, corticosteroids, mytotane (O,P′-(DDD)), interferons, and mixtures of these cytotoxic agents.

Further cytotoxic agents include, but are not limited to, chemotherapeutic agents such as carboplatin, cisplatin, paclitaxel, gemcitabine, calicheamicin, doxorubicin, 5-fluorouracil, mitomycin C, actinomycin D, cyclophosphamide, vincristine, bleomycin, VEGF antagonists, EGFR antagonists, platins, taxols, irinotecan, 5-fluorouracil, gemcytabine, leucovorine, steroids, cyclophosphamide, melphalan, vinca alkaloids (e.g., vinblastine, vincristine, vindesine and vinorelbine), mustines, tyrosine kinase inhibitors, radiotherapy, sex hormone antagonists, selective androgen receptor modulators, selective estrogen receptor modulators, PDGF antagonists, TNF antagonists, IL-1 antagonists, interleukins (e.g. IL-12 or IL-2), IL-12R antagonists, Toxin conjugated monoclonal antibodies, tumor antigen specific monoclonal antibodies, Erbitux, Avastin, Pertuzumab, anti-CD20 antibodies, Rituxan, ocrelizumab, ofatumumab, DXL625, HERCEPTIN®, or any combination thereof. Toxic enzymes from plants and bacteria such as ricin, diphtheria toxin and Pseudomonas toxin may be conjugated to the therapeutic agents (e.g. antibodies) to generate cell-type-specific-killing reagents (Youle, et al., Proc. Nat'l Acad. Sci. USA 77:5483 (1980); Gilliland, et al., Proc. Nat'l Acad. Sci. USA 77:4539 (1980); Krolick, et al., Proc. Nat'l Acad. Sci. USA 77:5419 (1980)).

Other cytotoxic agents include cytotoxic ribonucleases as described by Goldenberg in U.S. Pat. No. 6,653,104. Embodiments of the invention also relate to radioimmunoconjugates where a radionuclide that emits alpha or beta particles is stably coupled to the chimeric protein or chimeric protein complex, such as an Fc-based chimeric protein complex, with or without the use of a complex-forming agent. Such radionuclides include beta-emitters such as Phosphorus-32, Scandium-47, Copper-67, Gallium-67, Yttrium-88, Yttrium-90, Iodine-125, Iodine-131, Samarium-153, Lutetium-177, Rhenium-186 or Rhenium-188, and alpha-emitters such as Astatine-211, Lead-212, Bismuth-212, Bismuth-213 or Actinium-225.

Illustrative detectable moieties further include, but are not limited to, horseradish peroxidase, acetylcholinesterase, alkaline phosphatase, beta-galactosidase and luciferase. Further illustrative fluorescent materials include, but are not limited to, rhodamine, fluorescein, fluorescein isothiocyanate, umbelliferone, dichlorotriazinylamine, phycoerythrin and dansyl chloride. Further illustrative chemiluminescent moieties include, but are not limited to, luminol. Further illustrative bioluminescent materials include, but are not limited to, luciferin and aequorin. Further illustrative radioactive materials include, but are not limited to, Iodine-125, Carbon-14, Sulfur-35, Tritium and Phosphorus-32.

In some embodiments, inclusive, without limitation, of autoimmune applications, the additional therapeutic agent is an immunosuppressive agent that is an anti-inflammatory agent such as a steroidal anti-inflammatory agent or a non-steroidal anti-inflammatory agent (NSAID). Steroids, particularly the adrenal corticosteroids and their synthetic analogues, are well known in the art. Examples of corticosteroids useful in the present invention include, without limitation, hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that may be used in the present invention, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin. In some embodiments, the immunosupressive agent may be cytostatics such as alkylating agents, antimetabolites (e.g., azathioprine, methotrexate), cytotoxic antibiotics, antibodies (e.g., basiliximab, daclizumab, and muromonab), anti-immunophilins (e.g., cyclosporine, tacrolimus, sirolimus), inteferons, opioids, TNF binding proteins, mycophenolates, and small biological agents (e.g., fingolimod, myriocin). Additional anti-inflammatory agents are described, for example, in U.S. Pat. No. 4,537,776, the entire contents of which is incorporated by reference herein.

In some embodiments, the chimeric protein or chimeric protein complex, such as an Fc-based chimeric protein complex is used in a method of treating multiple sclerosis in combination with one or more of the disease modifying therapies (DMTs) described herein (e.g. the agents of Table A). In some embodiments, the present invention provides an improved therapeutic effect as compared to use of one or more of the DMTs described herein (e.g. the agents listed in Table A below) without the one or more disclosed binding agent. In an embodiment, the combination of the chimeric protein or chimeric protein complex, such as an Fc-based chimeric protein complex and the one or more DMTs produces synergistic therapeutic effects.

Illustrative disease modifying therapies include, but are not limited to:

TABLE A

| Generic Name | Branded Name/Company | Frequency/Route of Delivery/Usual Dose |
|---|---|---|
| teriflunomide | AUBAGIO (GENZYME) | Every day; pill taken orally; 7 mg or 14 mg. |
| interferon beta-1a | AVONEX (BIOGEN IDEC) | Once a week; intramuscular (into the muscle) |
| | | injection; 30 mcg |
| interferon beta-1b | BETASERON (BAYER | Every other day; subcutaneous (under the skin) |
| | HEALTHCARE | injection; 250 mcg. |
| | PHARMACEUTICALS, INC.) | |
| glatiramer acetate | COPAXONE (TEVA | Every day; subcutaneous (under the skin) |
| | NEUROSCIENCE) | injection; 20 mg (20,000 mcg) OR Three times a |
| | | week; subcutaneous (under the skin) injection; 40 |
| | | mg (40,000 mcg) |
| interferon beta-1b | EXTAVIA (NOVARTIS | Every other day; subcutaneous (under the skin) |
| | PHARMACEUTICALS CORP.) | injection; 250 mcg. |
| fingolimod | GILENYA (NOVARTIS | Every day; capsule taken orally; 0.5 mg. |
| | PHARMACEUTICALS CORP.) | |
| Alemtuzumab (anti-CD52 | LEMTRADA (GENZYME) | Intravenous infusion on five consecutive days, |
| monoclonal antibody) | | followed by intravenous infusion on three |
| | | consecutive days one year later (12 mg) |
| mitoxantrone | NOVANTRONE (EMD | Four times a year by IV infusion in a medical |
| | SERONO) | facility. Lifetime cumulative dose limit of |
| | | approximately 8-12 doses over 2-3 years (140 |
| | | mg/m2). |
| pegylated interferon beta-1a | PLEGRIDY (BIOGEN IDEC) | Every 14 days; subcutaneous (under the skin) |
| | | injection; 125 mcg |
| interferon beta-1a | REBIF (EMD SERONO, INC.) | Three times a week; subcutaneous (under the |
| | | skin) injection; 44 mcg |
| dimethyl fumarate (BG-12) | TECFIDERA (BIOGEN IDEC) | Twice a day; capsule taken orally; 120 mg for one |
| | | week and 240 mg therafter |
| Natalizumab (humanized | TYSABRI (BIOGEN IDEC) | Every four weeks by IV infusion in a registered |
| monoclonal antibody VLA-4 | | infusion facility; 300 mg |
| antagonist) | | |

DMTs in Development

| | | |
|---|---|---|
| Amiloride (targets Acid- | PAR PHARMACEUTICAL, | Oral |
| sensing ion channel-1 | PERRIGO COMPANY, | |
| Epithelial sodium channel | SIGMAPHARM | |
| Na+/H+ exchanger) | LABORATORIES | |
| ATX-MS-1467 (targets Major | APITOPE/MERCK SERONO | Intradermal Subcutaneous |
| histocompatibility complex | | |
| class II T cell responses to | | |
| myelin basic protein) | | |
| BAF312 (targets | NOVARTIS PHARMA | Oral |
| Sphingosine 1-phosphate | | |
| (S1P) receptor subtypes | | |
| S1P1 and S1P5 B cell | | |
| distrubution T cell | | |
| distribution) | | |
| BGC20-0134 (targets | BTG PLC | Oral |
| Proinflammatory and anti- | | |
| inflammatory cytokines) | | |
| BIIB033 (targets LINGO-1 | BIOGEN | Intravenous infusion used in Phase I and Phase II |
| (“leucine-rich repeat and | | trials Subcutaneous injection used in Phase I trial |
| immunoglobulin-like domain- | | |
| containing, Nogo receptor- | | |
| interacting protein”)) | | |
| Cladribine (targets CD4+ T | MERCK SERONO | Oral |
| cells DNA synthesis and | | |
| repair E-selectin Intracellular | | |
| adhesion molecule-1 Pro- | | |
| inflammatory cytokines | | |
| interleukin 2 and interleukin | | |
| 2R Pro-inflammatory | | |
| cytokines interleukin 8 and | | |
| RANTES Cytokine secretion | | |
| Monocyte and lymphocyte | | |
| migration) | | |
| Cyclophosphamide (targets | BAXTER HEALTHCARE | Oral, monthly intravenous pulses |
| T cells, particularly CD4+ | CORPORATION | |
| helper T cells B cells) | | |
| Daclizumab (humanized | BIOGEN IDEC/ABBVIE | Projected to be IM injection once monthly |
| monoclonal antibody | BIOTHERAPEUTICS | |
| targeting CD25 Immune | | |
| modulator of T cells) | | |
| Dalfampridine (targets | ACORDA THERAPEUTICS/ | One tablet every 12 hours (extended release), 10 |
| Voltage-gated potassium | BIOGEN IDEC | mg twice a day |
| channels | | |
| Degenerin/epithelial sodium | | |
| channels L-type calcium | | |
| channels that contain | | |
| subunit Cavbeta3) | | |
| Dronabinol (targets | ABBVIE INC. | Oral |
| Cannabinoid receptor CB1 | | |
| Cannabinoid receptor CB2) | | |
| Firategrast (targets | GLAXOSMITHKLINE | Oral |
| Alpha4beta1 integrin) | | |
| GNbAC1MSRV-Env (targets | GENEURO SA/SERVER | Intravenous infusion |
| envelope protein of the MS- | | |
| associated retrovirus) | | |
| Idebenone (targets Reactive | SANTHERA | Oral Dose in clinical trial for PPMS is 2250 mg per |
| oxygen species) | PHARMACEUTICALS | day (750 mg dose, 3 times per day) |
| Imilecleucel-T (targets | OPEXA THERAPEUTICS/ | Subcutaneous Given 5 times per year, according |
| Myelin-specific, autoreactive | MERCK SERONO | to information from the manufacturer |
| T cells) | | |
| Laquinimod | TEVA | Projected to be 0.6 mg or 1.2 mg oral tablet taken |
| | | daily |
| Masitinib (targets KIT (a | AB SCIENCE | Oral |
| stem cell factor, also called | | |
| c-KIT) receptor as well as | | |
| select other tyrosine kinases | | |
| Mast cells) | | |
| MEDI-551 (targets CD19, a | MEDIMMUNE | Intravenous Subcutaneous |
| B cell-specific antigen that is | | |
| part of the B cell receptor | | |
| complex and that functions | | |
| in determining the threshold | | |
| for B cell activation B cells | | |
| Plasmablasts, B cells that | | |
| express CD19 (but not | | |
| CD20) and that secrete large | | |
| quantities of antibodies; | | |
| depletion of plasmablasts | | |
| may be useful in | | |
| autoimmune diseases | | |
| involving pathogenic | | |
| autoantibodies) | | |
| Minocycline (targets T cells | VARIOUS | Oral Available as pellet-filled capsules and an oral |
| Microglia Leukocyte | | suspension |
| migration Matrix | | |
| metalloproteinases) | | |
| MIS416 (targets Innate | INNATE | Intravenous |
| immune system Pathogen- | IMMUNOTHERAPEUTICS | |
| associated molecular pattern | | |
| recognition receptors of the | | |
| innate immune system | | |
| Myeloid cells of the innate | | |
| immune system, which might | | |
| be able to remodel the | | |
| deregulated immune system | | |
| activity that occurs in SPMS) | | |
| Mycophenolate mofetil | MANUFACTURED BY | Oral |
| (targets Purine synthesis) | GENENTECH | |
| Naltrexone (targets Opioid | VARIOUS | Given at low doses (3 to 4.5 mg per day) in oral |
| receptors Toll-like receptor | | form as “Low-dose naltrexone” (or “LDN”) |
| 4) | | |
| Ocrelizumab and | ROCHE/GSK | Projected to be IV infusion |
| Ofatumumab (humanized | | |
| monoclonal antibodies | | |
| targeting CD20 B cell | | |
| suppression | | |
| ONO-4641 (targets | ONO PHARMACEUTICAL CO. | Oral |
| Sphingosine 1-phosphate | | |
| receptor) | | |
| Phenytoin (targets Sodium | PFIZER | Intravenous Intramuscular (less favored option) |
| channels) | | Oral |
| Ponesimod | ACTELION | To be determined |
| Raltegravir (targets | MERCK | Oral 400 mg tablet twice daily, according to |
| Retroviral integrase | | information from the manufacturer |
| Herpesvirus DNA packaging | | |
| terminase) | | |
| RHB-104 | REDHILL BIOPHARMA | 95 mg clarithromycin, 45 mg rifabutin, and 10 mg |
| | LIMITED | clofazimine |
| Riluzole (targets | COVIS PHARMA/SANOFI | Oral |
| Glutamatergic | | |
| neurotransmission | | |
| Glutamate uptake and | | |
| release Voltage-gated | | |
| sodium channels Protein | | |
| kinase C) | | |

The invention also provides kits for the administration of any agent described herein (e.g. the chimeric protein or chimeric protein complex, such as an Fc-based chimeric protein complex with or without various additional therapeutic agents). The kit is an assemblage of materials or components, including at least one of the inventive pharmaceutical compositions described herein. Thus, in some embodiments, the kit contains at least one of the pharmaceutical compositions described herein.

The exact nature of the components configured in the kit depends on its intended purpose. In one embodiment, the kit is configured for the purpose of treating human subjects.

Instructions for use may be included in the kit. Instructions for use typically include a tangible expression describing the technique to be employed in using the components of the kit to effect a desired outcome, such as to treat cancer. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials and components assembled in the kit can be provided to the practitioner stored in any convenience and suitable ways that preserve their operability and utility. For example, the components can be provided at room, refrigerated or frozen temperatures. The components are typically contained in suitable packaging materials. In various embodiments, the packaging material is constructed by well-known methods, preferably to provide a sterile, contaminant-free environment. The packaging material may have an external label which indicates the contents and/or purpose of the kit and/or its components.

### Definitions

As used herein, “a,” “an,” or “the” can mean one or more than one.

Unless specifically stated or obvious from context, as used herein, the term “or” is understood to be inclusive and covers both “or” and “and”.

Further, the term “about” when used in connection with a referenced numeric indication means the referenced numeric indication plus or minus up to 10% of that referenced numeric indication, e.g., within (plus or minus) 10%, 9%, 8%, 7%, 6%, 5%, 4%, 3%, 2%, 1%, 0.5%, 0.1%, 0.05%, or 0.01% of the stated value. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the 1050 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein. This invention is further illustrated by the following non-limiting examples.

### EXAMPLES

“AFN” is used occasionally herein to refer to an interferon-based chimeric protein or chimeric protein complex (as indicated herein).

### Example 1: FLT3L Fc AFNs

In this Example, we designed and evaluated AFNs based on FMS-like tyrosine kinase 3 ligand (FLT3L) for targeting of FLT3-positive cells.

### Constructs

○ FLT3L-S-20*GGS-Fc3 (P-1623)
(SEQ ID NO: 40)
TQDCSFQHSPISSDFAVKIRELSDYLLQDYPVTVASNLQDEELCGGLWR
LVLAQRWMERLKTVAGSKMQGLLERVNTEIHFVTKCAFQPPPSCLRFVQ
TNISRLLQETSEQLVALKPWITRQNFSRCLELQCQPDSSTLPPPWSPRP
LEATAPTAPQPSGGSGGSGGSGGSGGSGGSGGSGGSGGSGGSGGSGGSG
GSGGSGGSGGSGGSGGSGGSGGSDKTHTCPPCPAPEAAGGPSVFLFPPK
PKDTLMISRTPEVTCVVVDVSHEDPEVKFNWYVDGVEVHNAKTKPREEQ
YNSTYRWSVLTVLHQDWLNGKEYKCQVSNKALPAPIEKTISKAKGQPRE
PQVCTLPPSRDELTKNQVSLSCAVKGFYPSDIAVEWESNGQPENNYKTT
PPVLDSDGSFFLVSKLTVDKSRWQQGNVFSCSVMHEALHNHYTQKSLSL
SPGK
○ Fc4-20*GGS-IFNa2_R149A (P-1414)
(SEQ ID NO: 41)
DKTHTCPPCPAPEAAGGPSVFLFPPKPKDTLMISRTPEVTCVVVDVSHE
DPEVKFNWYVDGVEVHNAKTKPREEQYNSTYRWSVLTVLHQDWLNGKEY
KCQVSNKALPAPIEKTISKAKGQPREPQVYTLPPCRDELTKNQVSLWCL
VKGFYPSDIAVEWESNGQPENNYKTTPPVLDSDGSFFLYSKLTVDKSRW
QQGNVFSCSVMHEALHNHYTQKSLSLSPGKGGSGGSGGSGGSGGSGGSG
GSGGSGGSGGSGGSGGSGGSGGSGGSGGSGGSGGSGGSGGSCDLPQTHS
LGSRRTLMLLAQMRKISLFSCLKDRHDFGFPQEEFGNQFQKAETIPVLH
EMIQQIFNLFSTKDSSAAWDETLLDKFYTELYQQLNDLEACVIQGVGVE
ETPLMKEDSILAVRKYFQRITLYLKEKKYSPCAWEVVRAEIMASFSLST
NLQESLRSKE

### Production and Purification of FLT3L-Based AFN

Constructs FLT3L-20*GGS-Fc3 and Fc4-20*GGS-IFNα2_R149A were combined to an AFN variant with a configuration outlined in FIG. 7B, and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Biological Activity: STAT1 Phosphorylation in Transient Transfected Hek293T Cells

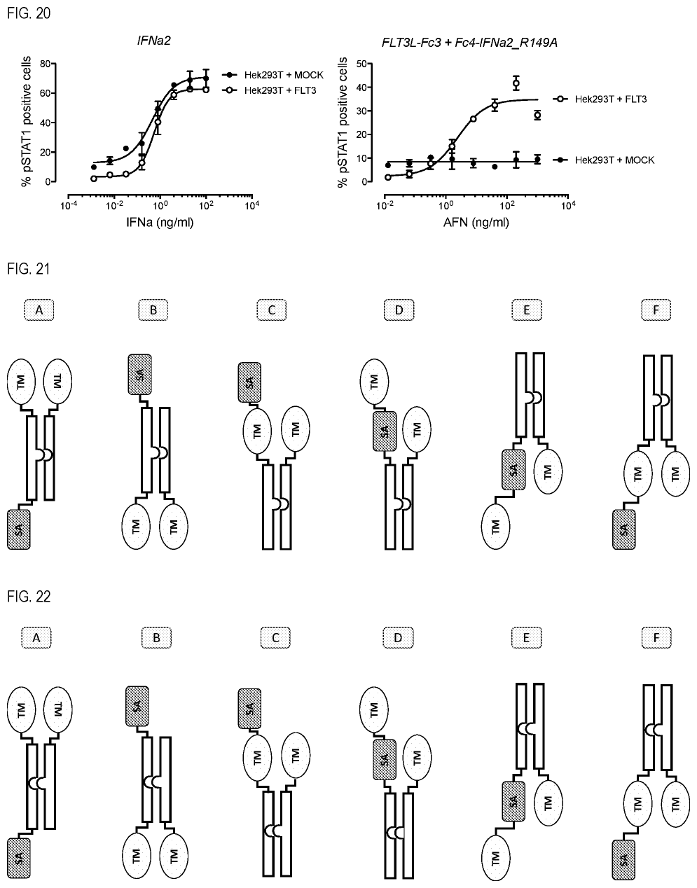

Hek293T cells were transiently transfected with a FLT3 expression plasmid or an empty vector (MOCK). Two days after transfection, cells were stimulated with a serial dilution (as indicated) wild type IFNα2 or FLT3L-Fc-AFN for 15 minutes at 37° C. After fixation (10 minutes, 37° C., Fix Buffer I; BD Biosciences), permeabilization (30 minutes, on ice, Perm III Buffer I; BD Biosciences) and washing, cells were stained with anti-STAT1 pY701 Ab (BD Biosciences). Samples were acquired with a MACSQuant X instrument (Miltenyi Biotec) and analysed using the FlowLogic software (Miltenyi Biotec). Data in FIG. 20 clearly illustrate that the FLT3L Fc AFN is only capable to induce STAT1 phosphorylation in FLT3 and not MOCK transfected cells, thereby illustrating that targeting using this ligand is possible. Of note, FLT3 or MOCK transfected cells are equally sensitive to wild type IFNα2.

### Example 2: Split FLT3L Fc AFN Variants

In this example, we evaluated the potential of the Fc formatted IFN based AFNs targeted by FLT3L aiming to selectively activate FLT3 positive cells using constructs in which one copy of FLT3L is presented on each Fc-chain. The possibilities for such split FLT3L Fc constructs in a ‘knob-in-hole’ Fc context are show in FIGS. 21A-F and FIGS. 22A-F. For the exemplified heterodimers (see for structure FIG. 21A), the FLT3L sequence was cloned N-terminal of both Fc arms, resulting in a so-called ‘split’ configuration. The first FLT3L sequence was, in the pcDNA 3.4 expression vector, fused to the human IgG1 Fc sequence containing the L234A_L235A_K322Q effector mutations and the ‘hole’ modifications Y349C_T366S_L368A_Y407V. The second AFN partner, also cloned in the pcDNA 3.4 vector, consists of the fusion between the FLT3L sequence, the human IgG1 Fc sequence containing the L234A_L235A_K322Q effector mutations and the ‘knob’ modifications S354C_T366W and the hIFNα2 sequence with the AFN mutation R149A and deletion of the O-glycosylation site at T106 by mutation to E. Three variants were tested: in a first variant, sequence of FLT3L amino acid (aa) 27-184 was fused via a 5*GGS linker to the Fc arms. The same FLT3L sequence was directly fused to the Fc arms in a second variant. In a third variant, a combination of FLT3L amino acids 27-160 and a 5*GGS linker was used. In each variant, residue L27 in the FLT3-L dimerization interface was further optionally mutated to D. This resulted in twelve different sequences (see below).

To produce these ‘knob-in-hole’ Fc AFNs, a combination of both ‘hole’ and ‘knob’ plasmids was transfected in ExpiCHO cells (ThermoFisher) according to the manufacturer's instructions. Seven days post transfection, recombinant proteins were purified using protein A spin plates (ThermoFisher), quantified and purity tested using SDS-PAGE. The SDS-PAGE under non-reducing conditions showed a more homogeneous profile for the constructs containing the L27D mutation.

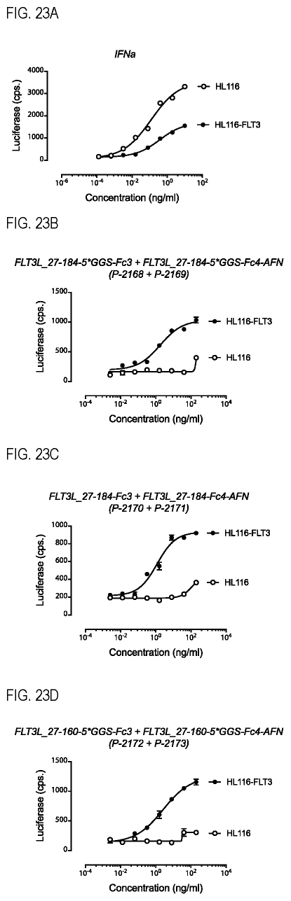

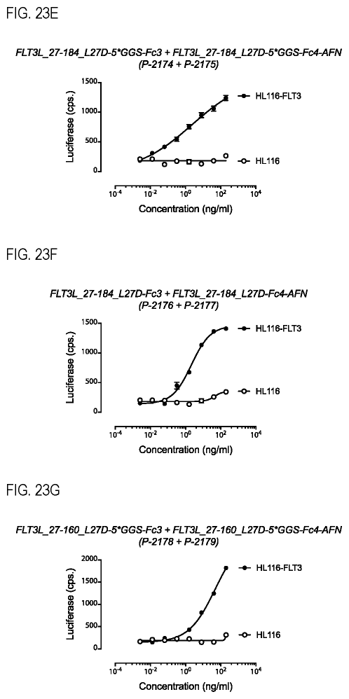

Biological activity of resulting proteins was measured on parental HL116 cells (an IFN responsive cell-line stably transfected with a p6-16 luciferase reporter) and the derived, stably transfected HL116-FLT3 cells. Cells were seeded overnight and stimulated for 6 hours with a serial dilution FLT3L AFNs. Luciferase activity was measured on an EnSight Multimode Plate Reader (Perkin Elmer). Data in FIG. 23A-G clearly illustrate that (i) both parental HL116 and HL116-FLT3 cells are comparable responsive to wild type IFNα2, (ii) all FLT3L AFNs are clearly more active on targeted (FLT3 expressing) cells vs non-targeted cells, (iii) no clear differences in IFN-like signaling between FLT3L variants, and (iv) the L27D mutation has no apparent effect on signaling on both targeted or non-targeted cells.

### Sequences:

| | |
|---|---|
| 1. | P-2168: FLT3L_27-184-S-5*GGS-Fc3 |
| | (SEQ ID NO: 46) |
| | TQDCSFQHSPISSDFAVKIRELSDYLLQDYPVTVASNLQDEELCGGLWRLVLAQRWMERLKTVAGSKMQGLLE |
| | RVNTEIHFVTKCAFQPPPSCLRFVQTNISRLLQETSEQLVALKPWITRQNFSRCLELQCQPDSSTLPPPWSPRPL |
| | EATAPTAPQPSGGSGGSGGSGGSGGSDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLHQDWLNGKEYKCQVSNKALPAPIEKT |
| | ISKAKGQPREPQVCTLPPSRDELTKNQVSLSCAVKGFYPSDIAVEWESNGQPENNYKTTPPVLDSDGSFFL |
| | VSKLTVDKSRWQQGNVFSCSVMHEALHNHYTQKSLSLSPGK |
| 2. | P-2169: FLT3L_27-184-S-5*GGS-Fc4-AFN |
| | (SEQ ID NO: 47) |
| | TQDCSFQHSPISSDFAVKIRELSDYLLQDYPVTVASNLQDEELCGGLWRLVLAQRWMERLKIVAGSKMQGLLE |
| | RVNTEIHFVTKCAFQPPPSCLRFVQTNISRLLQETSEQLVALKPWITRQNFSRCLELQCQPDSSTLPPPWSPRPL |
| | EATAPTAPQPSGGSGGSGGSGGSGGSDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLHQDWLNGKEYKCQVSNKALPAPIEKT |
| | ISKAKGQPREPQVYTLPPCRDELTKNQVSLWCLVKGFYPSDIAVEWESNGQPENNYKTTPPVLDSDGSFFL |
| | YSKLTVDKSRWQQGNVFSCSVMHEALHNHYTQKSLSLSPGKGGSGGSGGSGGSGGSGGSGGSGGSGG |
| | SGGSGGSGGSGGSGGSGGSGGSGGSGGSGGSGGSCDLPQTHSLGSRRTLMLLAQMRKISLFSCLKDR |
| | HDFFPQEEFGNQFQKAETIPVLHEMIQQIFNLFSTKDSSAAWDETLLDKFYTELYQQLNDLEACVIQGVGV |
| | EETPLMKEDSILAVRKYFQRITLYLKEKKYSPCAWEVVRAEIMASFSLSTNLQESLRSKE |
| 3. | P-2170: FLT3L_27-184-S-Fc3 |
| | (SEQ ID NO: 48) |
| | TQDCSFQHSPISSDFAVKIRELSDYLLQDYPVIVASNLQDEELCGGLWRLVLAQRWMERLKIVAGSKMQGLLE |
| | RVNTEIHFVTKCAFQPPPSCLRFVQTNISRLLQETSEQLVALKPWITRQNFSRCLELQCQPDSSTLPPPWSPRPL |
| | EATAPTAPQPSDKTHTCPPCPAPEAAGGPSVFLFPPKPKDTLMISRTPEVTCVVVDVSHEDPEVKFNWYVD |
| | GVEVHNAKTKPREEQYNSTYRVVSVLTVLHQDWLNGKEYKCQVSNKALPAPIEKTISKAKGQPREPQVCTL |
| | PPSRDELTKNQVSLSCAVKGFYPSDIAVEWESNGQPENNYKTTPPVLDSDGSFFLVSKLTVDKSRWQQGN |
| | VFSCSVMHEALHNHYTQKSLSLSPGK |
| 4. | P-2171: FLT3L_27-184- S-Fc4-AFN |
| | (SEQ ID NO: 49) |
| | TQDCSFQHSPISSDFAVKIRELSDYLLQDYPVIVASNLQDEELCGGLWRLVLAQRWMERLKIVAGSKMQGLLE |
| | RVNTEIHFVTKCAFQPPPSCLRFVQTNISRLLQETSEQLVALKPWITRQNFSRCLELQCQPDSSTLPPPWSPRPL |
| | EATAPTAPQPSDKTHTCPPCPAPEAAGGPSVFLFPPKPKDTLMISRTPEVTCVVVDVSHEDPEVKFNWYVD |
| | GVEVHNAKTKPREEQYNSTYRVVSVLTVLHQDWLNGKEYKCQVSNKALPAPIEKTISKAKGQPREPQVYTL |
| | PPCRDELTKNQVSLWCLVKGFYPSDIAVEWESNGQPENNYKTTPPVLDSDGSFFLYSKLTVDKSRWQQG |
| | NVFSCSVMHEALHNHYTQKSLSLSPGKGGSGGSGGSGGSGGSGGSGGSGGSGGSGGSGGSGGSGGS |
| | GGSGGSGGSGGSGGSGGSGGSCDLPQTHSLGSRRTLMLLAQMRKISLFSCLKDRHDFGFPQEEFGNQF |
| | QKAETIPVLHEMIQQIFNLFSTKDSSAAWDETLLDKFYTELYQQLNDLEACVIQGVGVEETPLMKEDSILAVR |
| | KYFQRITLYLKEKKYSPCAWEVVRAEIMASFSLSTNLQESLRSKE |
| 5. | P-2172: FLT3L_27-160-5*GGS-Fc3 |
| | (SEQ ID NO: 50) |
| | TQDCSFQHSPISSDFAVKIRELSDYLLQDYPVTVASNLQDEELCGGLWRLVLAQRWMERLKIVAGSKMQGLLE |
| | RVNTEIHFVTKCAFQPPPSCLRFVQTNISRLLQETSEQLVALKPWITRQNFSRCLELQCQPGGSGGSGGSGGS |
| | GGSDKTHTCPPCPAPEAAGGPSVFLFPPKPKDTLMISRTPEVTCVVVDVSHEDPEVKFNWYVDGVEVHNA |
| | KTKPREEQYNSTYRVVSVLTVLHQDWLNGKEYKCQVSNKALPAPIEKTISKAKGQPREPQVCTLPPSRDEL |
| | TKNQVSLSCAVKGFYPSDIAVEWESNGQPENNYKTTPPVLDSDGSFFLVSKLTVDKSRWQQGNVFSCSV |
| | MHEALHNHYTQKSLSLSPGK |
| 6. | P-2173: FLT3L_27-160-5*GGS-Fc4-AFN |
| | (SEQ ID NO: 51) |
| | TQDCSFQHSPISSDFAVKIRELSDYLLQDYPVIVASNLQDEELCGGLWRLVLAQRWMERLKIVAGSKMQGLLE |
| | RVNTEIHFVTKCAFQPPPSCLRFVQTNISRLLQETSEQLVALKPWITRQNFSRCLELQCQPGGSGGSGGSGGS |
| | GGSDKTHTCPPCPAPEAAGGPSVFLFPPKPKDTLMISRTPEVTCVVVDVSHEDPEVKFNWYVDGVEVHNA |
| | KTKPREEQYNSTYRVVSVLTVLHQDWLNGKEYKCQVSNKALPAPIEKTISKAKGQPREPQVYTLPPCRDEL |
| | TKNQVSLWCLVKGFYPSDIAVEWESNGQPENNYKTTPPVLDSDGSFFLYSKLTVDKSRWQQGNVFSCSV |
| | MHEALHNHYTQKSLSLSPGKGGSGGSGGSGGSGGSGGSGGSGGSGGSGGSGGSGGSGGSGGSGGS |
| | GGSGGSGGSGGSGGSCDLPQTHSLGSRRTLMLLAQMRKISLFSCLKDRHDFGFPQEEFGNQFQKAETIP |
| | VLHEMIQQIFNLFSTKDSSAAWDETLLDKFYTELYQQLNDLEACVIQGVGVEETPLMKEDSILAVRKYFQRIT |
| | LYLKEKKYSPCAWEVVRAEIMASFSLSTNLQESLRSKE |
| 7. | 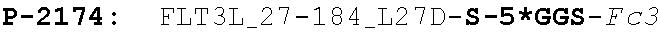 |
| | (SEQ ID NO: 52) |
| | 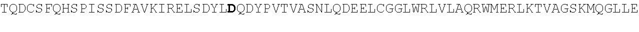 |
| | RVNTEIHFVTKCAFQPPPSCLRFVQTNISRLLQETSEQLVALKPWITRQNFSRCLELQCQPDSSTLPPPWSPRPL |
| | EATAPTAPQPSGGSGGSGGSGGSGGSDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLHQDWLNGKEYKCQVSNKALPAPIEKT |
| | ISKAKGQPREPQVCTLPPSRDELTKNQVSLSCAVKGFYPSDIAVEWESNGQPENNYKTTPPVLDSDGSFFL |
| | VSKLTVDKSRWQQGNVFSCSVMHEALHNHYTQKSLSLSPGK |
| 8. | 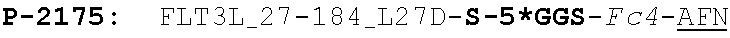 |
| | (SEQ ID NO: 53) |
| | 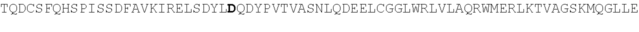 |
| | RVNTEIHFVTKCAFQPPPSCLRFVQTNISRLLQETSEQLVALKPWITRQNFSRCLELQCQPDSSTLPPPWSPRPL |
| | EATAPTAPQPSGGSGGSGGSGGSGGSDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLHQDWLNGKEYKCQVSNKALPAPIEKT |
| | ISKAKGQPREPQVYTLPPCRDELTKNQVSLWCLVKGFYPSDIAVEWESNGQPENNYKTTPPVLDSDGSFFL |
| | YSKLTVDKSRWQQGNVFSCSVMHEALHNHYTQKSLSLSPGKGGSGGSGGSGGSGGSGGSGGSGGSGG |
| | SGGSGGSGGSGGSGGSGGSGGSGGSGGSGGSGGSCDLPQTHSLGSRRTLMLLAQMRKISLFSCLKDR |
| | HDFGFPQEEFGNQFQKAETIPVLHEMIQQIFNLFSTKDSSAAWDETLLDKFYTELYQQLNDLEACVIQGVGV |
| | EETPLMKEDSILAVRKYFQRITLYLKEKKYSPCAWEVVRAEIMASFSLSTNLQESLRSKE |
| 9. | 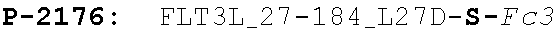 |
| | (SEQ ID NO: 54) |
| | 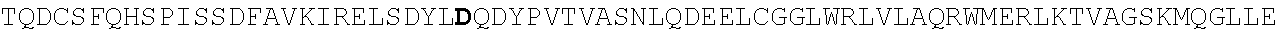 |
| | RVNTEIHFVTKCAFQPPPSCLRFVQTNISRLLQETSEQLVALKPWITRQNFSRCLELQCQPDSSTLPPPWSPRPL |
| | EATAPTAPQPSDKTHTCPPCPAPEAAGGPSVFLFPPKPKDTLMISRTPEVTCVVVDVSHEDPEVKFNWYVD |
| | GVEVHNAKTKPREEQYNSTYRVVSVLTVLHQDWLNGKEYKCQVSNKALPAPIEKTISKAKGQPREPQVCTL |
| | PPSRDELTKNQVSLSCAVKGFYPSDIAVEWESNGQPENNYKTTPPVLDSDGSFFLVSKLTVDKSRWQQGN |
| | VFSCSVMHEALHNHYTQKSLSLSPGK |
| 10. | 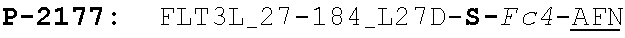 |
| | (SEQ ID NO: 55) |
| | 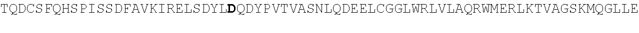 |
| | RVNTEIHFVTKCAFQPPPSCLRFVQTNISRLLQETSEQLVALKPWITRQNFSRCLELQCQPDSSTLPPPWSPRPL |
| | EATAPTAPQPSDKTHTCPPCPAPEAAGGPSVFLFPPKPKDTLMISRTPEVTCVVVDVSHEDPEVKFNWYVD |
| | GVEVHNAKTKPREEQYNSTYRVVSVLTVLHQDWLNGKEYKCQVSNKALPAPIEKTISKAKGQPREPQVYTL |
| | PPCRDELTKNQVSLWCLVKGFYPSDIAVEWESNGQPENNYKTTPPVLDSDGSFFLYSKLTVDKSRWQQG |
| | NVFSCSVMHEALHNHYTQKSLSLSPGKGGSGGSGGSGGSGGSGGSGGSGGSGGSGGSGGSGGSGGS |
| | GGSGGSGGSGGSGGSGGSGGSCDLPQTHSLGSRRTLMLLAQMRKISLFSCLKDRHDFGFPQEEFGNQF |
| | QKAETIPVLHEMIQQIFNLFSTKDSSAAWDETLLDKFYTELYQQLNDLEACVIQGVGVEETPLMKEDSILAVR |
| | KYFQRITLYLKEKKYSPCAWEVVRAEIMASFSLSTNLQESLRSKE |
| 11. | 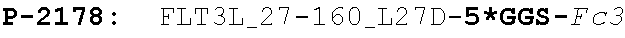 |
| | (SEQ ID NO: 56) |
| | 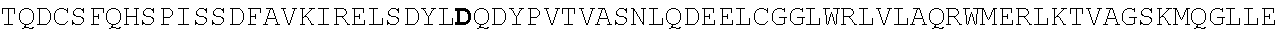 |
| | RVNTEIHFVTKCAFQPPPSCLRFVQTNISRLLQETSEQLVALKPWITRQNFSRCLELQCQPGGSGGSGGSGGS |
| | GGSDKTHTCPPCPAPEAAGGPSVFLFPPKPKDTLMISRTPEVTCVVVDVSHEDPEVKFNWYVDGVEVHNA |
| | KTKPREEQYNSTYRVVSVLTVLHQDWLNGKEYKCQVSNKALPAPIEKTISKAKGQPREPQVCTLPPSRDEL |
| | TKNQVSLSCAVKGFYPSDIAVEWESNGQPENNYKTTPPVLDSDGSFFLVSKLTVDKSRWQQGNVFSCSV |
| | MHEALHNHYTQKSLSLSPGK |
| 12. | 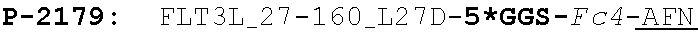 |
| | (SEQ ID NO: 57) |
| | 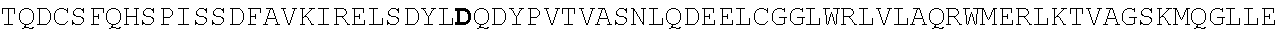 |
| | RVNTEIHFVTKCAFQPPPSCLRFVQTNISRLLQETSEQLVALKPWITRQNFSRCLELQCQPGGSGGSGGSGGS |
| | GGSDKTHTCPPCPAPEAAGGPSVFLFPPKPKDTLMISRTPEVTCVVVDVSHEDPEVKFNWYVDGVEVHNA |
| | KTKPREEQYNSTYRVVSVLTVLHQDWLNGKEYKCQVSNKALPAPIEKTISKAKGQPREPQVYTLPPCRDEL |
| | TKNQVSLWCLVKGFYPSDIAVEWESNGQPENNYKTTPPVLDSDGSFFLYSKLTVDKSRWQQGNVFSCSV |
| | MHEALHNHYTQKSLSLSPGKGGSGGSGGSGGSGGSGGSGGSGGSGGSGGSGGSGGSGGSGGSGGS |
| | GGSGGSGGSGGSGGSCDLPQTHSLGSRRTLMLLAQMRKISLFSCLKDRHDFGFPQEEFGNQFQKAETIP |
| | VLHEMIQQIFNLFSTKDSSAAWDETLLDKFYTELYQQLNDLEACVIQGVGVEETPLMKEDSILAVRKYFQRIT |
| | LYLKEKKYSPCAWEWRAEIMASFSLSTNLQESLRSKE |

### Example 3: Comparison Split Vs Single Chain Formats for FLT3L Fc AFN Variants

In this example, we evaluated the potential of the Fc formatted IFN based AFNs targeted by FLT3L aiming to selectively activate FLT3 positive cells using constructs in which two copies of FLT3L are presented on one single Fc-chain. The possibilities for such single chain FLT3L Fc constructs in a ‘knob-in-hole’ Fc context are show in FIGS. 24A-H and FIGS. 25A-L. For the exemplified heterodimer (see for structure FIG. 24A) the single chain FLT3L sequence (based on Lu et al. 2002 Acta Biochimica Et Biophysica Sinica 2002, 34(6): 697-702) was, via the flexible 10*GGS-linker and in the pcDNA 3.4 expression vector, fused to the human IgG1 Fc sequence containing the L234A_L235A_K322Q effector mutations and the ‘hole’ modifications Y349C_T366S_L368A_Y407V. Second AFN partner, also cloned in the pcDNA 3.4 vector, consists of the fusion between the human IgG1 Fc sequence containing the L234A_L235A_K322Q effector mutations and the ‘knob’ modifications S354C_T366W and the hIFNa2 sequence with the AFN mutation R149A and deletion of the O-glycosylation site at T106 by mutation to E (see sequences below).

Single chain FLT3L AFN was produced, purified and biological activity measured as described in Example 2. Data in FIGS. 26A-B clearly illustrates that single chain FLT3L targeted AFN is only active on FLT3 expressing cells. Even at the highest concentration, no luciferase induction could be observed. Of note, both cell-lines are comparable sensitive to wild type IFN.

### Sequences:

1. P-2180: scFlt3L-Fc3
(SEQ ID NO: 58)
TQDCSFQHSPISSDFAVKIRELSDYLLQDYPVIVASNLQDEELCGGLWR
LVLAQRWMERLKIVAGSKMQGLLERVNTEIHFVTKCAFQPPPSCLRFVQ
TNISRLLQETSEQLVALKPWITRQNFSRCLELQCQPDSSTLPPPWSPRP
LEASGGGGSGGGGSGGGGSTQDCSFQHSPISSDFAVKIRELSDYLLQDY
PVIVASNLQDEELCGGLWRLVLAQRWMERLKTVAGSKMQGLLERVNTEI
HFVTKCAFQPPPSCLRFVQTNISRLLQETSEQLVALKPWITRQNFSRCL
ELQCQPDSSTLPPPWSPRPLEATAPTAPGGSGGSGGSGGSGGSGGSGGS
GGSGGSGGSDKTHTCPPCPAPEAAGGPSVFLFPPKPKDTLMISRTPEVT
CVVVDVSHEDPEVKFNWYVDGVEVHNAKTKPREEQYNSTYRVVSVLTVL
HQDWLNGKEYKCQVSNKALPAPIEKTISKAKGQPREPQVCTLPPSRDEL
TKNQVSLSCAVKGFYPSDIAVEWESNGQPENNYKTTPPVLDSDGSFFLV
SKLTVDKSRWQQGNVFSCSVMHEALHNHYTQKSLSLSPGK
2. P-1414: Fc4-AFN
(SEQ ID NO: 59)
DKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKE
YKCQVSNKALPAPIEKTISKAKGQPREPQVYTLPPCRDELTKNQVSLWC
LVKGFYPSDIAVEWESNGQPENNYKTTPPVLDSDGSFFLYSKLTVDKSR
WQQGNVFSCSVMHEALHNHYTQKSLSLSPGKGGSGGSGGSGGSGGSGGS
GGSGGSGGSGGSGGSGGSGGSGGSGGSGGSGGSGGSGGSGGSCDLPQTH
SLGSRRTLMLLAQMRKISLFSCLKDRHDFGFPQEEFGNQFQKAETIPVL
HEMIQQIFNLFSTKDSSAAWDETLLDKFYTELYQQLNDLEACVIQGVGV
EETPLMKEDSILAVRKYFQRITLYLKEKKYSPCAWEVVRAEIMASFSLS
TNLQESLRSKE

### Example 4: Manufacturability of Split and Single Chain FLT3L AFNs

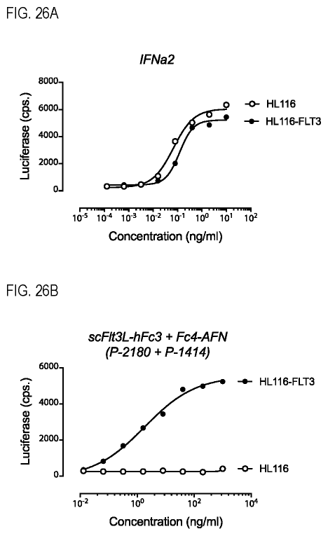

In this example, we compared the manufacturability of FLT3L AFNs in the split or single chain Fc format. For the split format, we chose the variants in which FLT3L aa 27-184 sequence is fused to the Fc arms via a 5*GGS linker (P-2168+P-2169) and the L27D mutant thereof (P-2174+P-2175; for sequences see example 2). For single chain FLT3L AFN (P-2180+P-1414; sequences see example 3). FLT3L AFNs were produced in ExpiCHO cells (ThermoFisher) according to the manufacturer's instructions. Seven days post transfection, supernatant was harvested and cells removed by centrifugation. Proteins were purified on a 5 ml Protein A column (GE Healthcare) on an ÄKTA pure instrument (GE Healthcare). Purified proteins were further analyzed using size exclusion chromatography on the ÄKTA on a Superdex 200 Increase 10/300 column (GE Healthcare). SEC profiles and the subsequent analysis by SDS-PAGE of each peak in FIGS. 27A-C illustrate that (i) the peak eluting at the 158 kD marker is mainly a dimeric P-1414 form (only observed for the P-2180+P-1414 combination); (ii) the peak eluting left of 158 kD marker is identified as the desired product found with the highest abundancy for P-2180+P-1414 (78% versus 57% for P-2168+P-2169, and 25% for P-2174+P-2175); (iii) in the split chain production with the L27D mutation (P-2174+P-2175) a dominant single higher molecular weight species is seen while for the other split chain construct there is a high tendency to form even multiple high molecular weight species (P-2168+P-2169). The HL116 reporter assay in parental HL116 vs HL116-FLT3 cells confirmed for P-2180+P-1414 that the peak left of the 158 kD marker is indeed the correct material as it is about 25 times more active on HL116-FLT3 cells active compared to the peak at 158 kD.

### Example 5: Single Chain FLT3L Fc AFN Variants

In this example, the expression and manufacturability of all of the constructs listed in “Sequences” below was undertaken. These single Chain FLT3L Fc AFN variants essentially differ in the length of the FLT3L sequence used (aa 27-182, aa 27-177 and aa 27-160) and the presence or absence of the L27D mutation in the FLT3L dimerization interface. Two such FLT3L sequences will be fused by a 3*GGGGS linker. Resulting FLT3L sequences were, via the flexible 10*GGS-linker and in the pcDNA 3.4 expression vector, fused to the human IgG1 Fc sequence containing the L234A_L235A_K322Q effector mutations and the ‘hole’ modifications Y349C_T366S_L368A_Y407V. The second AFN partner, also cloned in the pcDNA 3.4 vector, consists of the fusion between the human IgG1 Fc sequence containing the L234A_L235A_K322Q effector mutations and the ‘knob’ modifications S354C_T366W and the hIFNa2 sequence with the AFN mutation R149A and deletion of the O-glycosylation site at T106 by mutation to E. Constructs were expressed in ExpiCHO cells (ThermoFisher) according to the manufacturer's instructions. Seven days post transfection, recombinant proteins were purified using protein A spin plates (ThermoFisher), quantified and assessed for purity using SDS-PAGE.

Biological activity of resulting proteins was measured, in 2 independent experiments, on parental HL116 and HL116-FLT3 cells as described in Example 2. Data in Table 6 shows that (i) all tested FLT3L AFN variants induce IFNAR dependent signaling in HL116-FLT3 cells, (ii) no such signaling detectable at all on the parental HL116 cell-line even at the highest concentration tested and thus all these variants have a very high selectivity window which is completely absent for wild type IFNα2, (iii) FLT3L variants aa 27-182 and aa 27-177 have comparable activity while even the FLT3L variants based on aa 27-160 are still remarkably potent (iv) the L27D mutation tends to decrease signaling capacities somewhat in all three length-variants which paves the way as a solution to optimize manufacturability.

TABLE 6
EC50 of IFNAR signaling in HL116 cells expressed in ng/ml. If the reporter activity at the highest
concentration was detectable but did not reach EC50 levels this is indicated with “>”. In case
at the highest concentration no reporter activity was detected this is indicated by “>>”.

| | Experiment 1 | | | Experiment 2 | | |
|---|---|---|---|---|---|---|
| | | HL116- | | | HL116- | |
| EC50 ng/ml | HL116 | FLT3 | ratio | HL116 | FLT3 | ratio |
| IFNa2 | 0.12 | 0.13 | 1 | 0.22 | 0.17 | 0.8 |
| P-2410 + P-1414 | | >>1000 | | 2.2 | >>200 | 90 |
| P-2411 + P-1414 | 6.1 | >>1000 | >>160 | 11.4 | >>1000 | >>85 |
| P-2412 + P-1414 | 2.1 | >>1000 | >>475 | | >>1000 | |
| P-2413 + P-1414 | 13.6 | >>1000 | >>70 | 18.4 | >>1000 | >>50 |
| P-2414 + P-1414 | 22.0 | >>1000 | >>45 | 16.6 | >>1000 | >>60 |
| P-2415 + P-1414 | 44.3 | >>1000 | >>20 | 64.0 | >>1000 | >>15 |

### Sequences:

| | |
|---|---|
| 1. | P-2410: FLT3L_27-182-3*GGGGS-FLT3L_27-182-10*GGS-Fc3 |
| | (SEQ ID NO: 60) |
| | TQDCSFQHSPISSDFAVKIRELSDYLLQDYPVTVASNLQDEELCGGLWRLVLAQRWMERLKTVAGSKMQG |
| | LLERVNTEIHFVTKCAFQPPPSCLRFVQTNISRLLQETSEQLVALKPWITRQNFSRCLELQCQPDSSTLPPP |
| | WSPRPLEATAPTAPGGGGSGGGGSGGGGSTQDCSFQHSPISSDFAVKIRELSDYLLQDYPVTVASNLQDE |
| | ELCGGLWRLVLAQRWMERLKTVAGSKMQGLLERVNTEIHFVTKCAFQPPPSCLRFVQTNISRLLQETSEQL |
| | VALKPWITRQNFSRCLELQCQPDSSTLPPPWSPRPLEATAPTAPGGSGGSGGSGGSGGSGGSGGSGGS |
| | GGSGGSDKTHTCPPCPAPEAAGGPSVFLFPPKPKDTLMISRTPEVTCVVVDVSHEDPEVKFNWYVDGVEV |
| | HNAKTKPREEQYNSTYRVVSVLTVLHQDWLNGKEYKCQVSNKALPAPIEKTISKAKGQPREPQVCTLPPSR |
| | DELTKNQVSLSCAVKGFYPSDIAVEWESNGQPENNYKTTPPVLDSDGSFFLVSKLTVDKSRWQQGNVFSC |
| | SVMHEALHNHYTQKSLSLSPGK |
| 2. | 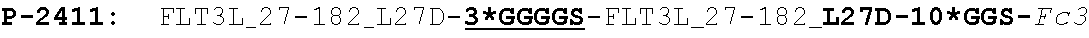 |
| | (SEQ ID NO: 61) |
| | 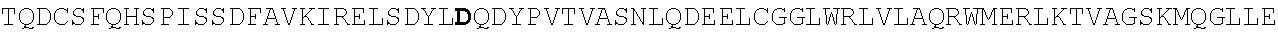 |
| | LLERVNTEIHFVTKCAFQPPPSCLRFVQTNISRLLQETSEQLVALKPWITRQNFSRCLELQCQPDSSTLPPP |
| | 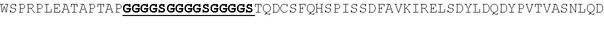 |
| | EELCGGLWRLVLAQRWMERLKTVAGSKMQGLLERVNTEIHFVTKCAFQPPPSCLRFVQTNISRLLQETSEQ |
| | LVALKPWITRQNFSRCLELQCQPDSSTLPPPWSPRPLEATAPTAPGGSGGSGGSGGSGGSGGSGGSGG |
| | SGGSGGSDKTHTCPPCPAPEAAGGPSVFLFPPKPKDTLMISRTPEVTCVVVDVSHEDPEVKFNWYVDGVE |
| | VHNAKTKPREEQYNSTYRVVSVLTVLHQDWLNGKEYKCQVSNKALPAPIEKTISKAKGQPREPQVCTLPPS |
| | RDELTKNQVSLSCAVKGFYPSDIAVEWESNGQPENNYKTTPPVLDSDGSFFLVSKLTVDKSRWQQGNVFS |
| | CSVMHEALHNHYTQKSLSLSPGK |
| 3. | P-2412: FLT3L_27-177-3*GGGGS-FLT3L_27-177-10*GGS-Fc3 |
| | (SEQ ID NO: 62) |
| | TQDCSFQHSPISSDFAVKIRELSDYLLQDYPVTVASNLQDEELCGGLWRLVLAQRWMERLKTVAGSKMQG |
| | LLERVNTEIHFVTKCAFQPPPSCLRFVQTNISRLLQETSEQLVALKPWITRQNFSRCLELQCQPDSSTLPPP |
| | WSPRPLEATGGGGSGGGGSGGGGSTQDCSFQHSPISSDFAVKIRELSDYLLQDYPVTVASNLQDEELCG |
| | GLWRLVLAQRWMERLKTVAGSKMQGLLERVNTEIHFVTKCAFQPPPSCLRFVQTNISRLLQETSEQLVALK |
| | PWITRQNFSRCLELQCQPDSSTLPPPWSPRPLEATGGSGGSGGSGGSGGSGGSGGSGGSGGSGGSDK |
| | THTCPPCPAPEAAGGPSVFLFPPKPKDTLMISRTPEVTCVVVDVSHEDPEVKFNWYVDGVEVHNAKTKPR |
| | EEQYNSTYRVVSVLTVLHQDWLNGKEYKCQVSNKALPAPIEKTISKAKGQPREPQVCTLPPSRDELTKNQV |
| | SLSCAVKGFYPSDIAVEWESNGQPENNYKTTPPVLDSDGSFFLVSKLTVDKSRWQQGNVFSCSVMHEALH |
| | NHYTQKSLSLSPGK |
| 4. | 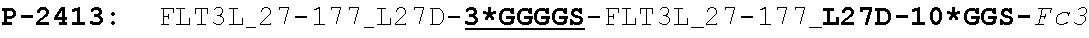 |
| | (SEQ ID NO: 63) |
| | 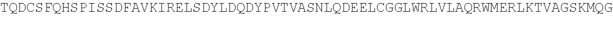 |
| | LLERVNTEIHFVTKCAFQPPPSCLRFVQTNISRLLQETSEQLVALKPWITRQNFSRCLELQCQPDSSTLPPP |
| | 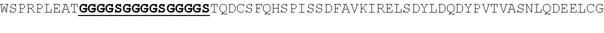 |
| | GLWRLVLAQRWMERLKTVAGSKMQGLLERVNTEIHFVTKCAFQPPPSCLRFVQTNISRLLQETSEQLVALK |
| | PWITRQNFSRCLELQCQPDSSTLPPPWSPRPLEATGGSGGSGGSGGSGGSGGSGGSGGSGGSGGSDK |
| | THTCPPCPAPEAAGGPSVFLFPPKPKDTLMISRTPEVTCVVVDVSHEDPEVKFNWYVDGVEVHNAKTKPR |
| | EEQYNSTYRVVSVLTVLHQDWLNGKEYKCQVSNKALPAPIEKTISKAKGQPREPQVCTLPPSRDELTKNQV |
| | SLSCAVKGFYPSDIAVEWESNGQPENNYKTTPPVLDSDGSFFLVSKLTVDKSRWQQGNVFSCSVMHEALH |
| | NHYTQKSLSLSPGK |
| 5. | P-2414: FLT3L_27-160-3*GGGGS-FLT3L_27-160-10*GGS-Fc3 |
| | (SEQ ID NO: 64) |
| | TQDCSFQHSPISSDFAVKIRELSDYLLQDYPVTVASNLQDEELCGGLWRLVLAQRWMERLKTVAGSKMQG |
| | LLERVNTEIHFVTKCAFQPPPSCLRFVQTNISRLLQETSEQLVALKPWITRQNFSRCLELQCQPGGGGSGG |
| | GGSGGGGSTQDCSFQHSPISSDFAVKIRELSDYLLQDYPVTVASNLQDEELCGGLWRLVLAQRWMERLKT |
| | VAGSKMQGLLERVNTEIHFVTKCAFQPPPSCLRFVQTNISRLLQETSEQLVALKPWITRQNFSRCLELQCQP |
| | GGSGGSGGSGGSGGSGGSGGSGGSGGSGGSDKTHTCPPCPAPEAAGGPSVFLFPPKPKDTLMISRTPE |
| | VTCVVVDVSHEDPEVKFNWYVDGVEVHNAKTKPREEQYNSTYRVVSVLTVLHQDWLNGKEYKCQVSNKA |
| | LPAPIEKTISKAKGQPREPQVCTLPPSRDELTKNQVSLSCAVKGFYPSDIAVEWESNGQPENNYKTTPPVLD |
| | SDGSFFLVSKLTVDKSRWQQGNVFSCSVMHEALHNHYTQKSLSLSPGK |
| 6. | 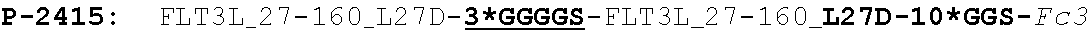 |
| | (SEQ ID NO: 65) |
| | 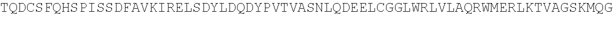 |
| | LLERVNTEIHFVTKCAFQPPPSCLRFVQTNISRLLQETSEQLVALKPWITRQNFSRCLELQCQPGGGGSGG |
| | 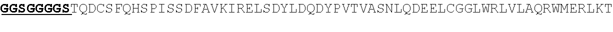 |
| | VAGSKMQGLLERVNTEIHFVTKCAFQPPPSCLRFVQTNISRLLQETSEQLVALKPWITRQNFSRCLELQCQP |
| | GGSGGSGGSGGSGGSGGSGGSGGSGGSGGSDKTHTCPPCPAPEAAGGPSVFLFPPKPKDTLMISRTPE |
| | VTCVVVDVSHEDPEVKFNWYVDGVEVHNAKTKPREEQYNSTYRVVSVLTVLHQDWLNGKEYKCQVSNKA |
| | LPAPIEKTISKAKGQPREPQVCTLPPSRDELTKNQVSLSCAVKGFYPSDIAVEWESNGQPENNYKTTPPVLD |
| | SDGSFFLVSKLTVDKSRWQQGNVFSCSVMHEALHNHYTQKSLSLSPGK |
| 7. | P-1414: Fc4-AFN |
| | (SEQ ID NO: 66) |
| | DKTHTCPPCPAPEAAGGPSVFLFPPKPKDTLMISRTPEVTCVVVDVSHEDPEVKFNWYVDGVEVHNAKTK |
| | PREEQYNSTYRVVSVLTVLHQDWLNGKEYKCQVSNKALPAPIEKTISKAKGQPREPQVYTLPPCRDELTKN |
| | QVSLWCLVKGFYPSDIAVEWESNGQPENNYKTTPPVLDSDGSFFLYSKLTVDKSRWQQGNVFSCSVMHE |
| | ALHNHYTQKSLSLSPGKGGSGGSGGSGGSGGSGGSGGSGGSGGSGGSGGSGGSGGSGGSGGSGGS |
| | GGSGGSGGSGGSCDLPQTHSLGSRRTLMLLAQMRKISLFSCLKDRHDFGFPQEEFGNQFQKAETIPVLHE |
| | MIQQIFNLFSTKDSSAAWDETLLDKFYTELYQQLNDLEACVIQGVGVEETPLMKEDSILAVRKYFQRITLYLK |
| | EKKYSPCAWEVVRAEIMASFSLSTNLQESLRSKE |

### Example 6: Single Chain FLT3L AFN Variants without Fc Domain

In this example, we will evaluate the expression, manufacturability and biological activity of different single chain FLT3L AFNs without Fc domain and compare with single copy FLT3L AFN (P-2373). The different variants essentially differ in the length of the FLT3L sequence used (aa 27-182, aa 27-177 and aa 27-160) and the presence or absence of the L27D mutation in the FLT3L dimerization interface. Two FLT3L sequences will be fused by a 3*GGGGS linker and the resulting FLT3L single chain will be, via the flexible 10*GGS-linker fused the AFN partner consisting of the hIFNa2 sequence with the AFN mutation R149A and deletion of the O-glycosylation site at T106 by mutation to E. Constructs are cloned in the pcDNA 3.4 vector with a C-terminal histidine tag and expressed in ExpiCHO and purified by metal-affinity.

### Sequences:

| | |
|---|---|
| 1. | FLT3L_27-182-3*GGGGS-FLT3L_27-182-10*GGS-AFN-H6 |
| | (SEQ ID NO: 67) |
| | TQDCSFQHSPISSDFAVKIRELSDYLLQDYPVTVASNLQDEELCGGLWRLVLAQRWMERLKTVAGSKMQG |
| | LLERVNTEIHFVTKCAFQPPPSCLRFVQTNISRLLQETSEQLVALKPWITRQNFSRCLELQCQPDSSTLPPP |
| | WSPRPLEATAPTAPGGGGSGGGGSGGGGSTQDCSFQHSPISSDFAVKIRELSDYLLQDYPVTVASNLQDE |
| | ELCGGLWRLVLAQRWMERLKTVAGSKMQGLLERVNTEIHFVTKCAFQPPPSCLRFVQTNISRLLQETSEQL |
| | VALKPWITRQNFSRCLELQCQPDSSTLPPPWSPRPLEATAPTAPGGSGGSGGSGGSGGSGGSGGSGGS |
| | GGSGGSCDLPOTHSLGSRRTLMLLAQMRKISLFSCLKDRHDFGFPQEEFGNQFQKAETIPVLHEMIQQIFN |
| | LFSTKDSSAAWDETLLDKFYTELYQQLNDLEACVIQGVGVEETPLMKEDSILAVRKYFQRITLYLKEKKYSPC |
| | AWEVVRAEIMASFSLSTNLQESLRSKE |
| 2. | 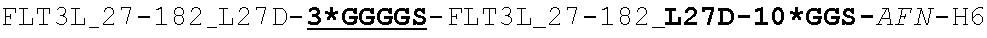 |
| | (SEQ ID NO: 68) |
| | 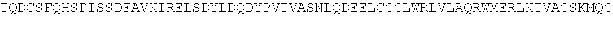 |
| | LLERVNTEIHFVTKCAFQPPPSCLRFVQTNISRLLQETSEQLVALKPWITRQNFSRCLELQCQPDSSTLPPP |
| | 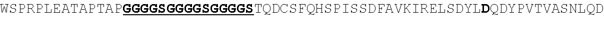 |
| | EELCGGLWRLVLAQRWMERLKTVAGSKMQGLLERVNTEIHFVTKCAFQPPPSCLRFVQTNISRLLQETSE |
| | QLVALKPWITRQNFSRCLELQCQPDSSTLPPPWSPRPLEATAPTAPGGSGGSGGSGGSGGSGGSGGSG |
| | GSGGSGGSCDLPQTHSLGSRRTLMLLAQMRKISLFSCLKDRHDFGFPQEEFGNQFQKAETIPVLHEMIQQI |
| | FNLFSTKDSSAAWDETLLDKFYTELYQQLNDLEACVIQGVGVEETPLMKEDSILAVRKYFQRITLYLKEKKYS |
| | PCAWEVVRAEIMASFSLSTNLQESLRSKE |
| 3. | FLT3L_27-177-3*GGGGS-FLT3L_27-177-10*GGS-AFN-H6 |
| | (SEQ ID NO: 69) |
| | TQDCSFQHSPISSDFAVKIRELSDYLLQDYPVTVASNLQDEELCGGLWRLVLAQRWMERLKTVAGSKMQG |
| | LLERVNTEIHFVTKCAFQPPPSCLRFVQTNISRLLQETSEQLVALKPWITRQNFSRCLELQCQPDSSTLPPP |
| | WSPRPLEATGGGGSGGGGSGGGGSTQDCSFQHSPISSDFAVKIRELSDYLLQDYPVTVASNLQDEELCG |
| | GLWRLVLAQRWMERLKTVAGSKMQGLLERVNTEIHFVTKCAFQPPPSCLRFVQTNISRLLQETSEQLVALK |
| | PWITRQNFSRCLELQCQPDSSTLPPPWSPRPLEATGGSGGSGGSGGSGGSGGSGGSGGSGGSGGSCD |
| | LPQTHSLGSRRTLMLLAQMRKISLFSCLKDRHDFGFPQEEFGNQFQKAETIPVLHEMIQQIFNLFSTKDSSA |
| | AWDETLLDKFYTELYQQLNDLEACVIQGVGVEETPLMKEDSILAVRKYFQRITLYLKEKKYSPCAWEVVRAE |
| | IMASFSLSTNLQESLRSKE |
| 4. | 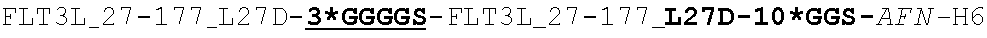 |
| | (SEQ ID NO: 70) |
| | 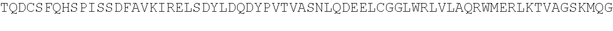 |
| | LLERVNTEIHFVTKCAFQPPPSCLRFVQTNISRLLQETSEQLVALKPWITRQNFSRCLELQCQPDSSTLPPP |
| | 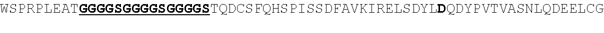 |
| | GLWRLVLAQRWMERLKTVAGSKMQGLLERVNTEIHFVTKCAFQPPPSCLRFVQTNISRLLQETSEQLVALK |
| | PWITRQNFSRCLELQCQPDSSTLPPPWSPRPLEATGGSGGSGGSGGSGGSGGSGGSGGSGGSGGSCD |
| | LPQTHSLGSRRTLMLLAQMRKISLFSCLKDRHDFGFPQEEFGNQFQKAETIPVLHEMIQQIFNLFSTKDSSA |
| | AWDETLLDKFYTELYQQLNDLEACVIQGVGVEETPLMKEDSILAVRKYFQRITLYLKEKKYSPCAWEVVRAE |
| | IMASFSLSTNLQESLRSKE |
| 5. | FLT3L_27-160-3*GGGGS-FLT3L_27-160-10*GGS-AFN-H6 |
| | (SEQ ID NO: 71) |
| | TQDCSFQHSPISSDFAVKIRELSDYLLQDYPVTVASNLQDEELCGGLWRLVLAQRWMERLKTVAGSKMQG |
| | LLERVNTEIHFVTKCAFQPPPSCLRFVQTNISRLLQETSEQLVALKPWITRQNFSRCLELQCQPGGGGSGG |
| | GGSGGGGSTQDCSFQHSPISSDFAVKIRELSDYLLQDYPVTVASNLQDEELCGGLWRLVLAQRWMERLKT |
| | VAGSKMQGLLERVNTEIHFVTKCAFQPPPSCLRFVQTNISRLLQETSEQLVALKPWITRQNFSRCLELQCQP |
| | GGSGGSGGSGGSGGSGGSGGSGGSGGSGGSCDLPQTHSLGSRRTLMLLAQMRKISLFSCLKDRHDFGF |
| | PQEEFGNQFQKAETIPVLHEMIQQIFNLFSTKDSSAAWDETLLDKFYTELYQQLNDLEACVIQGVGVEETPL |
| | MKEDSILAVRKYFQRITLYLKEKKYSPCAWEVVRAEIMASFSLSTNLQESLRSKE |
| 6. | 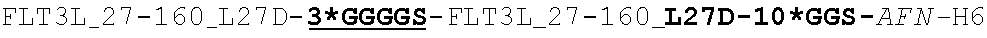 |
| | (SEQ ID NO: 72) |
| | 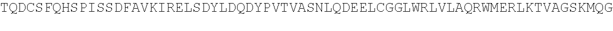 |
| | LLERVNTEIHFVTKCAFQPPPSCLRFVQTNISRLLQETSEQLVALKPWITRQNFSRCLELQCQPGGGGSGG |
| | 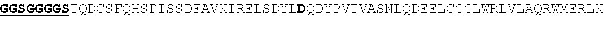 |
| | TVAGSKMQGLLERVNTEIHFVTKCAFQPPPSCLRFVQTNISRLLQETSEQLVALKPWITRQNFSRCLELQCQ |
| | PGGSGGSGGSGGSGGSGGSGGSGGSGGSGGSCDLPQTHSLGSRRTLMLLAQMRKISLFSCLKDRHDFG |
| | FPQEEFGNQFQKAETIPVLHEMIQQIFNLFSTKDSSAAWDETLLDKFYTELYQQLNDLEACVIQGVGVEETP |
| | LMKEDSILAVRKYFQPITLYLKEKKYSPCAWEVVRAEIMASFSLSTNLQESLRSKE |
| 7. | P-2373: FLT3L_27-181-VD-20*GGS-AAAM-AFN-LE-9*His |
| | (SEQ ID NO: 73) |
| | TQDCSFQHSPISSDFAVKIRELSDYLLQDYPVTVASNLQDEELCGGLWRLVLAQRWMERLKTVAGSKMQG |
| | LLERVNTEIHFVTKCAFQPPPSCLRFVQTNISRLLQETSEQLVALKPWITRQNFSRCLELQCQPDSSTLPPP |
| | WSPRPLEATAPTAVDGGSGGSGGSGGSGGSGGSRSGGSGGSGGSGGSGGSGGSGGSGGSGGSGGS |
| | GGSGGSGGSAAAMCDLPQTHSLGSRRTLMLLAQMRRISLFSCLKDRHDFGFPQEEFGNQFQKAETIPVLH |
| | EMIQQIFNLFSTKDSSAAWDETLLDKFYTELYQQLNDLEACVIQGVGVTETPLMKEDSILAVRKYFQRITLYL |
| | KEKKYSPCAWEVVRAEIMASFSLSTNLQESLRSKE |

The construct of SEQ ID NO: 73 was expressed and purified. FIG. 30 shows size exclusion chromatography (SEC) profiles of the purified construct of SEQ ID NO: 73—a monomeric form of Flt3L linked to interferon via a flexible linker. Purified FLT3L-AFN (dark line) with protein markers shown in grey line. The SEC profile of the peak fraction illustrate that the protein behaves in SEC as an about 150 kD protein (i.e. eluting at the 158 kD marker). These data support the formation of non-covalently linked dimer. Such dimers have not been seen with VHH based AFNs and thus are the consequence of an FLT3L dimerization as required for FLT3L receptor binding and signaling. Compare to the size exclusion chromatography (SEC) profiles of FIGS. 27A-C.

### Example 7: Use of FLT3 Targeted AFN for Treatment

In order to assess the anti-tumoral activity of an FLT3 targeted AFN the two following single chain Flt3L chimeric protein constructs were expressed in ExpiCHO and purified by protein A followed by size exclusion chromatography:
scFLT3-Fc-AFN: P-2180+P-1414
scFLT3-Fc: P-2180+P-2038, with P-2038 coding for the Fc4 protein without AFN moiety.

The purified proteins were subsequently evaluated in a tumor model in a humanized mouse. In brief, newborn NSG mice (1-2 days of age) were sublethal irradiated with 100 cGy prior to intrahepatic delivery of 1×10⁵ CD34+ human stem cells (from HLA-A2 positive cord bloods). At week 13 after stem cell transfer mice were subcutaneously inoculated with 25×10⁵ human RL follicular lymphoma cells (ATCC CRL-2261; not sensitive to the direct anti-proliferative effect of IFN). Intravenous treatment with equimolar doses of scFlt3L-Fc fusion (19.8 μg) and scFlt3L-Fc-AFN (25 μg) or buffer were performed at day 12 and 20 after tumor inoculation (n=3 to 5 mice per group). Tumor size (caliper measurements) and body weight were assessed every 2nd or 3rd day.

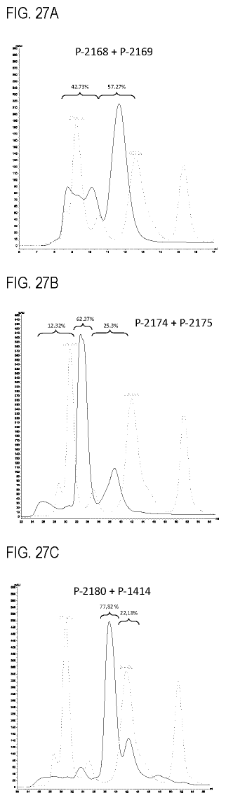

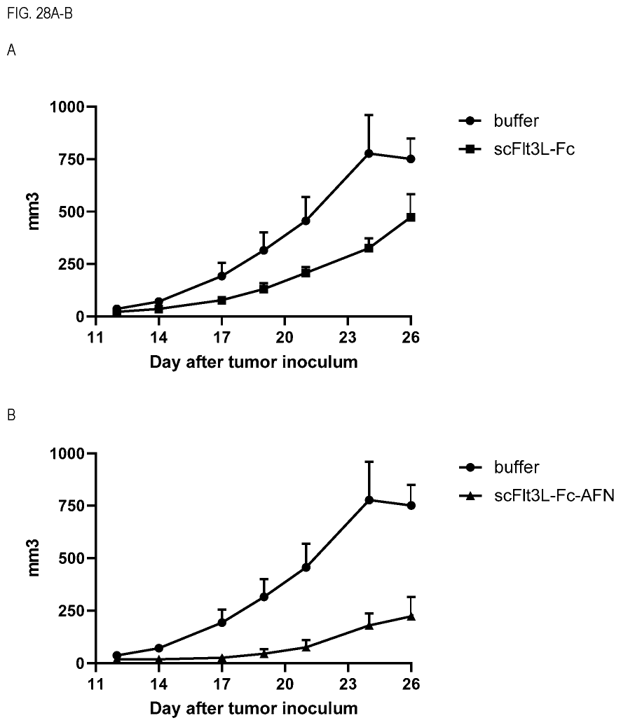

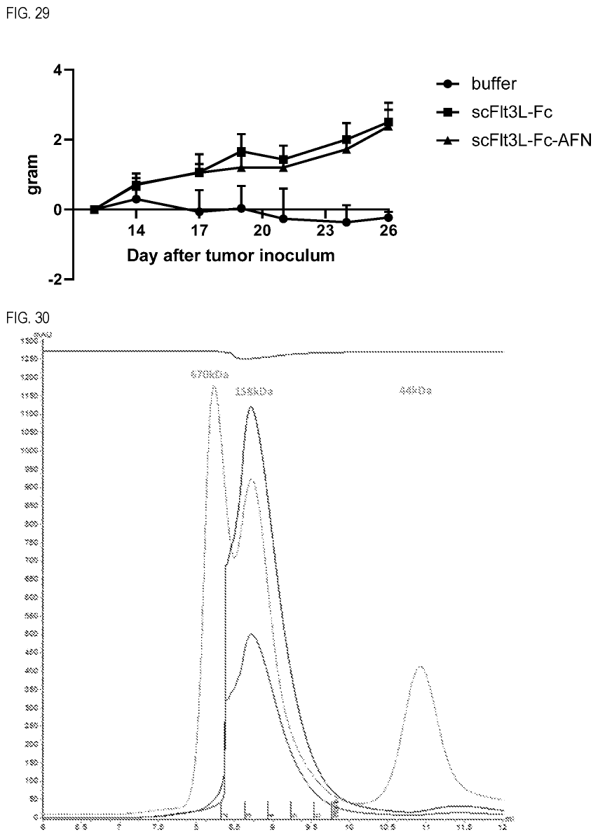

Data in FIG. 28A show the tumor growth until 6 days after the second treatment and demonstrate that the scFlt3L-Fc fusion is functional as tumor growth is reduced as compared to buffer treatment. Data in FIG. 28B demonstrate the potency of the scFlt3L-Fc-AFN protein to even more strongly suppress tumor growth. Body weight evolution, FIG. 29, showed that both group of mice treated with the Flt3L constructs gained weight equally in contrast to buffer treated animals that lost weight.

### Example 8: Generation, Production, Purification and Characterization of Further Flt3L/IFNα1 AFNs

In this example, we evaluated the activity of IFNα1 fused to FLT3L.

The purified protein was evaluated in a tumor model in a humanized mouse. In brief, newborn NSG mice (1-2 days of age) were sublethal irradiated with 100 cGy prior to intrahepatic delivery of 1×10⁵CD34+ human stem cells (from HLA-A2 positive cord bloods). At week 13 after stem cell transfer mice were subcutaneously inoculated with 25×10⁵ human RL follicular lymphoma cells (ATCC CRL-2261; not sensitive to the direct anti-proliferative effect of IFN). Mice were treated daily intraperitoneally with 30 μg of human Flt3L protein, from day 8 to day 18 after tumor inoculation. Daily perilesional injection with buffer or Flt3L-IFNα1 (30 μg) was initiated at day 10 after tumor inoculation, when a palpable tumor was visible (n=5 or 6 mice per group). Tumor size (caliper measurements), body weight and temperature were assessed daily.

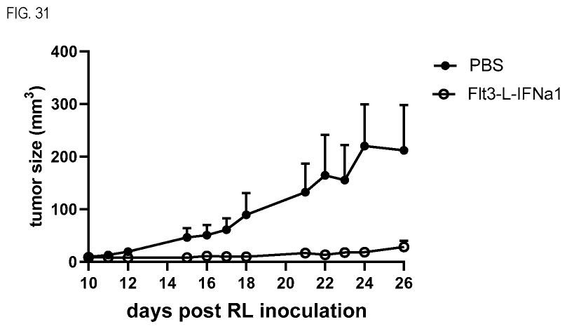

Data in FIG. 31 show the tumor growth until 2 days after the last treatment and demonstrates that Flt3L/IFNα1 strongly suppresses tumor growth. Data on body weight and temperature did not show any major difference between buffer treatment and IFNα1 treatment supporting that the treatment was well tolerated.

What is claimed is:

1. An Fc-based chimeric protein complex comprising:
(i) one or more targeting moieties which specifically bind to an antigen or receptor of interest, the antigen or receptor of interest being FMS-like tyrosine kinase 3 (FLT3);
(ii) an Fc domain, the Fc domain optionally having one or more mutations that reduces or eliminates one or more effector functions of the Fc domain, promotes Fc chain pairing in the Fc domain, and/or stabilizes a hinge region in the Fc domain; and
(iii) a signaling agent or a modified form thereof.

2. The Fc-based chimeric protein complex of claim 1, wherein one targeting moiety is attached to each Fc chain of the Fc domain.

3. The Fc-based chimeric protein complex of claim 1, wherein two targeting moieties are attached to one Fc chain of the Fc domain.

4. The Fc-based chimeric protein complex of claim 3, wherein the two targeting moieties are attached to each other, optionally via a linker.

5. The Fc-based chimeric protein complex of claim 4, wherein the two targeting moieties are attached to the Fc chain, optionally via a linker.

6. The Fc-based chimeric protein complex of any one of claims 1-5, wherein the targeting moiety comprises FLT3L, or a portion thereof.

7. The Fc-based chimeric protein complex of any one of claims 1-6, wherein the targeting moiety comprises the extracellular domain of FLT3L, or a portion thereof.

8. The Fc-based chimeric protein complex of claim 7, wherein the targeting moiety comprises an amino acid sequence having at least 90% identity with any one of SEQ ID NOs: 2-5.

9. The Fc-based chimeric protein complex of claim 8, wherein the targeting moiety comprises an amino acid sequence having at least 95% identity with any one of SEQ ID NOs: 2-5.

10. The Fc-based chimeric protein complex of any one of claims 1-9, wherein the targeting moiety comprises the extracellular domain of FLT3L, or a portion thereof and a mutation that reduces intermolecular extracellular domain dimerization, optionally being a substitution at position L27 of any one of SEQ ID NOs: 2-4, optionally being L27D.

11. The Fc-based chimeric protein complex of any one of claims 1-9, wherein the targeting moiety comprises the extracellular domain of FLT3L, or a portion thereof, and the extracellular domain is a single chain dimer.

12. The Fc-based chimeric protein complex of any one of claims 1-11, wherein the signaling agent is wild type human IFNα2, IFNα1, IFNβ, or IL-13.

13. The Fc-based chimeric protein complex of any one of claims 1-12, wherein the signaling agent comprises an amino acid sequence having at least about 95%, or at least about 97%, or at least about 98% identity with any one of SEQ ID NO: 6, 7, 38, 39, or 74.

14. The Fc-based chimeric protein complex of any one of claims 1-13, wherein the signaling agent is modified to comprise one or more mutations.

15. The Fc-based chimeric protein complex of claim 14, wherein the one or more mutations confer improved safety as compared to a wild type signaling agent.

16. The Fc-based chimeric protein complex of claim 14, wherein the one or more mutations confer reduced affinity for the signaling agent's receptor.

17. The Fc-based chimeric protein complex of claim 14, wherein the one or more mutations confer reduced bioactivity for the signaling agent's receptor.

18. The Fc-based chimeric protein complex of any one of claims 14-17, wherein the one or more mutations allow for attenuation of the signaling agent's activity.

19. The Fc-based chimeric protein complex of claim 18, wherein agonistic or antagonistic activity of the signaling agent is attenuated.

20. The Fc-based chimeric protein complex of any one of claims 15-19, wherein the modified signaling agent comprises one or more mutations which convert its activity from agonistic to antagonistic.

21. The Fc-based chimeric protein complex of any one of claims 15-20, wherein the one or more mutations confer reduced affinity or activity that is restorable by attachment to one or more targeting moiety or upon inclusion in an Fc-based chimeric protein complex.

22. The Fc-based chimeric protein complex of any one of claims 15-21, wherein the one or more mutations confer substantially reduced or ablated affinity or activity that is not substantially restorable by attachment to a targeting moiety or upon inclusion in an Fc-based chimeric protein complex.

23. The Fc-based chimeric protein complex of any one of claims 1-22, wherein the signaling agent is a mutant human IFNα2 comprising an amino acid sequence having at least 95% identity with SEQ ID NO: 6 or 7 and wherein the mutant human IFNα2 has one or more mutations that confer improved safety as compared to a wild type IFNα2 having an amino acid sequence of SEQ ID NO: 6 or 7.

24. The Fc-based chimeric protein complex of claim 23, wherein the human IFNα2 has one or more mutations at positions 144 to 154 with respect to SEQ ID NO: 6 or 7.

25. The Fc-based chimeric protein complex of claim 23, wherein the human IFNα2 has: one or more mutations at positions L15, A19, R22, R23, L26, F27, L30, L30, K31, D32, R33, H34, D35, Q40, H57, E58, Q61, F64, N65, T69, L80, Y85, Y89, D114, L117, R120, R125, K133, K134, R144, A145, M148, R149, S152, L153, and N156 with respect to SEQ ID NO: 6 or 7.

26. The Fc-based chimeric protein complex of claim 25, wherein the mutation is one or more of L15A, A19W, R22A, R23A, L26A, F27A, L30A, L30V, K31A, D32A, R33K, R33A, R33Q, H34A, D35A, Q40A, H57Y, E58N, Q61S, F64A, N65A, T69A, L80A, Y85A, Y89A, D114R, L117A, R120A, R125A, K133A, K134A, R144A, A145G, A145M, M148A, R149A, S152A, L153A, and N156A with respect to SEQ ID NO: 6 or 7.

27. The Fc-based chimeric protein complex of claim 23, wherein the mutant human IFNα2 has one or more mutations at position R33, R144, A145, M148, R149, and L153 with respect to SEQ ID NO: 6 or 7 or wherein the mutant human IFNα2 has one or more mutations selected from R33A, R120E, R144X₁ A145X₂, M148A, R149A, and L153A with respect to amino acid sequence of SEQ ID NO: 6 or 7, wherein X₁ is selected from A, S, T, Y, L, and I, and wherein X₂ is selected from G, H, Y, K, and D.

28. The Fc-based chimeric protein complex of any of claims 13-27, wherein the human IFNα2 has a mutation selected T106A or T106E.

29. The Fc-based chimeric protein complex of any one of claims 1-22, wherein the signaling agent is wild type or a mutant human IFN-α1 which comprises an amino acid sequence having at least about 90%, or at least about 93%, or at least about 95%, or at least about 97%, or at least about 98%, or at least about 99%, or 100% identity with SEQ ID NO: 74, optionally wherein the IFN-α1 comprises:
(i) one or more mutations which confer reduced affinity for interferon-α/β receptor (IFNAR), optionally selected from a substitution at L15, A19, R23, S25, L30, D32, R33, H34, Q40, D115, L118, K121, R126, E133, K134, K135, R145, A146, M149, R150, S153, L154, and N157 or a combination thereof, wherein the positions are in reference to SEQ ID NO: 74, optionally selected from the group consisting of L15A, A19W, R23A, S25A, L30A, L30V, D32A, R33K, R33A, R33Q, H34A, Q40A, D115R, L118A, K121A, K121E, R126A, R126E, E133A, K134A, K135A, R145A, R145D, R145E, R145G, R145H, R145I, R145K, R145L, R145N, R145Q, R145S, R145T, R145V, R145Y, A146D, A146E, A146G, A146H, A146I, A146K, A146L, A146M, A146N, A146Q, A146R, A146S, A146T, A146V, A146Y, M149A, M149V, R150A, S153A, L154A, N157A, L30A-H58Y-E59N-Q62S, R33A-H58Y-E59N-Q625, M149A-H58Y-E59N-Q62S, L154A-H58Y-E59N-Q62S, R145A-H58Y-E59N-Q62S, D115A-R121A, L118A-R121A, L118A-R121A-K122A, R121A-K122A, and R121E-K122E and/or
(ii) one or more mutations which affect aggregation, optionally selected from a substitution or deletion of one or more of position C1, C29, C86, C99, C139 in reference to SEQ ID NO: 74, optionally selected from C86S, C86A, and C86Y.

30. The Fc-based chimeric protein complex of any one of claims 1-22, wherein the signaling agent is a mutant human IFNβ comprising an amino acid sequence having at least 95% identity with SEQ ID NO: 38 and wherein the mutant human IFNβ has one or more mutations that confer improved safety as compared to a wild type IFNβ having an amino acid sequence of SEQ ID NO: 38.

31. The Fc-based chimeric protein complex of claim 30, wherein the mutation is one or more of W22G, R27G, L32A, L32G, R35A, R35G, V148G, L151G, R152A, R152G with respect to amino acid sequence of SEQ ID NO: 38.

32. The Fc-based chimeric protein complex of any one of claims 1-22, wherein the signaling agent is a mutant human IL-1β comprising an amino acid sequence having at least 95% identity with SEQ ID NO: 39 and wherein the mutant human IL-1β has one or more mutations that confer improved safety as compared to a wild type IL-1β having an amino acid sequence of SEQ ID NO: 39.

33. The Fc-based chimeric protein complex of claim 32, wherein the mutation is one or more of A117G/P118G, R120G, R120A, L122A, T125G/L126G, R127G, Q130A, Q130W, Q131G, K132A, S137G/Q138Y, L145G, H146A, H146G, H146E, H146N, H146R, L145A/L147A, Q148E, Q148G, Q148L, Q148G/Q150G, Q150G/D151A, M152G, F162A, F162A/Q164E, F166A, Q164E/E167K, N169G/D170G, 1172A, V174A, K208E, K209A, K209D, K209A/K210A, K219S, K219Q, E221S, E221K, E221S/N224A, N224S/K225S, E244K and N245Q with respect to amino acid sequence of SEQ ID NO: 39.

34. The Fc-based chimeric protein complex of any one of the above claims, wherein the targeting moiety is directed against an immune cell, optionally being a dendritic cell.

35. The Fc-based chimeric protein complex of claim 34, wherein the dendritic cell is a conventional dendritic cell (cDC), optionally being a cDC-1, migratory DC, CDC-2, and Flt3+DC.

36. The Fc-based chimeric protein complex of claim 35, wherein the targeting moiety is directed to a hematopoietic stem cell (HSC), early progenitor cell, immature thymocyte, or steady state dendritic cell (DC).

37. The Fc-based chimeric protein complex of any one of the above claims, wherein the targeting moiety functionally modulates the antigen or receptor of interest.

38. The Fc-based chimeric protein complex of any one of the above claims, wherein the targeting moiety binds but does not functionally modulate the antigen or receptor of interest.

39. The Fc-based chimeric protein complex of any one of the above claims, wherein the targeting moiety directly or indirectly recruits immune cells to tumor cells or to the tumor microenvironment.

40. The Fc-based chimeric protein complex of any one of the above claims, wherein the targeting moiety increases a number of dendritic cells.

41. The Fc-based chimeric protein complex of any one of the above claims, wherein the targeting moiety enhances tumor antigen presentation, optionally by dendritic cells.

42. The Fc-based chimeric protein complex of any one of the above claims, further comprising an additional targeting moiety, optionally being two targeting moieties, which are identical or non-identical.

43. The Fc-based chimeric protein complex of any one of the above claims, comprising an additional signaling agent.

44. The Fc-based chimeric protein complex of any one of the above claims, comprising two signaling agents.

45. The Fc-based chimeric protein complex of any one of the above claims, wherein the Fc-based chimeric protein complex is suitable for use in a patient having one or more of: cancer, infections, immune disorders, autoimmune and/or neurodegenerative disease, cardiovascular diseases, wound, ischemia-related diseases, and/or metabolic diseases.

46. The Fc-based chimeric protein complex of any one of claims 1-45, wherein the Fc domain is from IgG, IgA, IgD, IgM or IgE.

47. The Fc-based chimeric protein complex of claim 46, wherein the IgG is selected from IgG1, IgG2, IgG3, or IgG4.

48. The Fc-based chimeric protein complex of any one of claims 1-45, wherein the Fc domain is from human IgG, IgA, IgD, IgM or IgE.

49. The Fc-based chimeric protein complex of claim 48, wherein the human IgG is selected from human IgG1, IgG2, IgG3, or IgG4.

50. The Fc-based chimeric protein complex of any one of claims 1-49, wherein the Fc chain pairing is promoted by ionic pairing and/or a knob-in-hole pairing.

51. The Fc-based chimeric protein complex of any one of claims 1-50, wherein the one or more mutations to the Fc domain results in an ionic pairing between the Fc chains in the Fc domain.

52. The Fc-based chimeric protein complex of any one of claims 1-51, wherein the one or more mutations to the Fc domain results in a knob-in-hole pairing in the Fc domain.

53. The Fc-based chimeric protein complex of any one of claims 1-52, wherein the one or more mutations to the Fc domain results in the reduction or elimination of the effector function of the Fc domain

54. The Fc-based chimeric protein complex of any one of claims 1-53, wherein the Fc-based chimeric protein complex is a heterodimer and has a trans orientation.

55. The Fc-based chimeric protein complex of any one of claims 1-53, wherein the Fc-based chimeric protein-complex is a heterodimer and has a cis orientation.

56. The Fc-based chimeric protein complex of any one of claims 1-55, wherein the Fc comprises L234A, L235A, and K322Q substitutions in human IgG1 (according to EU numbering).

57. The Fc-based chimeric protein complex of any one of claims 1-52, wherein the Fc is human IgG1, and optionally contains one or more of L234, L235, K322, D265, P329, and P331 (according to EU numbering).

58. The Fc-based chimeric protein complex of any one of claims 1-57, wherein the Fc-based chimeric protein complex has an orientation and/or configuration of any one of FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, 19A-F, 21A-F, 22A-F, 24A-H, and 25A-L.

59. The Fc-based chimeric protein complex of any one of claims 1-58, wherein the Fc-based chimeric protein complex comprises a polypeptide having an amino acid sequence having at least 95%, or at least 98%, or at least 99% identity with any one of SEQ ID NOs: 40, 41, and 46-66.

60. The Fc-based chimeric protein complex of any one of claims 1-58 wherein the Fc-based chimeric protein complex comprises a polypeptide having an amino acid sequence selected from SEQ ID NOs: 40, 41, and 46-66 and less than 10 mutations to the amino acid sequence.

61. The Fc-based chimeric protein complex of claim 60, wherein the Fc-based chimeric protein complex comprises a polypeptide having an amino acid sequence selected from SEQ ID NOs: 40, 41, and 46-66, and less than 5 mutations to the amino acid sequence.

62. The Fc-based chimeric protein complex of claim 60, wherein the Fc-based chimeric protein complex comprises a polypeptide having an amino acid sequence selected from SEQ ID NOs: 40, 41, and 46-66.

63. The Fc-based chimeric protein complex of claim 59, wherein the Fc-based chimeric protein complex comprises a first amino acid sequence having at least 95%, or at least 98%, or at least 99% identity to SEQ ID NO: 40 and a second amino acid sequence having at least 95%, or at least 98%, or at least 99% identity to SEQ ID NO: 41.

64. The Fc-based chimeric protein complex of claim 59, wherein the Fc-based chimeric protein complex comprises a first amino acid sequence having at least 95%, or at least 98%, or at least 99% identity to SEQ ID NO: 46 and a second amino acid sequence having at least 95%, or at least 98%, or at least 99% identity to SEQ ID NO: 47.

65. The Fc-based chimeric protein complex of claim 59, wherein the Fc-based chimeric protein complex comprises a first amino acid sequence having at least 95%, or at least 98%, or at least 99% identity to SEQ ID NO: 48 and a second amino acid sequence having at least 95%, or at least 98%, or at least 99% identity to SEQ ID NO: 49.

66. The Fc-based chimeric protein complex of claim 59, wherein the Fc-based chimeric protein complex comprises a first amino acid sequence having at least 95%, or at least 98%, or at least 99% identity to SEQ ID NO: 50 and a second amino acid sequence having at least 95%, or at least 98%, or at least 99% identity to SEQ ID NO: 51.

67. The Fc-based chimeric protein complex of claim 59, wherein the Fc-based chimeric protein complex comprises a first amino acid sequence having at least 95%, or at least 98%, or at least 99% identity to SEQ ID NO: 52 and a second amino acid sequence having at least 95%, or at least 98%, or at least 99% identity to SEQ ID NO: 53.

68. The Fc-based chimeric protein complex of claim 59, wherein the Fc-based chimeric protein complex comprises a first amino acid sequence having at least 95%, or at least 98%, or at least 99% identity to SEQ ID NO: 54 and a second amino acid sequence having at least 95%, or at least 98%, or at least 99% identity to SEQ ID NO: 55.

69. The Fc-based chimeric protein complex of claim 59, wherein the Fc-based chimeric protein complex comprises a first amino acid sequence having at least 95%, or at least 98%, or at least 99% identity to SEQ ID NO: 56 and a second amino acid sequence having at least 95%, or at least 98%, or at least 99% identity to SEQ ID NO: 57.

70. The Fc-based chimeric protein complex of claim 59, wherein the Fc-based chimeric protein complex comprises a first amino acid sequence having at least 95%, or at least 98%, or at least 99% identity to SEQ ID NO: 58 and a second amino acid sequence having at least 95%, or at least 98%, or at least 99% identity to SEQ ID NO: 59.

71. The Fc-based chimeric protein complex of claim 59, wherein the Fc-based chimeric protein complex comprises a first amino acid sequence having at least 95%, or at least 98%, or at least 99% identity to any one of sequences selected from SEQ ID NOs: 60-65 and a second amino acid sequence having at least 95%, or at least 98%, or at least 99% identity to SEQ ID NO: 66.

72. The Fc-based chimeric protein complex of any one of claims 1-71, wherein the FLT3L domain is a single chain dimer of the formula A-B-C, wherein:
A is an amino acid sequence having at least 90% identity, or having at least 95% identity, or having at least 97% identity, or having at least 98% identity, or having at least 99% identity with any one of SEQ ID NOs: 2-5,
B is a flexible linker which is substantially comprised of glycine and serine residues, optionally wherein the flexible linker comprises (Gly₄Ser)ₙ, where n is from about 1 to about 8, optionally wherein the flexible linker comprises one or more of SEQ ID NO: 10-SEQ ID NO: 17, and
C is an amino acid sequence having at least 90% identity, or having at least 95% identity, or having at least 97% identity, or having at least 98% identity, or having at least 99% identity with any one of SEQ ID NOs: 2-5.

73. The Fc-based chimeric protein complex of claim 72, wherein the FLT3L domain comprises a L27D mutation.

74. A method for treating or preventing a cancer, comprising administering an effective amount of the Fc-based chimeric protein complex of any one of the above claims to a patient in need thereof.

75. The method of claim 74, wherein the cancer is selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

76. The method of claim 75, wherein the cancer is acute myeloid leukemia (AML).

77. A method for treating or preventing an autoimmune and/or neurodegenerative disease, comprising administering an effective amount of the Fc-based chimeric protein complex of any one of claims 1-73 to a patient in need thereof.

78. The method of claim 77, wherein the autoimmune and/or neurodegenerative disease is selected from multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, myasthenia gravis, Reiter's syndrome, and Grave's disease.

79. A recombinant nucleic acid composition encoding one or more Fc-based chimeric protein complexes of any one of claims 1-73, or a polypeptide constituent thereof.

80. A host cell comprising a nucleic acid of claim 79.

81. A chimeric protein comprising:
(i) one or more targeting moieties comprising a FMS-like tyrosine kinase 3 ligand (FLT3L) domain, wherein the FLT3L domain is a single chain dimer and;
(ii) one or more flexible linkers connecting elements (i) and (iii); and
(iii) a signaling agent or a modified form thereof.

82. The chimeric protein of claim 81, wherein the targeting moiety comprises the extracellular domain of FLT3L, or a portion thereof.

83. The chimeric protein of claim 81 or 82, wherein the targeting moiety comprises an amino acid sequence having at least 90% identity with any one of SEQ ID NOs: 2-5.

84. The chimeric protein of claim 83, wherein the targeting moiety comprises an amino acid sequence having at least 95% identity with any one of SEQ ID NOs: 2-5.

85. The chimeric protein of any one of claims 81-84, wherein the signaling agent is wild type human IFNα2, IFNα1, IFNβ, or IL-1β.

86. The chimeric protein of claim 85, wherein the signaling agent comprises an amino acid sequence having at least 95% identity with any one of SEQ ID NO: 6, 7, 38, 39, or 74.

87. The chimeric protein of claim 86, wherein the signaling agent comprises an amino acid sequence of any one of SEQ ID NO: 6, 7, 38, 39, or 74.

88. The chimeric protein of any one of claims 81-87, wherein the signaling agent is modified to comprise one or more mutations.

89. The chimeric protein of claim 88, wherein the one or more mutations confer improved safety as compared to a wild type signaling agent, or reduced affinity for the signaling agent's receptor, or reduced bioactivity for the signaling agent's receptor, or allow for attenuation of the signaling agent's activity.

90. The chimeric protein of claim 88, wherein the one or more mutations confer reduced affinity or activity that is restorable by attachment to one or more targeting moiety.

91. The chimeric protein of any one of claims 81-90, wherein:
(a) the signaling agent is a mutant human IFNα2 comprising an amino acid sequence having at least 95% identity with SEQ ID NO: 6 or 7 and wherein the mutant human IFNα2 has one or more mutations that confer improved safety as compared to a wild type IFNα2 having an amino acid sequence of SEQ ID NO: 6 or 7, optionally wherein the human IFNα2 has one or more mutations at positions 144 to 154 with respect to SEQ ID NO: 6 or 7, optionally wherein the human IFNα2 has: one or more mutations at positions L15, A19, R22, R23, L26, F27, L30, L30, K31, D32, R33, H34, D35, Q40, H57, E58, Q61, F64, N65, T69, L80, Y85, Y89, D114, L117, R120, R125, K133, K134, R144, A145, M148, R149, S152, L153, and N156 with respect to SEQ ID NO: 6 or 7, optionally wherein the mutant human IFNα2 has one or more mutations at position R33, T106, R144, A145, M148, R149, and L153 with respect to SEQ ID NO: 6 or 7, optionally wherein the mutation is one or more of L15A, A19W, R22A, R23A, L26A, F27A, L30A, L30V, K31A, D32A, R33K, R33A, R33Q, H34A, D35A, Q40A, H57Y, E58N, Q61S, F64A, N65A, T69A, L80A, Y85A, Y89A, D114R, L117A, R120A, R125A, K133A, K134A, R144A, A145G, A145M, M148A, R149A, S152A, L153A, and N156A with respect to SEQ ID NO: 6 or 7, optionally wherein the mutant human IFNα2 has one or more mutations selected from R33A, T106X₃, R120E, R144X₁ A145X₂, M148A, R149A, and L153A with respect to amino acid sequence of SEQ ID NO: 6 or 7, wherein X₁ is selected from A, S, T, Y, L, and I, wherein X₂ is selected from G, H, Y, K, and D, and wherein X₃ is selected from A and E;
(b) the signaling agent is a mutant human IFNβ comprising an amino acid sequence having at least 95% identity with SEQ ID NO: 38 and wherein the mutant human IFNβ has one or more mutations that confer improved safety as compared to a wild type IFNβ having an amino acid sequence of SEQ ID NO: 38, optionally wherein the mutation is one or more of W22G, R27G, L32A, L32G, R35A, R35G, V148G, L151G, R152A, R152G with respect to amino acid sequence of SEQ ID NO: 38;
(c) the signaling agent is a mutant human IL-1β comprising an amino acid sequence having at least 95% identity with SEQ ID NO: 39 and wherein the mutant human IL-1β has one or more mutations that confer improved safety as compared to a wild type IL-1β having an amino acid sequence of SEQ ID NO: 39, optionally wherein the mutation is one or more of A117G/P118G, R120G, R120A, L122A, T125G/L126G, R127G, Q130A, Q130W, Q131G, K132A, S137G/Q138Y, L145G, H146A, H146G, H146E, H146N, H146R, L145A/L147A, Q148E, Q148G, Q148L, Q148G/Q150G, Q150G/D151A, M152G, F162A, F162A/Q164E, F166A, Q164E/E167K, N169G/D170G, 1172A, V174A, K208E, K209A, K209D, K209A/K210A, K219S, K219Q, E221S, E221K, E221S/N224A, N224S/K225S, E244K and N245Q with respect to amino acid sequence of SEQ ID NO: 39 or
(d) the signaling agent is wild type or a mutant human IFN-α1 which comprises an amino acid sequence having at least about 90%, or at least about 93%, or at least about 95%, or at least about 97%, or at least about 98%, or at least about 99%, or 100% identity with SEQ ID NO: 74, optionally wherein the IFN-α1 comprises:
(i) one or more mutations which confer reduced affinity for interferon-α/β receptor (IFNAR), optionally selected from a substitution at L15, A19, R23, S25, L30, D32, R33, H34, Q40, D115, L118, K121, R126, E133, K134, K135, R145, A146, M149, R150, S153, L154, and N157 or a combination thereof, wherein the positions are in reference to SEQ ID NO: 74, optionally selected from the group consisting of L15A, A19W, R23A, S25A, L30A, L30V, D32A, R33K, R33A, R33Q, H34A, Q40A, D115R, L118A, K121A, K121E, R126A, R126E, E133A, K134A, K135A, R145A, R145D, R145E, R145G, R145H, R145I, R145K, R145L, R145N, R145Q, R145S, R145T, R145V, R145Y, A146D, A146E, A146G, A146H, A146I, A146K, A146L, A146M, A146N, A146Q, A146R, A146S, A146T, A146V, A146Y, M149A, M149V, R150A, S153A, L154A, N157A, L30A-H58Y-E59N-Q62S, R33A-H58Y-E59N-Q625, M149A-H58Y-E59N-Q62S, L154A-H58Y-E59N-Q62S, R145A-H58Y-E59N-Q62S, D115A-R121A, L118A-R121A, L118A-R121A-K122A, R121A-K122A, and R121E-K122E and/or
(ii) one or more mutations which affect aggregation, optionally selected from a substitution or deletion of one or more of position C1, C29, C86, C99, C139 in reference to SEQ ID NO: 74, optionally selected from C86S, C86A, and C86Y.

92. The chimeric protein of any one of claims 81-91, wherein the flexible linker is substantially comprised of glycine and serine residues, optionally wherein the flexible linker comprises (Gly₄Ser)ₙ, where n is from about 1 to about 8, optionally wherein the flexible linker comprises one or more of SEQ ID NO: 10-SEQ ID NO: 17.

93. The chimeric protein of any one of claims 81-92, wherein the targeting moiety comprises the extracellular domain of FLT3L, or a portion thereof and a mutation that reduces intermolecular extracellular domain dimerization, optionally being a substitution at position L27 of any one of SEQ ID NOs: 2-4, optionally being L27D.

94. The chimeric protein of any one of claims 81-93, further comprising an additional targeting moiety and/or additional signaling agent or a modified form thereof.

95. A recombinant nucleic acid composition encoding one or more chimeric proteins of any one of claims 81-94.

96. A host cell comprising the recombinant nucleic acid of claim 95.

97. The chimeric protein of any one of claims 81-94, wherein the chimeric protein is suitable for use in a patient having one or more of: cancer, infections, immune disorders, autoimmune and/or neurodegenerative disease, cardiovascular diseases, wound, ischemia-related diseases, and/or metabolic diseases.

98. A method for treating or preventing cancer, infections, immune disorders, autoimmune and/or neurodegenerative disease, cardiovascular diseases, wound, ischemia-related diseases, and/or metabolic diseases comprising administering an effective amount of the chimeric protein of any of claims 81-94 to a patient in need thereof.

99. An Fc-based chimeric protein complex comprising:
(i) one or more targeting moieties comprising a FMS-like tyrosine kinase 3 ligand (FLT3L) domain, wherein the FLT3L domain is a single chain dimer and
(ii) an Fc domain, the Fc domain optionally having one or more mutations that reduces or eliminates one or more effector functions of the Fc domain, promotes Fc chain pairing in the Fc domain, and/or stabilizes a hinge region in the Fc domain.

100. The Fc-based chimeric protein complex of claim 99, wherein the single chain dimeric FLT3L is attached to one Fc chain of the Fc domain.

101. The Fc-based chimeric protein complex of any one of claims 99-100, wherein the single chain dimeric FLT3L comprises the extracellular domain of FLT3L, or a portion thereof.

102. The Fc-based chimeric protein complex of any one of claims 99-101, wherein the single chain dimeric FLT3L comprises an amino acid sequence having at least 90% identity with any one of SEQ ID NOs: 2-5, or at least 95% identity with any one of SEQ ID NOs: 2-5, or at least 98% identity with any one of SEQ ID NOs: 2-5.

103. The Fc-based chimeric protein complex of any one of claims 98-102, wherein the targeting moiety comprises the extracellular domain of FLT3L, or a portion thereof and a mutation that reduces intermolecular extracellular domain dimerization, optionally being a substitution at position L27 of any one of SEQ ID NOs: 2-4, optionally being L27D.

104. The Fc-based chimeric protein complex of any one of claims 98-103, further comprising an additional targeting moiety.

105. A recombinant nucleic acid composition encoding one or more chimeric proteins of any one of claims 99-104.

106. A host cell comprising the recombinant nucleic acid of claim 105.

107. The Fc-based chimeric protein complex of any one of claims 99-104, wherein the chimeric protein is suitable for use in a patient having one or more of: cancer, infections, immune disorders, autoimmune and/or neurodegenerative disease, cardiovascular diseases, wound, ischemia-related diseases, and/or metabolic diseases.

108. A method for treating or preventing cancer, infections, immune disorders, autoimmune and/or neurodegenerative disease, cardiovascular diseases, wound, ischemia-related diseases, and/or metabolic diseases comprising administering an effective amount of the Fc-based chimeric protein complex of any of claims 99-104 to a patient in need thereof.


SEQUENCE LISTING (74 sequences)

SEQ ID NO: 1 - protein, 235 aa, Artificial Sequence
  Synthetic Sequence
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 2 - protein, 158 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQP

SEQ ID NO: 3 - protein, 155 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTA

SEQ ID NO: 4 - protein, 134 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQP

SEQ ID NO: 5 - protein, 129 aa, Artificial Sequence
  Synthetic Sequence
CSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQC

SEQ ID NO: 6 - protein, 165 aa, Artificial Sequence
  Synthetic Sequence
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 7 - protein, 164 aa, Artificial Sequence
  Synthetic Sequence
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEWRAEIMRSFSLSTNLQESLRSKE

SEQ ID NO: 8-9 - skipped (2 entries)

SEQ ID NO: 10 - protein, 5 aa, Artificial Sequence
  Synthetic Sequence
GGGGS

SEQ ID NO: 11 - protein, 10 aa, Artificial Sequence
  Synthetic Sequence
GGGGSGGGGS

SEQ ID NO: 12 - protein, 15 aa, Artificial Sequence
  Synthetic Sequence
GGGGSGGGGSGGGGS

SEQ ID NO: 13 - protein, 20 aa, Artificial Sequence
  Synthetic Sequence
GGGGSGGGGSGGGGSGGGGS

SEQ ID NO: 14 - protein, 25 aa, Artificial Sequence
  Synthetic Sequence
GGGGSGGGGSGGGGSGGGGSGGGGS

SEQ ID NO: 15 - protein, 30 aa, Artificial Sequence
  Synthetic Sequence
GGGGSGGGGSGGGGSGGGGSGGGGSGGGGS

SEQ ID NO: 16 - protein, 35 aa, Artificial Sequence
  Synthetic Sequence
GGGGSGGGGSGGGGSGGGGSGGGGSGGGGSGGGGS

SEQ ID NO: 17 - protein, 40 aa, Artificial Sequence
  Synthetic Sequence
GGGGSGGGGSGGGGSGGGGSGGGGSGGGGSGGGGSGGGGS

SEQ ID NO: 18 - protein, 16 aa, Artificial Sequence
  Synthetic Sequence
GGSGGSGGGGSGGGGS

SEQ ID NO: 19 - protein, 8 aa, Artificial Sequence
  Synthetic Sequence
GGGGGGGG

SEQ ID NO: 20 - protein, 6 aa, Artificial Sequence
  Synthetic Sequence
GGGGGG

SEQ ID NO: 21 - protein, 5 aa, Artificial Sequence
  Synthetic Sequence
EAAAK

SEQ ID NO: 22 - protein, 10 aa, Artificial Sequence
  Synthetic Sequence
EAAAKEAAAK

SEQ ID NO: 23 - protein, 15 aa, Artificial Sequence
  Synthetic Sequence
EAAAKEAAAKEAAAK

SEQ ID NO: 24 - protein, 12 aa, Artificial Sequence
  Synthetic Sequence
AEAAAKEAAAKA

SEQ ID NO: 25 - protein, 17 aa, Artificial Sequence
  Synthetic Sequence
AEAAAKEAAAKEAAAKA

SEQ ID NO: 26 - protein, 22 aa, Artificial Sequence
  Synthetic Sequence
AEAAAKEAAAKEAAAKEAAAKA

SEQ ID NO: 27 - protein, 27 aa, Artificial Sequence
  Synthetic Sequence
AEAAAKEAAAKEAAAKEAAAKEAAAKA

SEQ ID NO: 28 - protein, 46 aa, Artificial Sequence
  Synthetic Sequence
AEAAAKEAAAKEAAAKEAAAKALEAEAAAKEAAAKEAAAKEAAAKA

SEQ ID NO: 29 - protein, 5 aa, Artificial Sequence
  Synthetic Sequence
PAPAP

SEQ ID NO: 30 - protein, 18 aa, Artificial Sequence
  Synthetic Sequence
KESGSVSSEQLAQFRSLD

SEQ ID NO: 31 - protein, 14 aa, Artificial Sequence
  Synthetic Sequence
EGKSSGSGSESKST

SEQ ID NO: 32 - protein, 12 aa, Artificial Sequence
  Synthetic Sequence
GSAGSAAGSGEF

SEQ ID NO: 33 - protein, 5 aa, Artificial Sequence
  Synthetic Sequence
GGGSE

SEQ ID NO: 34 - protein, 5 aa, Artificial Sequence
  Synthetic Sequence
GSESG

SEQ ID NO: 35 - protein, 5 aa, Artificial Sequence
  Synthetic Sequence
GSEGS

SEQ ID NO: 36 - protein, 35 aa, Artificial Sequence
  Synthetic Sequence
GEGGSGEGSSGEGSSSEGGGSEGGGSEGGGSEGGS

SEQ ID NO: 37 - protein, 4 aa, Artificial Sequence
  Synthetic Sequence
CPPC

SEQ ID NO: 38 - protein, 166 aa, Artificial Sequence
  Synthetic Sequence
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 39 - protein, 153 aa, Artificial Sequence
  Synthetic Sequence
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 40 - protein, 446 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPSGGSGGSGGSGGSGGSGGSGGSGGSGGSGGSGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 41 - protein, 452 aa, Artificial Sequence
  Synthetic Sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 42 - protein, 4 aa, Artificial Sequence
  Synthetic Sequence
CPPC

SEQ ID NO: 43 - protein, 9 aa, Artificial Sequence
  Synthetic Sequence
DKTHTCPPC

SEQ ID NO: 44 - protein, 14 aa, Artificial Sequence
  Synthetic Sequence
EPKSCDKTHTCPPC

SEQ ID NO: 45 - protein, 14 aa, Artificial Sequence
  Synthetic Sequence
EPKSSDKTHTCPPC

SEQ ID NO: 46 - protein, 401 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 47 - protein, 626 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 48 - protein, 386 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 49 - protein, 611 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 50 - protein, 376 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 51 - protein, 601 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 52 - protein, 401 aa, Artificial Sequence
  Synthetic Sequence
TQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 53 - protein, 626 aa, Artificial Sequence
  Synthetic Sequence
TQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 54 - protein, 386 aa, Artificial Sequence
  Synthetic Sequence
TQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 55 - protein, 611 aa, Artificial Sequence
  Synthetic Sequence
TQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 56 - protein, 376 aa, Artificial Sequence
  Synthetic Sequence
TQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 57 - protein, 601 aa, Artificial Sequence
  Synthetic Sequence
TQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 58 - protein, 579 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEASGGGGSGGGGSGGGGSTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 59 - protein, 452 aa, Artificial Sequence; identical to SEQ ID NO: 41
  Synthetic Sequence

SEQ ID NO: 60 - protein, 584 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPGGGGSGGGGSGGGGSTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 61 - protein, 584 aa, Artificial Sequence
  Synthetic Sequence
TQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPGGGGSGGGGSGGGGSTQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 62 - protein, 574 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATGGGGSGGGGSGGGGSTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 63 - protein, 574 aa, Artificial Sequence
  Synthetic Sequence
TQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATGGGGSGGGGSGGGGSTQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - protein, 540 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPGGGGSGGGGSGGGGSTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 65 - protein, 540 aa, Artificial Sequence
  Synthetic Sequence
TQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPGGGGSGGGGSGGGGSTQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPGGSGGSGGSGGSGGSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 66 - protein, 452 aa, Artificial Sequence; identical to SEQ ID NO: 41
  Synthetic Sequence

SEQ ID NO: 67 - protein, 522 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPGGGGSGGGGSGGGGSTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 68 - protein, 522 aa, Artificial Sequence
  Synthetic Sequence
TQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPGGGGSGGGGSGGGGSTQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 69 - protein, 512 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATGGGGSGGGGSGGGGSTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 70 - protein, 512 aa, Artificial Sequence
  Synthetic Sequence
TQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATGGGGSGGGGSGGGGSTQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 71 - protein, 478 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPGGGGSGGGGSGGGGSTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 72 - protein, 478 aa, Artificial Sequence
  Synthetic Sequence
TQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPGGGGSGGGGSGGGGSTQDCSFQHSPISSDFAVKIRELSDYLDQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPGGSGGSGGSGGSGGSGGSGGSGGSGGSGGSCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVEETPLMKEDSILAVRKYFQRITLYLKEKKYSPCAWEVVRAEIMASFSLSTNLQESLRSKE

SEQ ID NO: 73 - protein, 385 aa, Artificial Sequence
  Synthetic Sequence
TQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAVDGGSGGSGGSGGSGGSGGSRSGGSGGSGGSGGSGGSGGSGGSGGSGGSGGSGGSGGSGGSAAAMCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 74 - protein, 166 aa, Artificial Sequence
  Synthetic Sequence
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE